(19) United States
(12) Patent Application Publication
(10) Pub. No.: US 2024/0040925 A1
(43) Pub. Date: Feb. 1, 2024

(54) METAL COMPLEX AND USE THEREOF

(71) Applicant: GUANGDONG AGLAIA OPTOELECTRONIC MATERIALS CO., LTD, Foshan, Guangdong (CN)

(72) Inventors: Liangliang YAN, Foshan (CN); Lei DAI, Foshan (CN); Lifei CAI, Foshan (CN)

(73) Assignee: GUANGDONG AGLAIA OPTOELECTRONIC MATERIALS CO., LTD, Foshan, Guangdong (CN)

(21) Appl. No.: 18/038,672

(22) PCT Filed: Oct. 24, 2021
(86) PCT No.: PCT/CN2021/125928

§ 371 Date: May 24, 2023

(30) Foreign Application Priority Data
Dec. 4, 2020 (CN) 202011397748.1
Sep. 26, 2021 (CN) 202111129537.4

(51) Int. Cl. H10K 85/30; C07F 15/00; C09K 11/06

(52) U.S. Cl. CPC H10K 85/342; C07F 15/0033; C09K 11/06; H10K 50/12

(57) ABSTRACT

The present invention relates to a metal complex and application thereof. The metal complex has a general formula of Ir(La)(Lb)(Lc) and a structure as shown in a formula (1). The metal complex has the advantages of low sublimation temperature, good optical and electrical stability, high luminous efficiency, long service life, and high color saturation, and can be used in organic light-emitting devices. The metal complex can particularly serve as a red light-emitting phosphorescent material, and has the potential of being applied in the AMOLED industry.

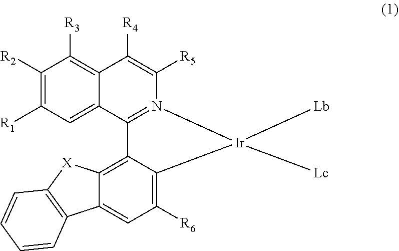

### TECHNICAL FIELD

The present invention relates to the technical field of organic electroluminescence, in particular to an organic luminescent material, and specially relates to a metal complex and application thereof in an organic electroluminescent device.

### BACKGROUND

At present, as a new-generation display technology, an organic electroluminescent device (OLED) has attracted more and more attention in display and lighting technologies, thus having a wide application prospect. However, compared with market application requirements, properties, such as luminous efficiency, driving voltage, and service life of OLED devices still need to be strengthened and improved.

In generally, the OLED devices include various organic functional material films with different functions sandwiched between metal electrodes as basic structures, which are similar to sandwich structures. Under the driving of a current, holes and electrons are injected from a cathode and an anode, respectively. After moving a certain distance, the holes and the electrons are compounded in a light-emitting layer, and then released in the form of light or heat to achieve luminescence of the OLED.

However, organic functional materials are core components of the OLED devices, and the thermal stability, photochemical stability, electrochemical stability, quantum yield, film forming stability, crystallinity, and color saturation of the materials are main factors affecting properties of the devices.

In general, the organic functional materials include fluorescent materials and phosphorescent materials. The fluorescent materials are usually organic small-molecule materials, which can only use 25% of singlet luminescence, thus having low luminous efficiency. Meanwhile, due to a spin-orbit coupling effect caused by a heavy atom effect, the phosphorescent materials can use 25% of singlet excitons, and can also use 75% of energy of triplet excitons, so that the luminous efficiency can be greatly improved. However, compared with the fluorescent materials, the phosphorescent materials are developed later, and the thermal stability, service life, and color saturation of the materials need to be improved. Thus, the phosphorescent materials are a challenging topic. Various organic metal compounds have been developed to serve as the phosphorescent materials. For example, according to an invention patent document CN107973823, a quinoline iridium compound is disclosed. However, the color saturation and device properties, especially luminous efficiency and device service life, of the compound need to be improved. According to an invention patent document CN106459114, an iridium compound coordinated with a β-dione coordination group is disclosed. However, the compound has high sublimation temperature and low color saturation. In particular, the device performance is unsatisfactory, which needs to be further improved. According to an invention patent CN109721628, a compound with a fluorenyl thiophenpyrimidine structure and an organic electroluminescent device and compound including the compound are disclosed. According to invention patents CN111377969A and CN111620910A, a complex with a dibenzofuran-isoquinoline structure and an organic electroluminescent device and compound including the complex are disclosed.

However, a novel material capable of improving properties of organic electroluminescent devices is still expected to be further developed.

### SUMMARY

In order to solve the above problems, purposes of the present invention are to provide an organic electroluminescent device having high properties and to provide a novel material capable of realizing the organic electroluminescent device.

In order to achieve the above purposes, the inventor has conducted in-depth studies repeatedly and found that an organic electroluminescent device having high properties can be obtained by using a metal complex having a structure as shown in the following formula (1) as a ligand.

One of the purposes of the present invention is to provide a metal complex. The metal complex has the advantages of low sublimation temperature, high optical and electrochemical stability, high color saturation, high luminous efficiency, and long device service life, and can be used in organic electroluminescent devices. In particular, the metal complex has the potential for application in the OLED industry as a red light-emitting dopant.

A metal complex has a general formula of Ir(La)(Lb)(Lc) and a structural formula as shown in a formula (1):

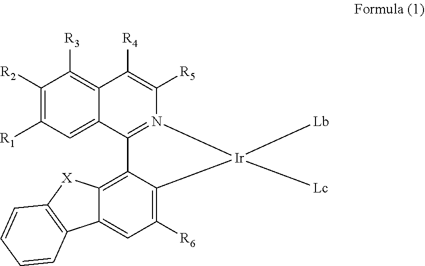

where

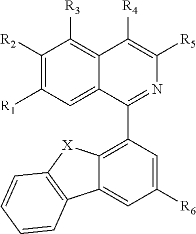

is a ligand La;
X is independently selected from O, S, and Se;
R₁-R₅ are independently selected from hydrogen, deuterium, halogen, substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, substituted or unsubstituted heterocycloalkyl containing 3-20 ring forming carbon atoms, substituted or unsubstituted C₃-C₃₀ alkylsilyl, substituted or unsubstituted C₁-C₁₀ alkoxyl, substituted or unsubstituted C₇-C₃₀ aralkyl, substituted or unsubstituted C₆-C₃₀ aryloxyl, substituted or unsubstituted C₂-C₂₀ alkenyl, substituted or unsubstituted C₂-C₂₀ alkynyl, substituted or unsubstituted C₆-C₃₀ aryl, substituted or unsubstituted C₃-C₃₀ heteroaryl, substituted or unsubstituted C₃-C₃₀ arylsilyl, substituted or unsubstituted C₀-C₂₀ alkylamino, cyano, nitrile, isonitrile, and phosphino;
at least one of the R₁-R₅ is F, and one of the R₁-R₅ is substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, or substituted or unsubstituted heterocycloalkyl containing 3-20 ring forming carbon atoms;
R₆ is substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, or substituted or unsubstituted heterocycloalkyl containing 3-20 ring forming carbon atoms;
the “substituted” refers to substitution with deuterium, F, Cl, Br, C₁-C₄ alkyl, C₁-C₄ alkoxyl, C₃-C₆ cycloalkyl, amino substituted with C₁-C₄ alkyl, cyano, nitrile, isonitrile, or phosphino;
the heteroalkyl, the heterocycloalkyl, or the heteroaryl includes at least one of S, O, and N heteroatoms;
Lb and Lc are both a monoanionic bidentate ligand, any two of the La, the Lb, and the Lc are connected to each other to form a multidentate ligand, or the La, the Lb, and the Lc are connected by a group;
and at least two of the La, the Lb, and the Lc are the same.

As a preferred metal complex, the Lb has a structure as shown in a formula (2):

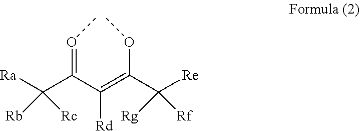

where a dotted line refers to a position connected to metal Ir;
Rₐ-R_{g} are independently selected from hydrogen, deuterium, halogen, substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, and substituted or unsubstituted heterocycloalkyl containing 3-20 ring forming carbon atoms, or any two of Rₐ, R_{b}, and R_{c} are connected to each other to form an aliphatic ring structure, and any two of Rₑ, R_{f}, and R_{g} are connected to each other to form an aliphatic ring structure; and the “substituted” refers to substitution with deuterium, F, Cl, Br, C₁-C₄ alkyl, C₁-C₄ alkoxyl, C₃-C₆ cycloalkyl, amino substituted with C₁-C₄ alkyl, cyano, nitrile, isonitrile, or phosphino.

As a preferred metal complex, the Lc and the La have the same structure, so that a (La)₂Ir(Lb) structure is formed.

The Rₐ, the R_{b}, and the R_{c} are the same as the Rₑ, the R_{f}, and the R_{g}, respectively.

The Rₐ, the R_{b}, the R_{c}, the Rₑ, the R_{f}, and the R_{g} are independently selected from hydrogen, deuterium, halogen, substituted or unsubstituted alkyl containing 1-10 carbon atoms on a main chain, and substituted or unsubstituted cycloalkyl containing 3-20 ring forming carbon atoms, or any two of the Rₐ, the R_{b}, and the R_{c} are connected to each other to form an aliphatic ring structure, and any two of the Rₑ, the R_{f}, and the R_{g} are connected to each other to form an aliphatic ring structure; the “substituted” refers to substitution with deuterium, F, Cl, Br, C₁-C₄ alkyl, or C₃-C₆ cycloalkyl; and R_{d} is selected from hydrogen, deuterium, halogen, and substituted or unsubstituted alkyl containing 1-10 carbon atoms on a main chain.

As a preferred metal complex, the R₆ is substituted or unsubstituted alkyl containing no more than 4 carbon atoms on a main chain, or substituted or unsubstituted cycloalkyl containing no more than 6 ring forming carbon atoms.

As a preferred metal complex, the F is not positioned at the R₅.

The X is an O atom.

As a preferred metal complex, one of the R₁-R₅ is F, another one is substituted or unsubstituted alkyl containing no more than 4 carbon atoms on a main chain, or substituted or unsubstituted cycloalkyl containing no more than 6 ring forming carbon atoms, and the other three are hydrogen.

As a preferred metal complex, when one of the R₁-R₅ is F, another one is branched alkyl substituted with C₁-C₄ alkyl containing no more than 4 carbon atoms on a main chain.

As a preferred metal complex, the La is independently selected from one of the following structural formulas, corresponding parts or complete deuterides thereof, or corresponding parts or complete fluorides thereof:

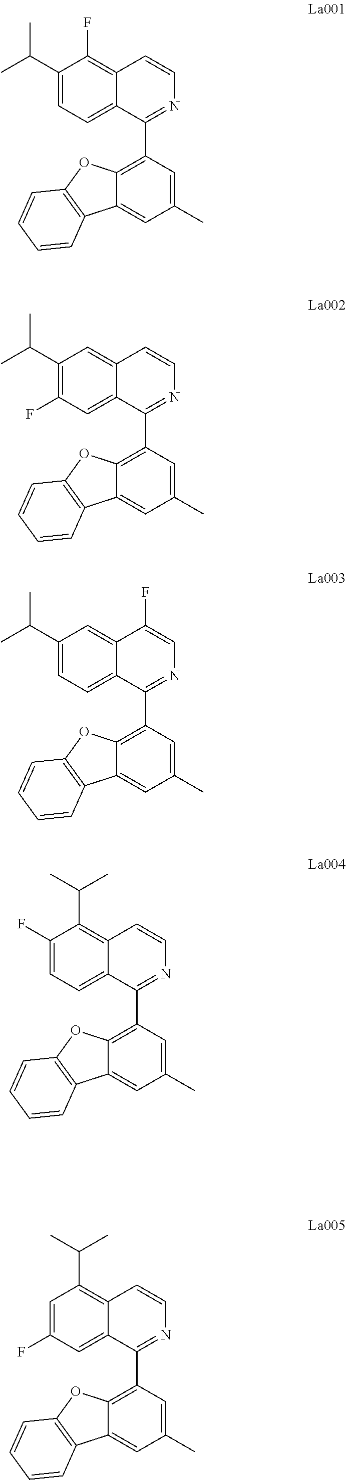

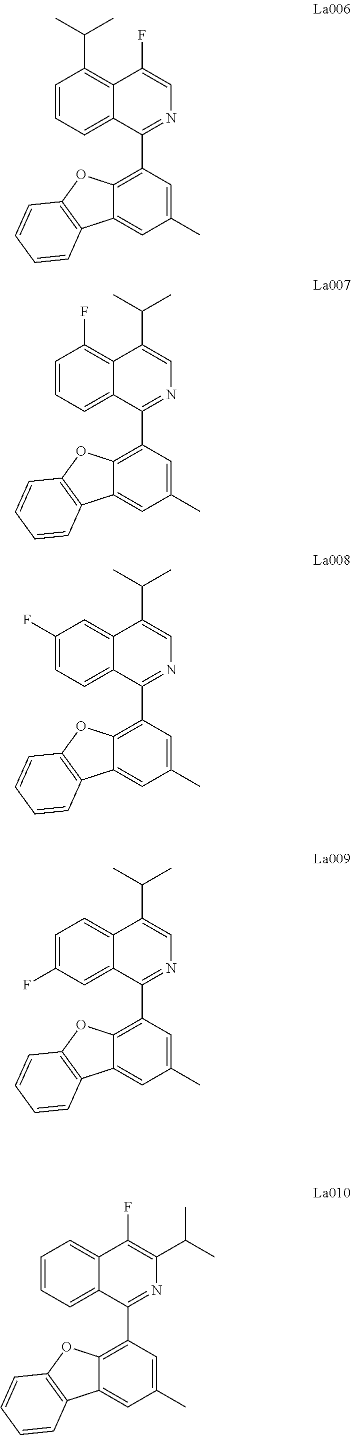 [SMILES: Cc1cc(-c2ncc(F)c3c(C(C)C)cccc23)c2oc3ccccc3c2c1 ; Cc1cc(-c2ncc(C(C)C)c3c(F)cccc23)c2oc3ccccc3c2c1 ; Cc1cc(-c2ncc(C(C)C)c3cc(F)ccc23)c2oc3ccccc3c2c1 ; Cc1cc(-c2ncc(C(C)C)c3ccc(F)cc23)c2oc3ccccc3c2c1 ; Cc1cc(-c2nc(C(C)C)c(F)c3ccccc23)c2oc3ccccc3c2c1]

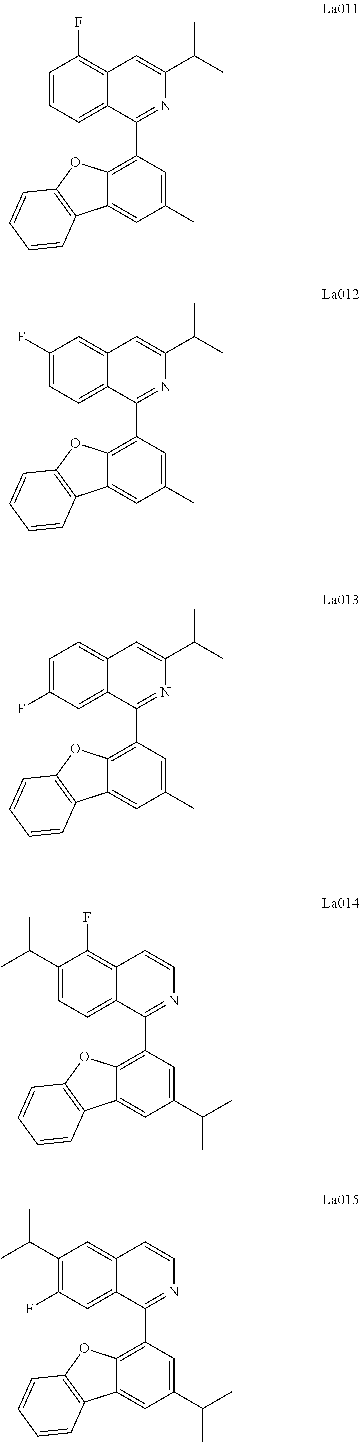

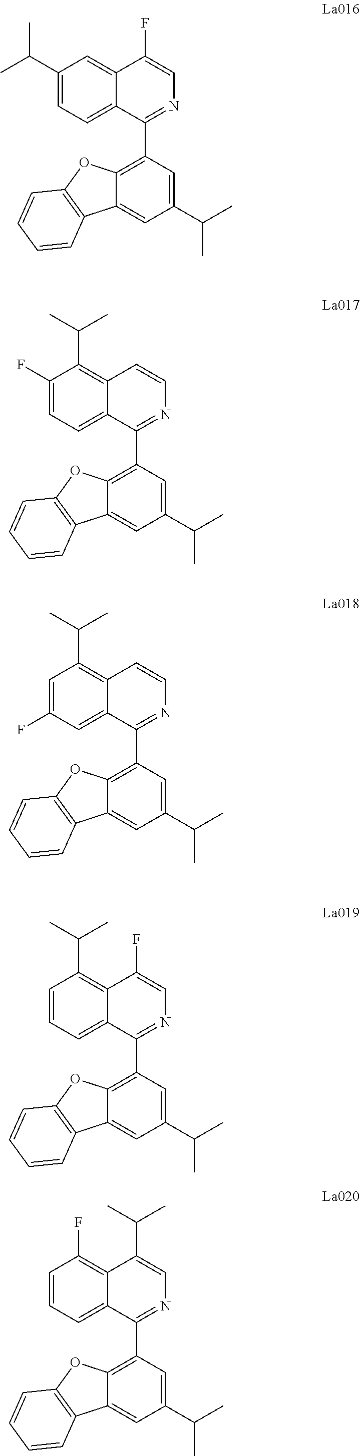

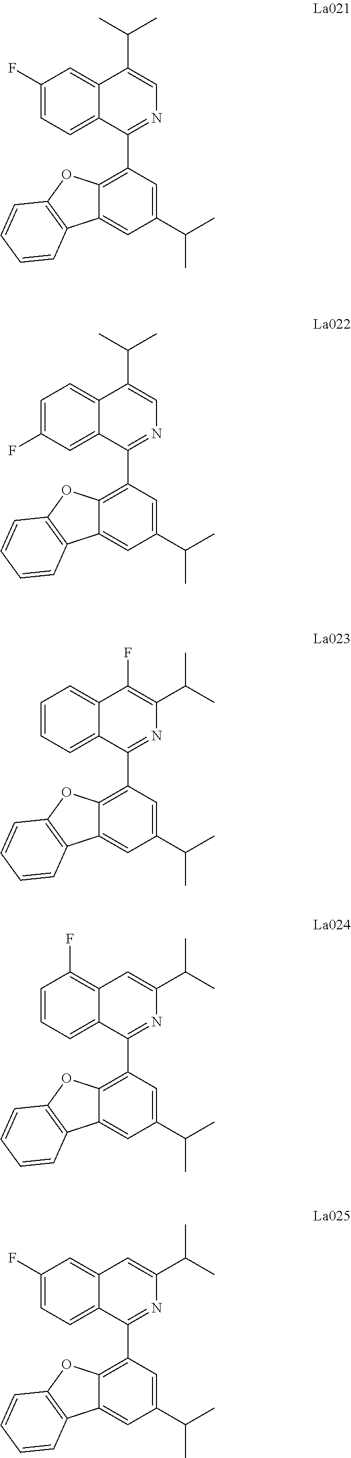 [SMILES: CC(C)c1cc(-c2ncc(C(C)C)c3cc(F)ccc23)c2oc3ccccc3c2c1 ; CC(C)c1cc(-c2ncc(C(C)C)c3ccc(F)cc23)c2oc3ccccc3c2c1 ; CC(C)c1cc(-c2nc(C(C)C)c(F)c3ccccc23)c2oc3ccccc3c2c1 ; CC(C)c1cc(-c2nc(C(C)C)cc3c(F)cccc23)c2oc3ccccc3c2c1 ; CC(C)c1cc(-c2nc(C(C)C)cc3cc(F)ccc23)c2oc3ccccc3c2c1]

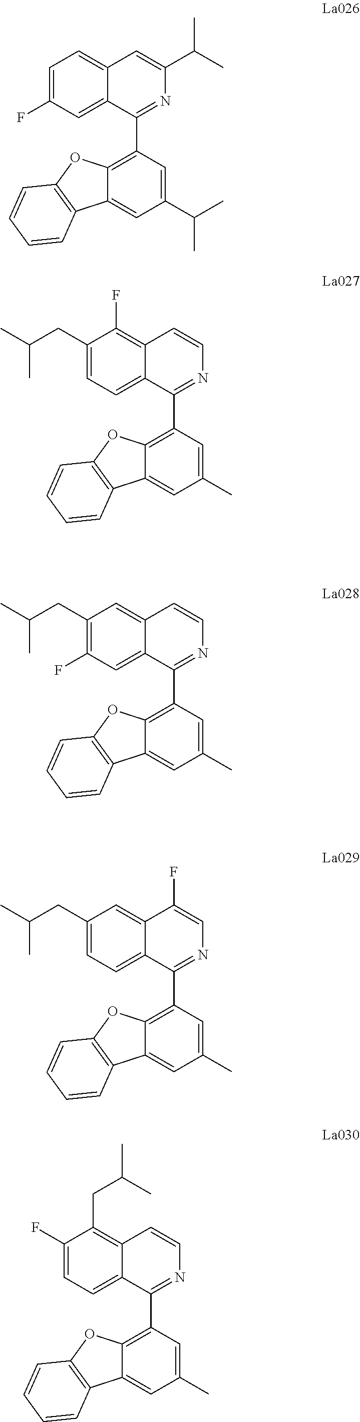

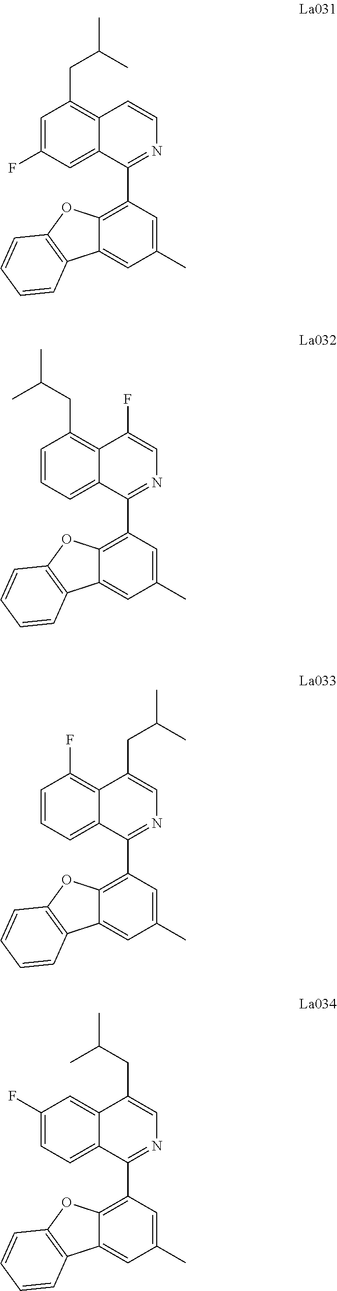 [SMILES: Cc1cc(-c2nccc3c(CC(C)C)cc(F)cc23)c2oc3ccccc3c2c1 ; Cc1cc(-c2ncc(F)c3c(CC(C)C)cccc23)c2oc3ccccc3c2c1 ; Cc1cc(-c2ncc(CC(C)C)c3c(F)cccc23)c2oc3ccccc3c2c1 ; Cc1cc(-c2ncc(CC(C)C)c3cc(F)ccc23)c2oc3ccccc3c2c1]

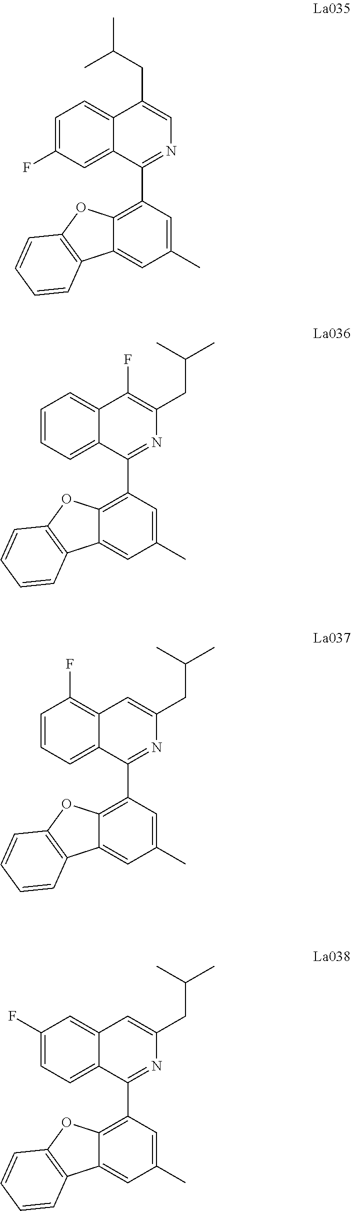 [SMILES: Cc1cc(-c2ncc(CC(C)C)c3ccc(F)cc23)c2oc3ccccc3c2c1 ; Cc1cc(-c2nc(CC(C)C)c(F)c3ccccc23)c2oc3ccccc3c2c1 ; Cc1cc(-c2nc(CC(C)C)cc3c(F)cccc23)c2oc3ccccc3c2c1 ; Cc1cc(-c2nc(CC(C)C)cc3cc(F)ccc23)c2oc3ccccc3c2c1]

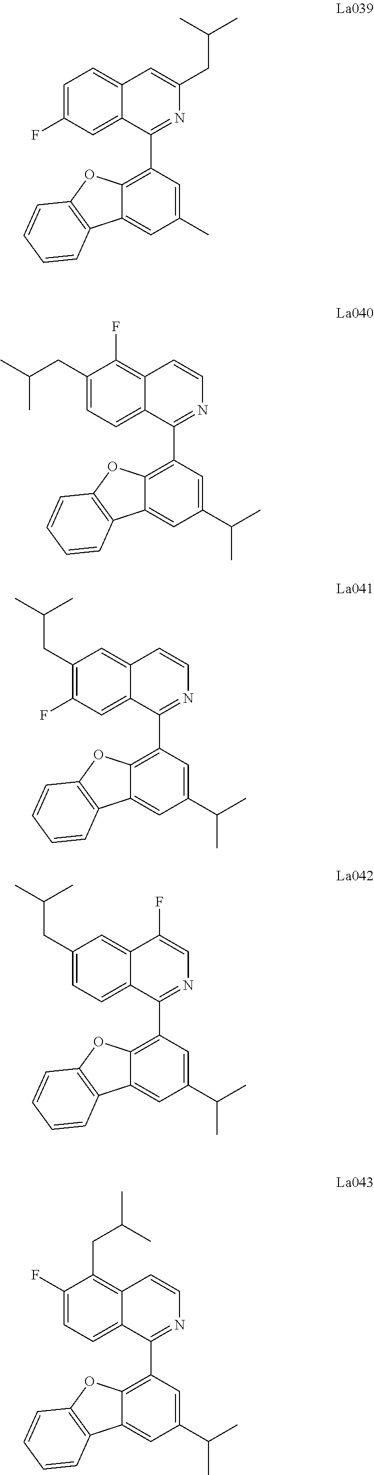

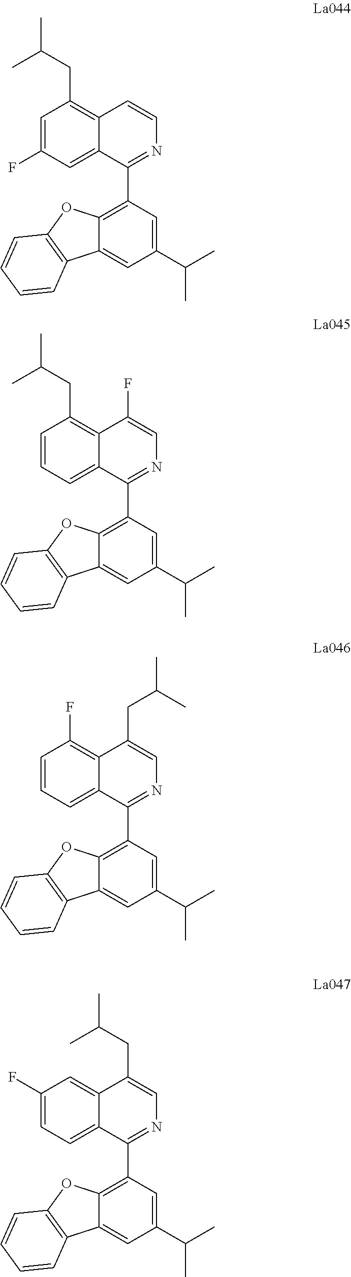

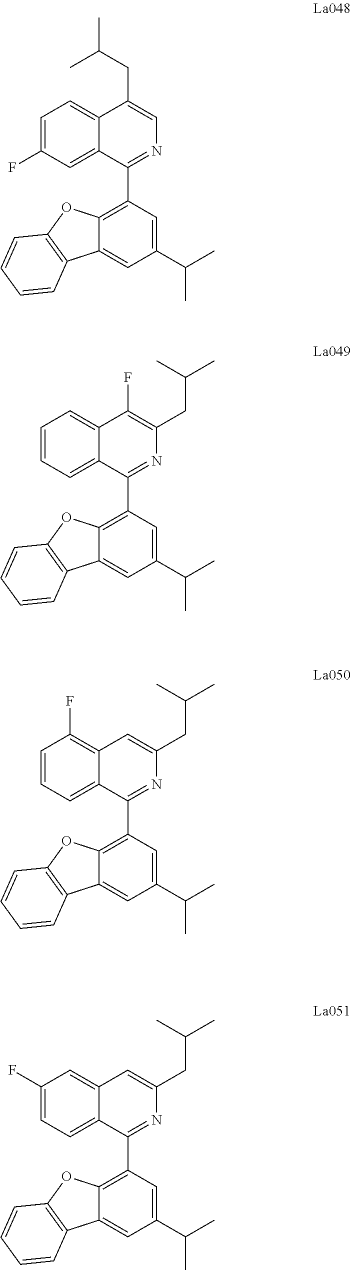 [SMILES: CC(C)Cc1cnc(-c2cc(C(C)C)cc3c2oc2ccccc23)c2cc(F)ccc12 ; CC(C)Cc1nc(-c2cc(C(C)C)cc3c2oc2ccccc23)c2ccccc2c1F ; CC(C)Cc1cc2c(F)cccc2c(-c2cc(C(C)C)cc3c2oc2ccccc23)n1 ; CC(C)Cc1cc2cc(F)ccc2c(-c2cc(C(C)C)cc3c2oc2ccccc23)n1]

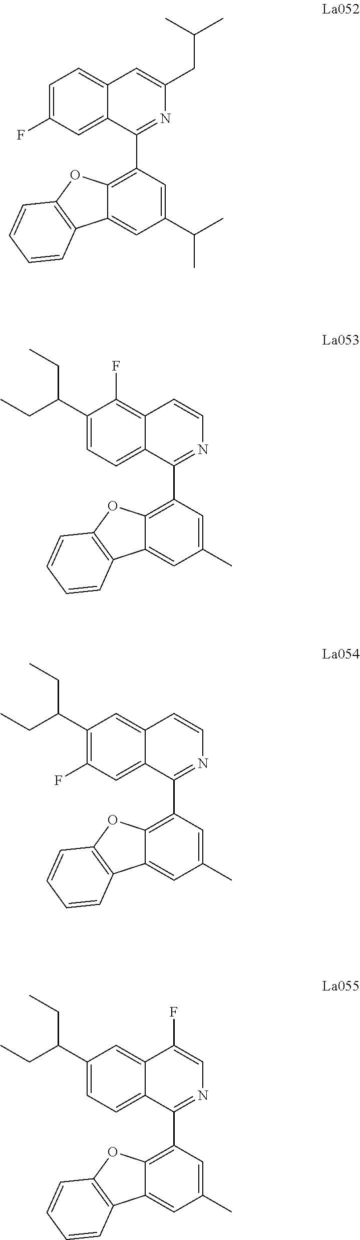

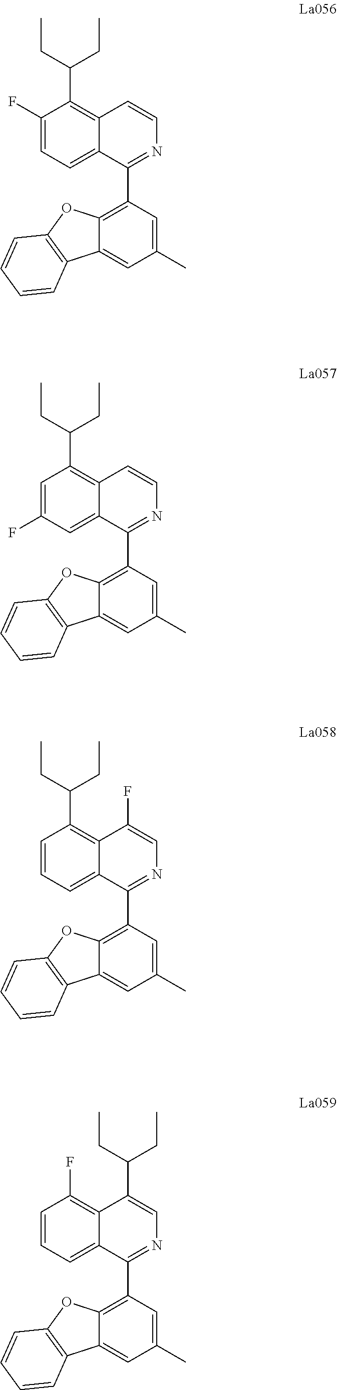 [SMILES: CCC(CC)c1c(F)ccc2c(-c3cc(C)cc4c3oc3ccccc34)nccc12 ; CCC(CC)c1cc(F)cc2c(-c3cc(C)cc4c3oc3ccccc34)nccc12 ; CCC(CC)c1cccc2c(-c3cc(C)cc4c3oc3ccccc34)ncc(F)c12 ; CCC(CC)c1cnc(-c2cc(C)cc3c2oc2ccccc23)c2cccc(F)c12]

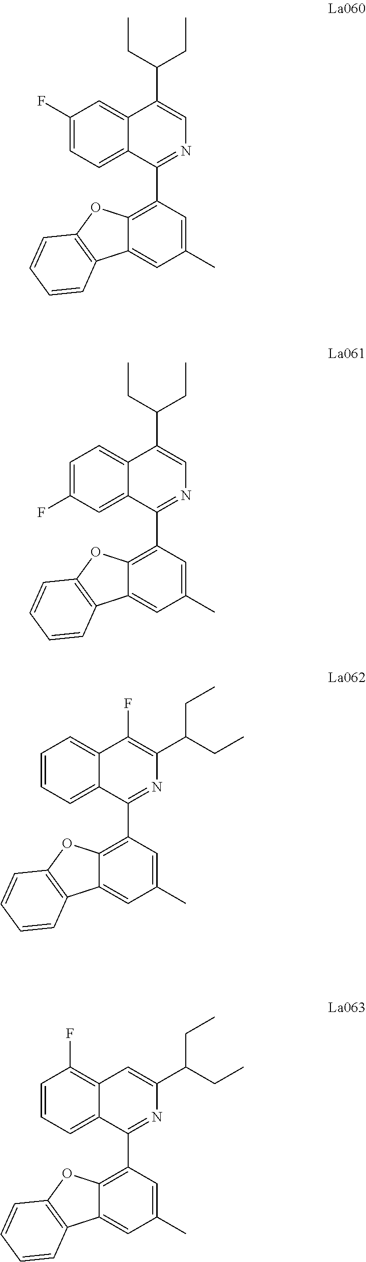 [SMILES: CCC(CC)c1cnc(-c2cc(C)cc3c2oc2ccccc23)c2ccc(F)cc12 ; CCC(CC)c1cnc(-c2cc(C)cc3c2oc2ccccc23)c2cc(F)ccc12 ; CCC(CC)c1nc(-c2cc(C)cc3c2oc2ccccc23)c2ccccc2c1F ; CCC(CC)c1cc2c(F)cccc2c(-c2cc(C)cc3c2oc2ccccc23)n1]

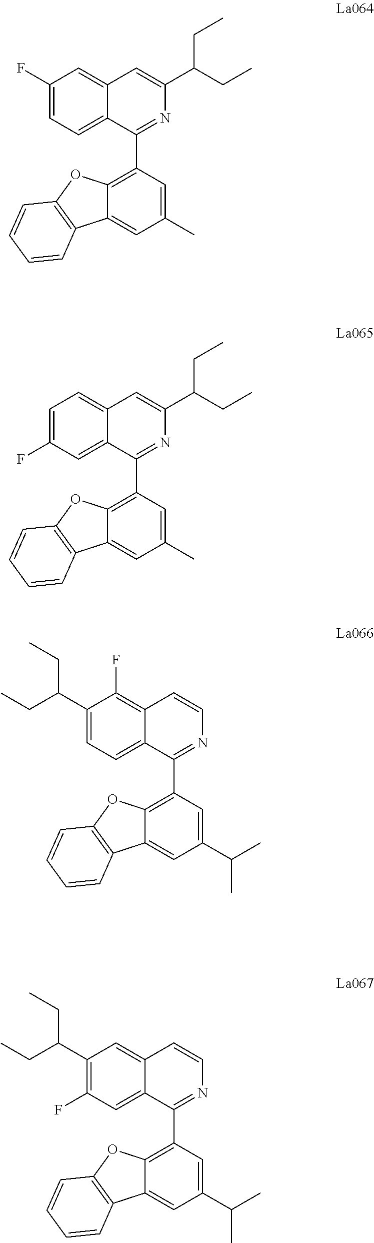

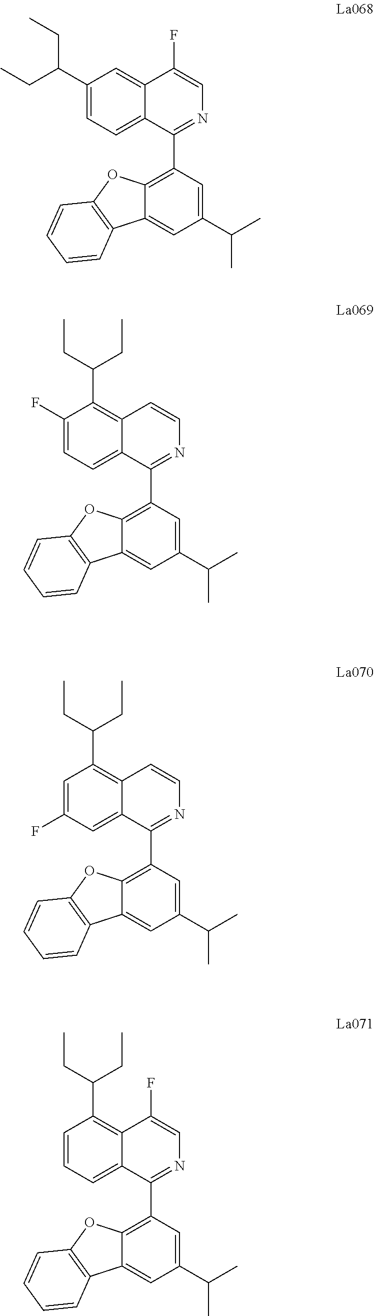 [SMILES: CCC(CC)c1ccc2c(-c3cc(C(C)C)cc4c3oc3ccccc34)ncc(F)c2c1 ; CCC(CC)c1c(F)ccc2c(-c3cc(C(C)C)cc4c3oc3ccccc34)nccc12 ; CCC(CC)c1cc(F)cc2c(-c3cc(C(C)C)cc4c3oc3ccccc34)nccc12 ; CCC(CC)c1cccc2c(-c3cc(C(C)C)cc4c3oc3ccccc34)ncc(F)c12]

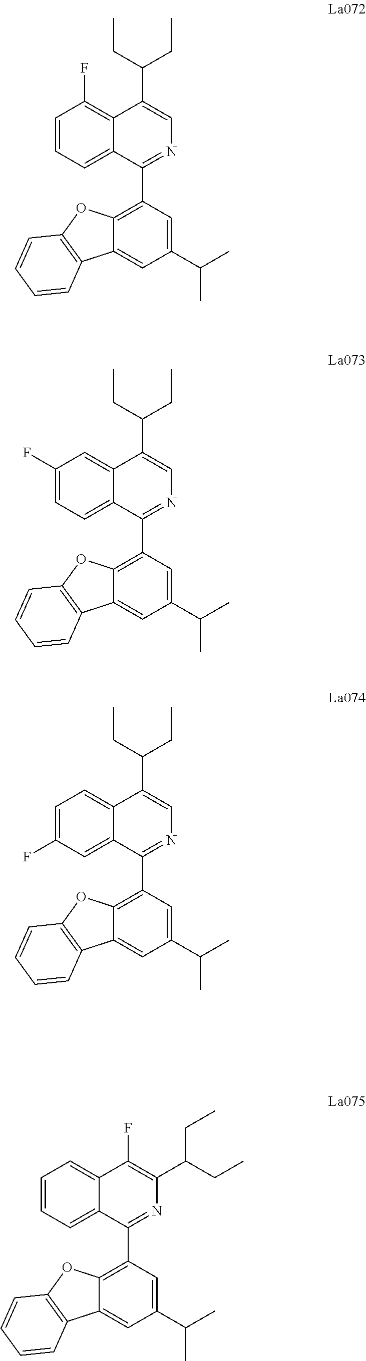 [SMILES: CCC(CC)c1cnc(-c2cc(C(C)C)cc3c2oc2ccccc23)c2cccc(F)c12 ; CCC(CC)c1cnc(-c2cc(C(C)C)cc3c2oc2ccccc23)c2ccc(F)cc12 ; CCC(CC)c1cnc(-c2cc(C(C)C)cc3c2oc2ccccc23)c2cc(F)ccc12 ; CCC(CC)c1nc(-c2cc(C(C)C)cc3c2oc2ccccc23)c2ccccc2c1F]

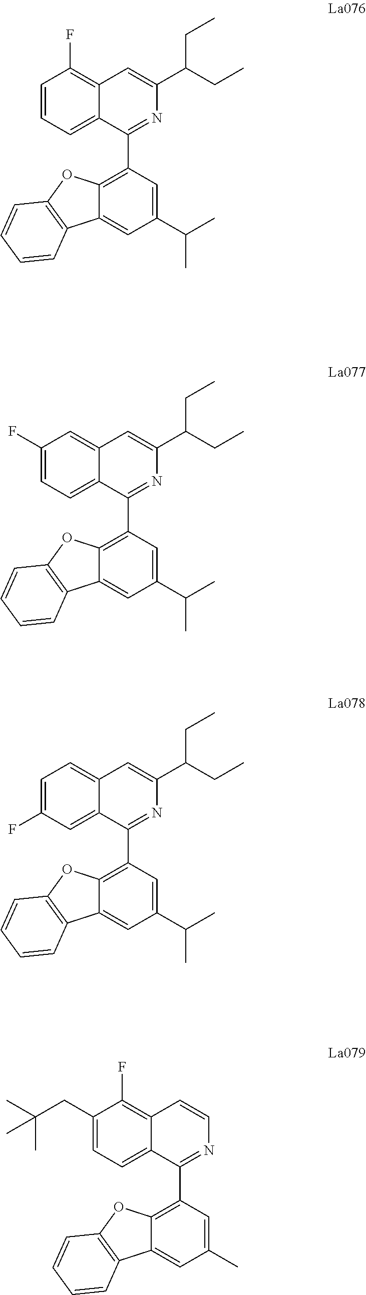

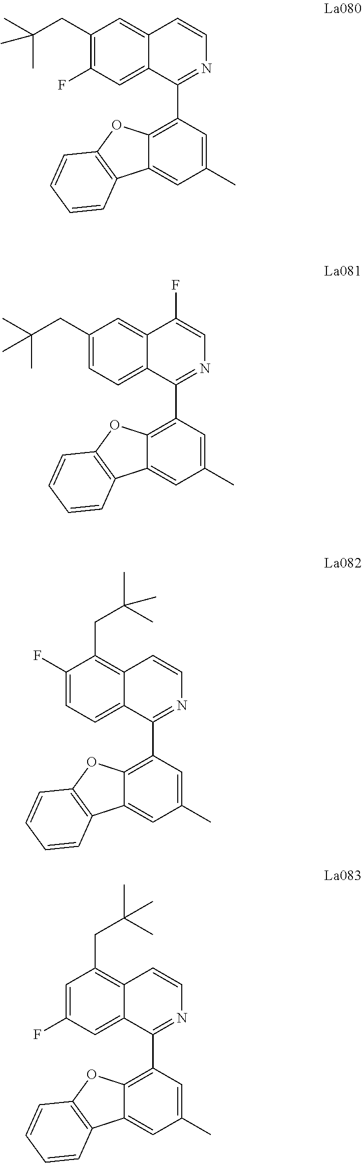

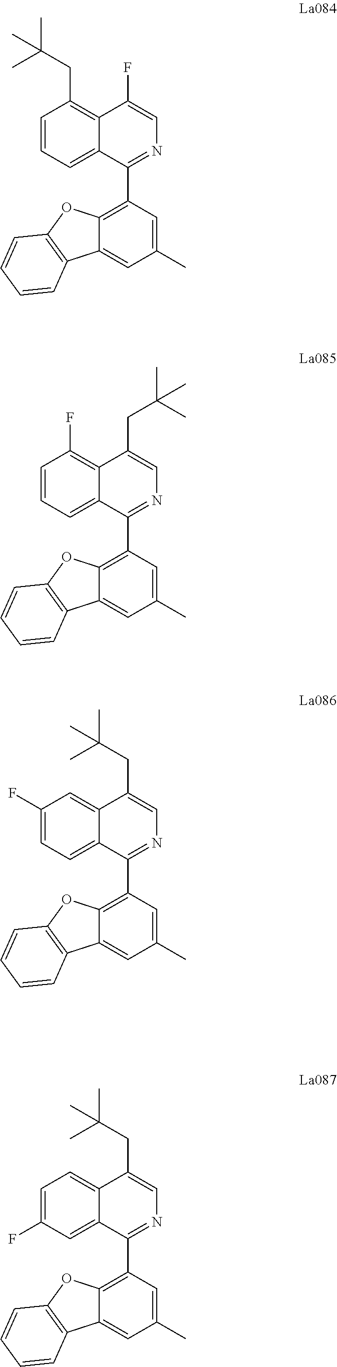 [SMILES: Cc1cc(-c2ncc(F)c3c(CC(C)(C)C)cccc23)c2oc3ccccc3c2c1 ; Cc1cc(-c2ncc(CC(C)(C)C)c3c(F)cccc23)c2oc3ccccc3c2c1 ; Cc1cc(-c2ncc(CC(C)(C)C)c3cc(F)ccc23)c2oc3ccccc3c2c1 ; Cc1cc(-c2ncc(CC(C)(C)C)c3ccc(F)cc23)c2oc3ccccc3c2c1]

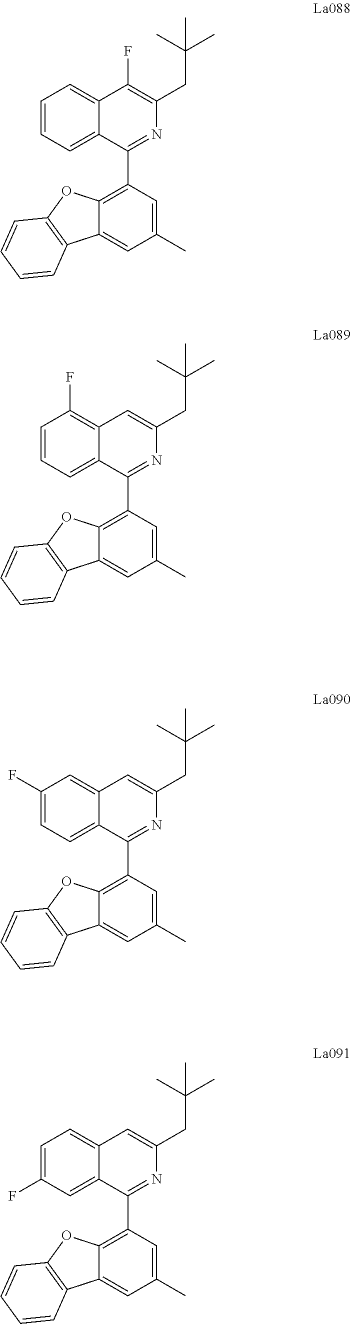 [SMILES: Cc1cc(-c2nc(CC(C)(C)C)c(F)c3ccccc23)c2oc3ccccc3c2c1 ; Cc1cc(-c2nc(CC(C)(C)C)cc3c(F)cccc23)c2oc3ccccc3c2c1 ; Cc1cc(-c2nc(CC(C)(C)C)cc3cc(F)ccc23)c2oc3ccccc3c2c1 ; Cc1cc(-c2nc(CC(C)(C)C)cc3ccc(F)cc23)c2oc3ccccc3c2c1]

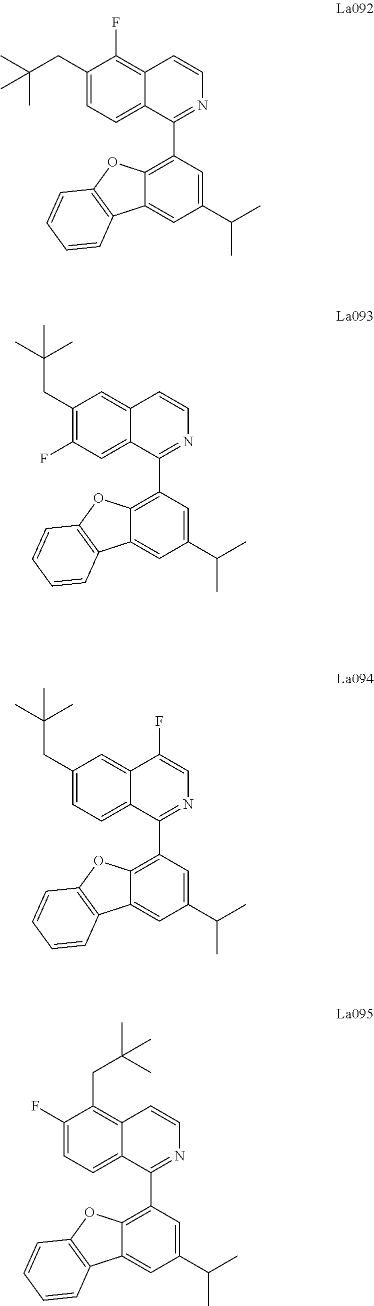

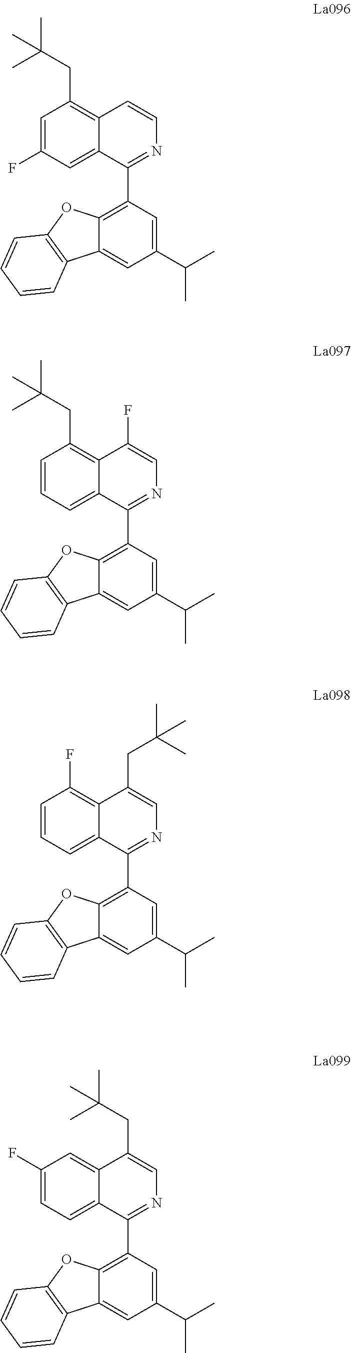 [SMILES: CC(C)c1cc(-c2nccc3c(CC(C)(C)C)cc(F)cc23)c2oc3ccccc3c2c1 ; CC(C)c1cc(-c2ncc(F)c3c(CC(C)(C)C)cccc23)c2oc3ccccc3c2c1 ; CC(C)c1cc(-c2ncc(CC(C)(C)C)c3c(F)cccc23)c2oc3ccccc3c2c1 ; CC(C)c1cc(-c2ncc(CC(C)(C)C)c3cc(F)ccc23)c2oc3ccccc3c2c1]

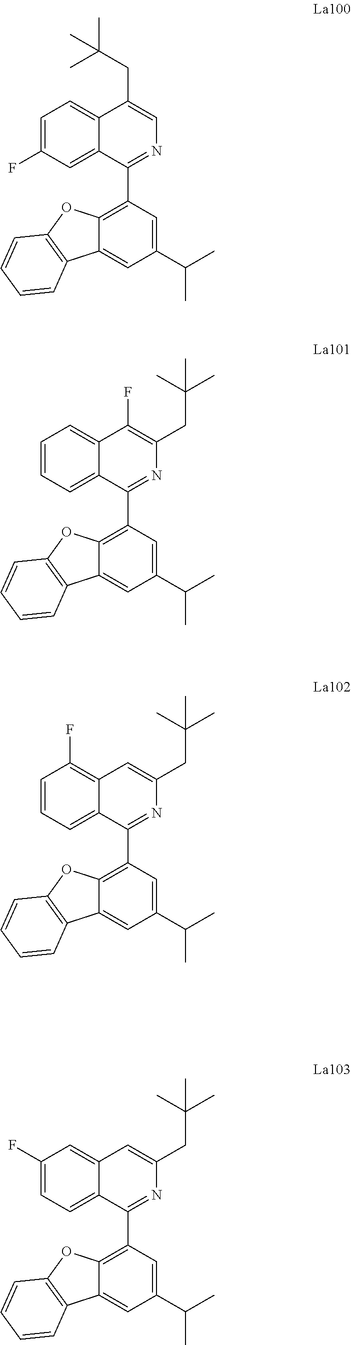 [SMILES: CC(C)c1cc(-c2ncc(CC(C)(C)C)c3ccc(F)cc23)c2oc3ccccc3c2c1 ; CC(C)c1cc(-c2nc(CC(C)(C)C)c(F)c3ccccc23)c2oc3ccccc3c2c1 ; CC(C)c1cc(-c2nc(CC(C)(C)C)cc3c(F)cccc23)c2oc3ccccc3c2c1 ; CC(C)c1cc(-c2nc(CC(C)(C)C)cc3cc(F)ccc23)c2oc3ccccc3c2c1]

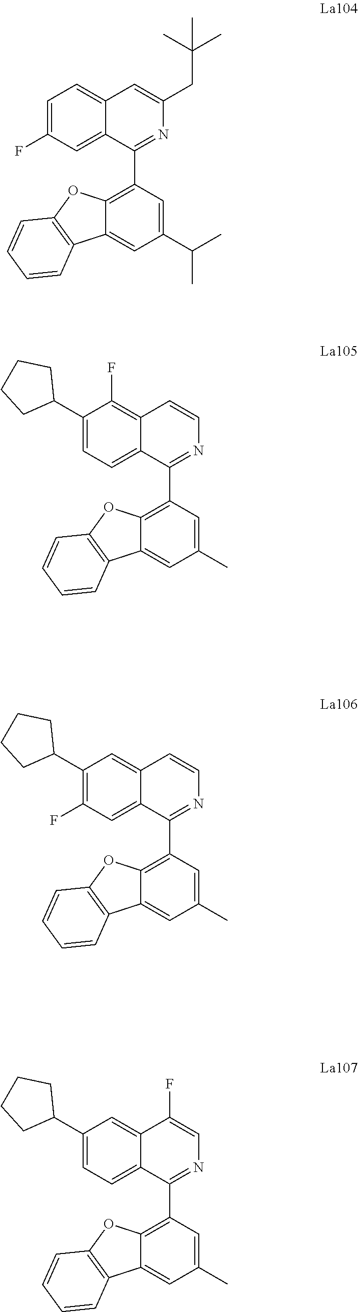 [SMILES: CC(C)c1cc(-c2nc(CC(C)(C)C)cc3ccc(F)cc23)c2oc3ccccc3c2c1 ; Cc1cc(-c2nccc3c(F)c(C4CCCC4)ccc23)c2oc3ccccc3c2c1 ; Cc1cc(-c2nccc3cc(C4CCCC4)c(F)cc23)c2oc3ccccc3c2c1 ; Cc1cc(-c2ncc(F)c3cc(C4CCCC4)ccc23)c2oc3ccccc3c2c1]

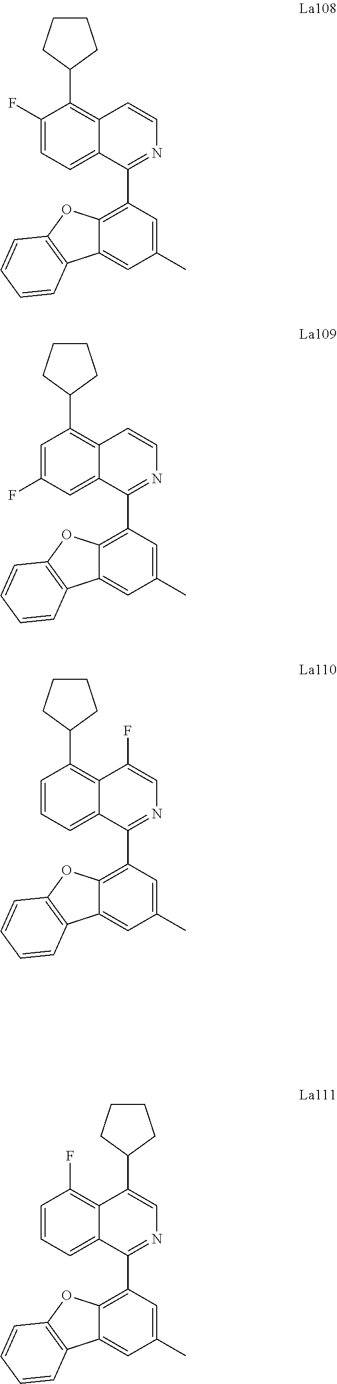 [SMILES: Cc1cc(-c2nccc3c(C4CCCC4)c(F)ccc23)c2oc3ccccc3c2c1 ; Cc1cc(-c2nccc3c(C4CCCC4)cc(F)cc23)c2oc3ccccc3c2c1 ; Cc1cc(-c2ncc(F)c3c(C4CCCC4)cccc23)c2oc3ccccc3c2c1 ; Cc1cc(-c2ncc(C3CCCC3)c3c(F)cccc23)c2oc3ccccc3c2c1]

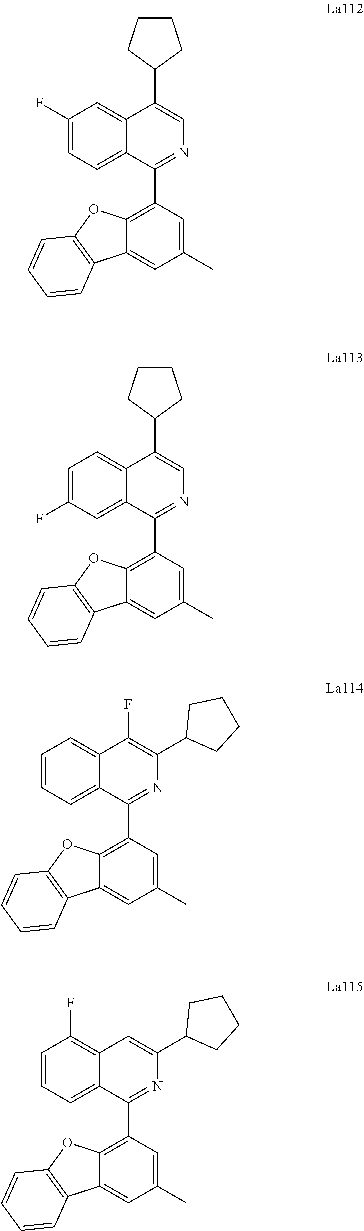 [SMILES: Cc1cc(-c2ncc(C3CCCC3)c3cc(F)ccc23)c2oc3ccccc3c2c1 ; Cc1cc(-c2ncc(C3CCCC3)c3ccc(F)cc23)c2oc3ccccc3c2c1 ; Cc1cc(-c2nc(C3CCCC3)c(F)c3ccccc23)c2oc3ccccc3c2c1 ; Cc1cc(-c2nc(C3CCCC3)cc3c(F)cccc23)c2oc3ccccc3c2c1]

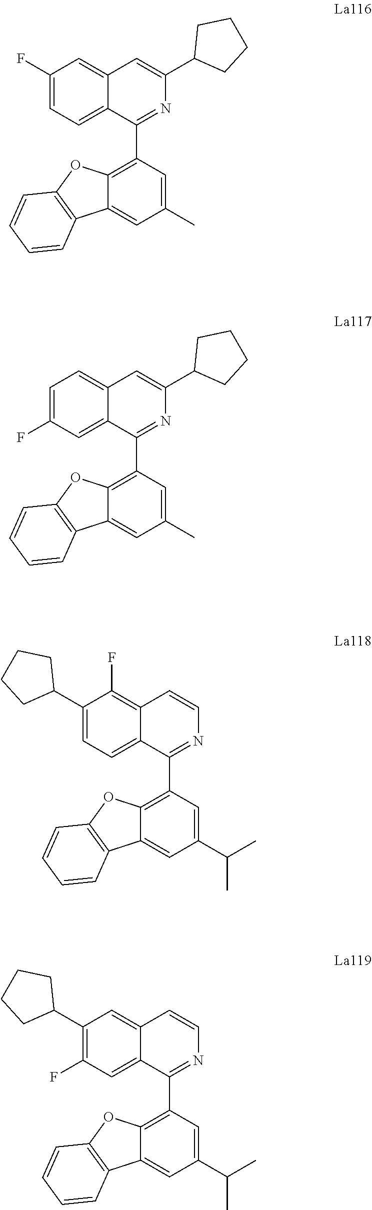 [SMILES: Cc1cc(-c2nc(C3CCCC3)cc3cc(F)ccc23)c2oc3ccccc3c2c1 ; Cc1cc(-c2nc(C3CCCC3)cc3ccc(F)cc23)c2oc3ccccc3c2c1 ; CC(C)c1cc(-c2nccc3c(F)c(C4CCCC4)ccc23)c2oc3ccccc3c2c1 ; CC(C)c1cc(-c2nccc3cc(C4CCCC4)c(F)cc23)c2oc3ccccc3c2c1]

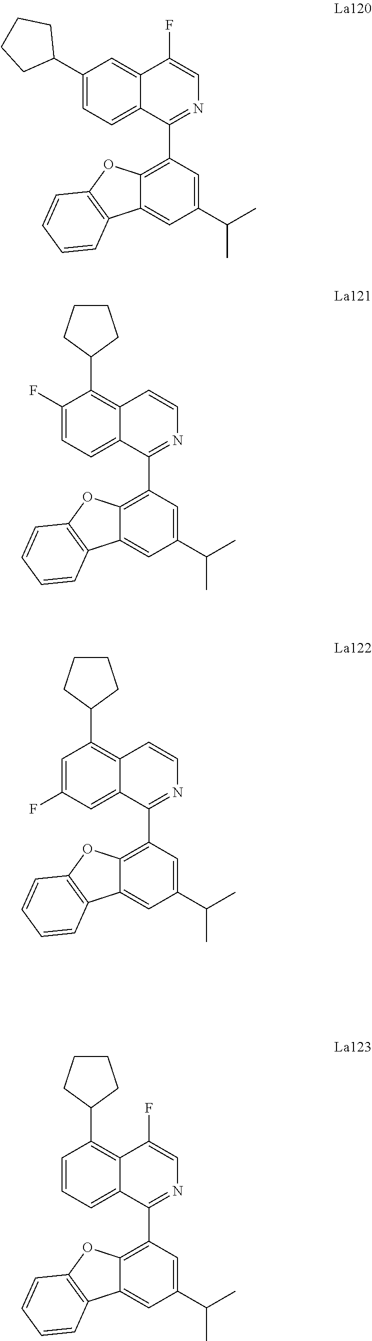 [SMILES: CC(C)c1cc(-c2ncc(F)c3cc(C4CCCC4)ccc23)c2oc3ccccc3c2c1 ; CC(C)c1cc(-c2nccc3c(C4CCCC4)c(F)ccc23)c2oc3ccccc3c2c1 ; CC(C)c1cc(-c2nccc3c(C4CCCC4)cc(F)cc23)c2oc3ccccc3c2c1 ; CC(C)c1cc(-c2ncc(F)c3c(C4CCCC4)cccc23)c2oc3ccccc3c2c1]

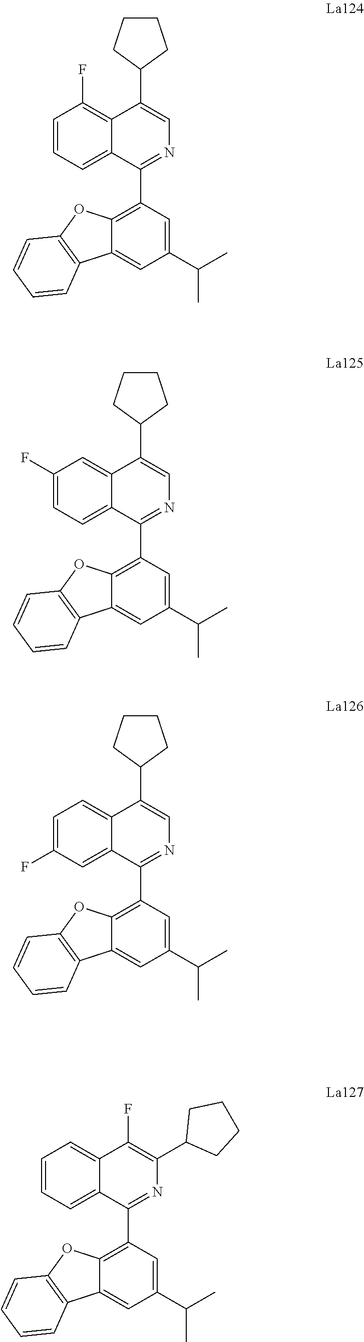 [SMILES: CC(C)c1cc(-c2ncc(C3CCCC3)c3c(F)cccc23)c2oc3ccccc3c2c1 ; CC(C)c1cc(-c2ncc(C3CCCC3)c3cc(F)ccc23)c2oc3ccccc3c2c1 ; CC(C)c1cc(-c2ncc(C3CCCC3)c3ccc(F)cc23)c2oc3ccccc3c2c1 ; CC(C)c1cc(-c2nc(C3CCCC3)c(F)c3ccccc23)c2oc3ccccc3c2c1]

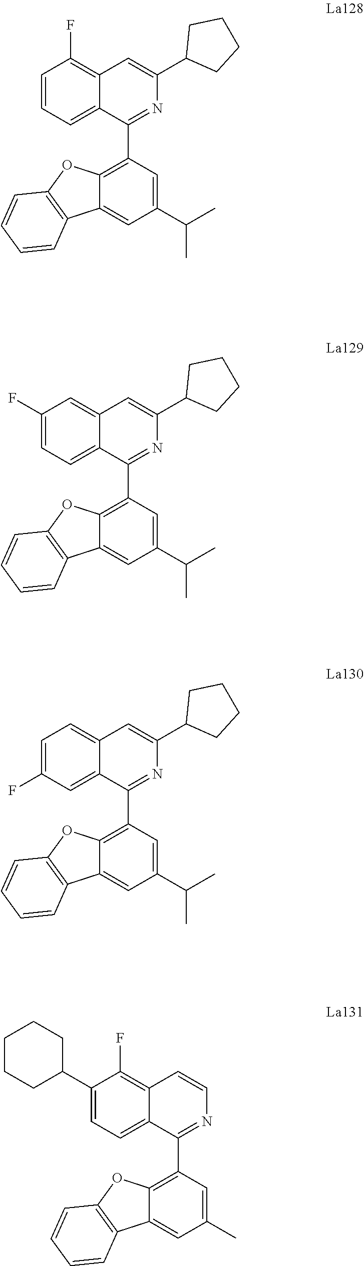 [SMILES: CC(C)c1cc(-c2nc(C3CCCC3)cc3c(F)cccc23)c2oc3ccccc3c2c1 ; CC(C)c1cc(-c2nc(C3CCCC3)cc3cc(F)ccc23)c2oc3ccccc3c2c1 ; CC(C)c1cc(-c2nc(C3CCCC3)cc3ccc(F)cc23)c2oc3ccccc3c2c1 ; Cc1cc(-c2nccc3c(F)c(C4CCCCC4)ccc23)c2oc3ccccc3c2c1]

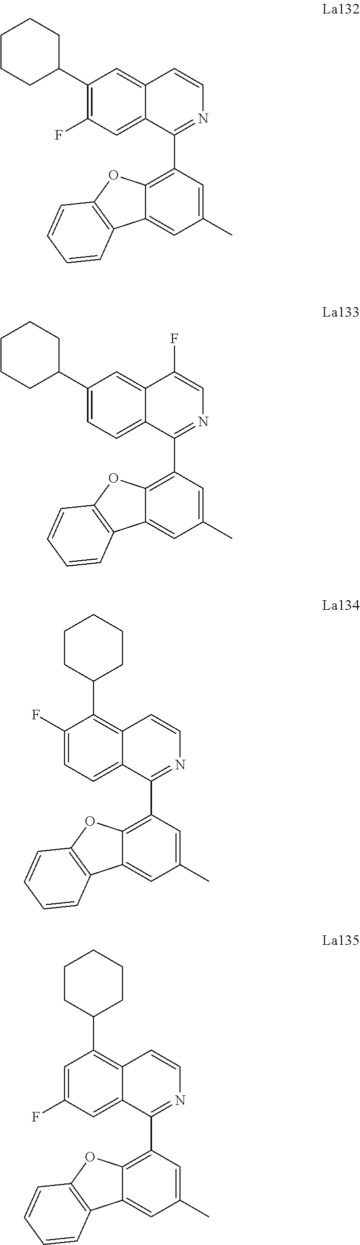 [SMILES: Cc1cc(-c2nccc3cc(C4CCCCC4)c(F)cc23)c2oc3ccccc3c2c1 ; Cc1cc(-c2ncc(F)c3cc(C4CCCCC4)ccc23)c2oc3ccccc3c2c1 ; Cc1cc(-c2nccc3c(C4CCCCC4)c(F)ccc23)c2oc3ccccc3c2c1 ; Cc1cc(-c2nccc3c(C4CCCCC4)cc(F)cc23)c2oc3ccccc3c2c1]

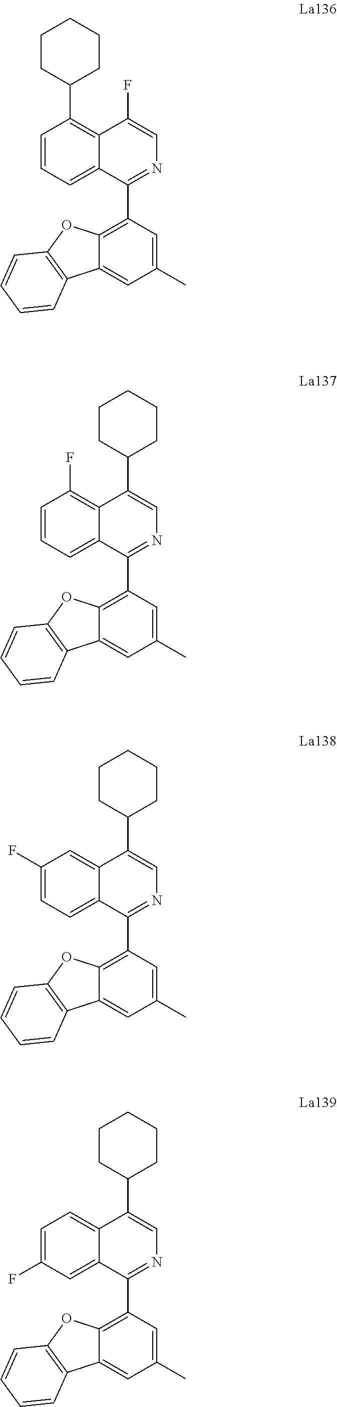 [SMILES: Cc1cc(-c2ncc(F)c3c(C4CCCCC4)cccc23)c2oc3ccccc3c2c1 ; Cc1cc(-c2ncc(C3CCCCC3)c3c(F)cccc23)c2oc3ccccc3c2c1 ; Cc1cc(-c2ncc(C3CCCCC3)c3cc(F)ccc23)c2oc3ccccc3c2c1 ; Cc1cc(-c2ncc(C3CCCCC3)c3ccc(F)cc23)c2oc3ccccc3c2c1]

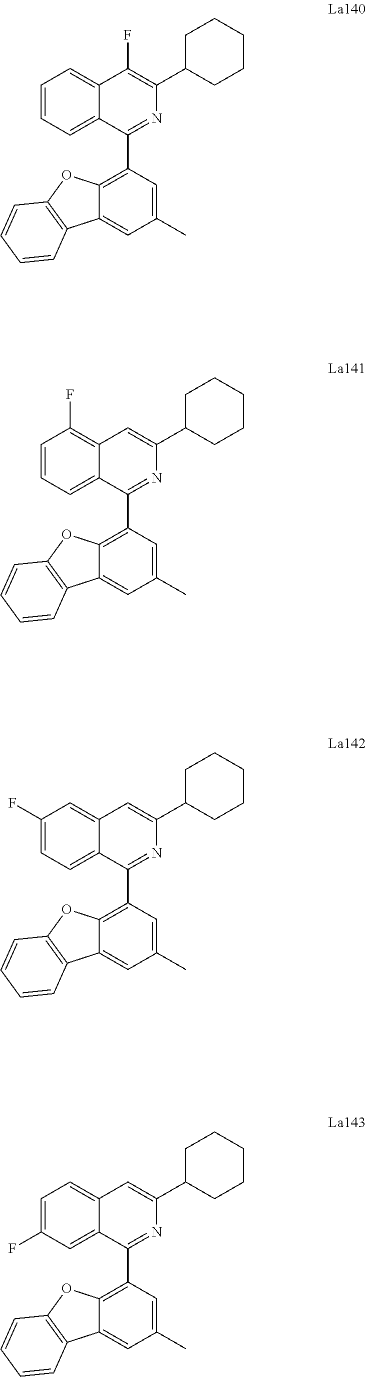 [SMILES: Cc1cc(-c2nc(C3CCCCC3)c(F)c3ccccc23)c2oc3ccccc3c2c1 ; Cc1cc(-c2nc(C3CCCCC3)cc3c(F)cccc23)c2oc3ccccc3c2c1 ; Cc1cc(-c2nc(C3CCCCC3)cc3cc(F)ccc23)c2oc3ccccc3c2c1 ; Cc1cc(-c2nc(C3CCCCC3)cc3ccc(F)cc23)c2oc3ccccc3c2c1]

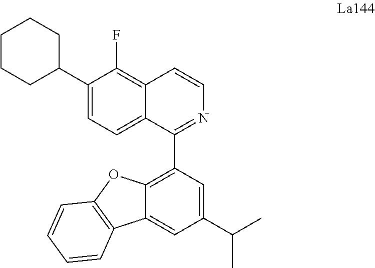 [SMILES: CC(C)c1cc(-c2nccc3c(F)c(C4CCCCC4)ccc23)c2oc3ccccc3c2c1]

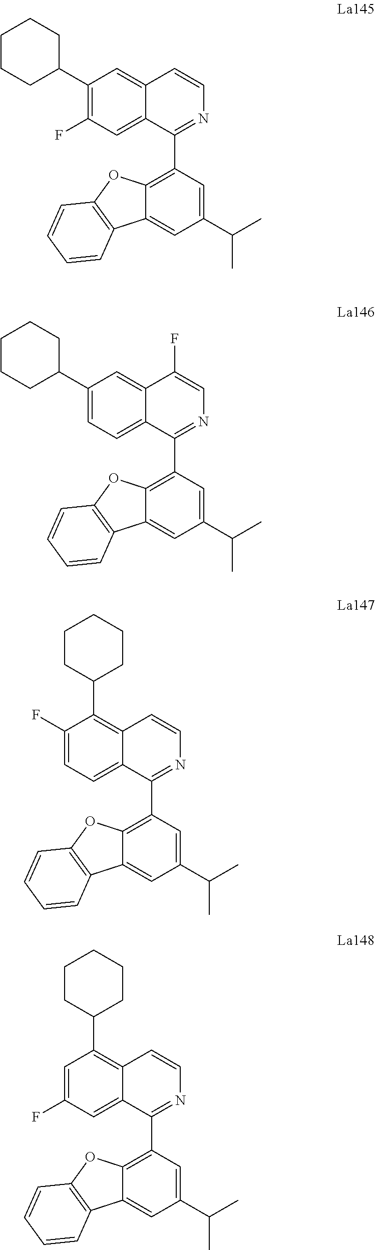 [SMILES: CC(C)c1cc(-c2nccc3cc(C4CCCCC4)c(F)cc23)c2oc3ccccc3c2c1 ; CC(C)c1cc(-c2ncc(F)c3cc(C4CCCCC4)ccc23)c2oc3ccccc3c2c1 ; CC(C)c1cc(-c2nccc3c(C4CCCCC4)c(F)ccc23)c2oc3ccccc3c2c1 ; CC(C)c1cc(-c2nccc3c(C4CCCCC4)cc(F)cc23)c2oc3ccccc3c2c1]

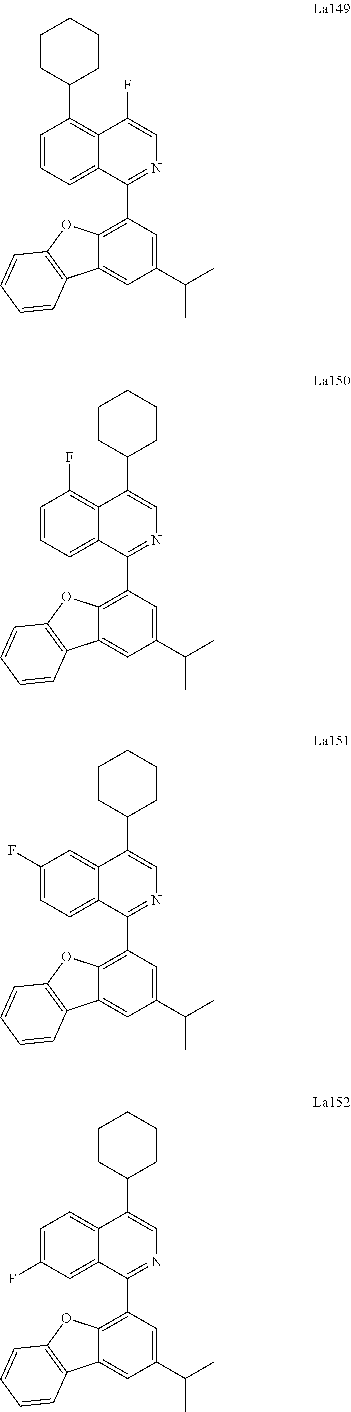 [SMILES: CC(C)c1cc(-c2ncc(F)c3c(C4CCCCC4)cccc23)c2oc3ccccc3c2c1 ; CC(C)c1cc(-c2ncc(C3CCCCC3)c3c(F)cccc23)c2oc3ccccc3c2c1 ; CC(C)c1cc(-c2ncc(C3CCCCC3)c3cc(F)ccc23)c2oc3ccccc3c2c1 ; CC(C)c1cc(-c2ncc(C3CCCCC3)c3ccc(F)cc23)c2oc3ccccc3c2c1]

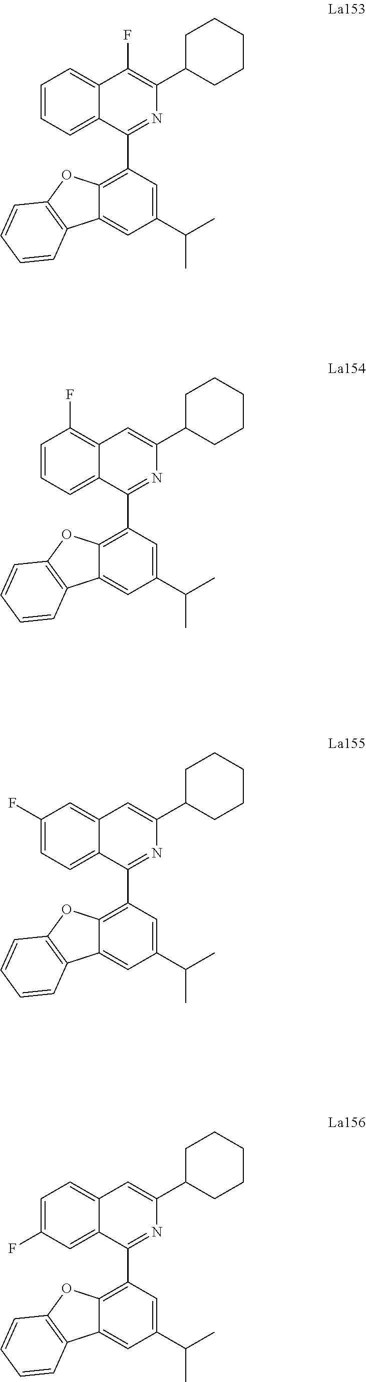 [SMILES: CC(C)c1cc(-c2nc(C3CCCCC3)c(F)c3ccccc23)c2oc3ccccc3c2c1 ; CC(C)c1cc(-c2nc(C3CCCCC3)cc3c(F)cccc23)c2oc3ccccc3c2c1 ; CC(C)c1cc(-c2nc(C3CCCCC3)cc3cc(F)ccc23)c2oc3ccccc3c2c1 ; CC(C)c1cc(-c2nc(C3CCCCC3)cc3ccc(F)cc23)c2oc3ccccc3c2c1]

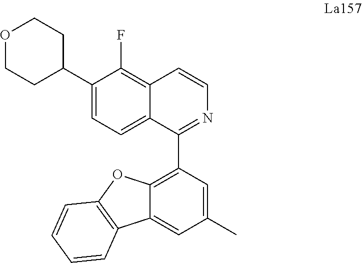 [SMILES: Cc1cc(-c2nccc3c(F)c(C4CCOCC4)ccc23)c2oc3ccccc3c2c1]

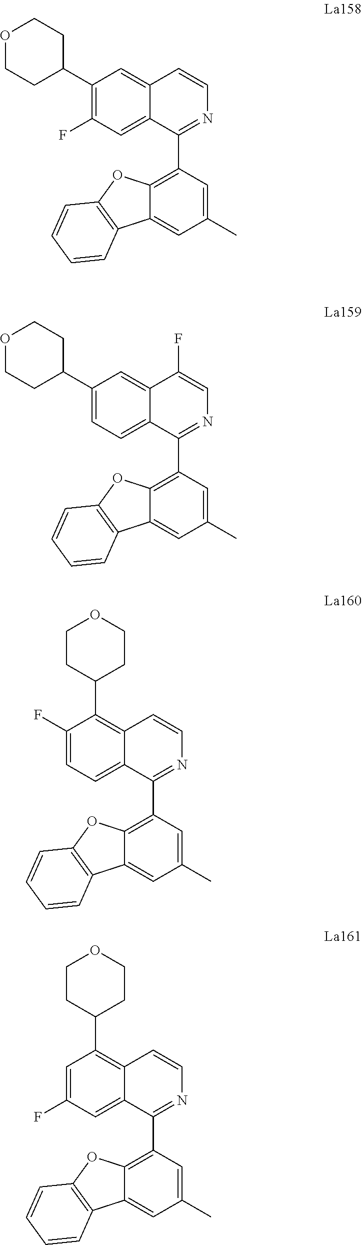 [SMILES: Cc1cc(-c2nccc3cc(C4CCOCC4)c(F)cc23)c2oc3ccccc3c2c1 ; Cc1cc(-c2ncc(F)c3cc(C4CCOCC4)ccc23)c2oc3ccccc3c2c1 ; Cc1cc(-c2nccc3c(C4CCOCC4)c(F)ccc23)c2oc3ccccc3c2c1 ; Cc1cc(-c2nccc3c(C4CCOCC4)cc(F)cc23)c2oc3ccccc3c2c1]

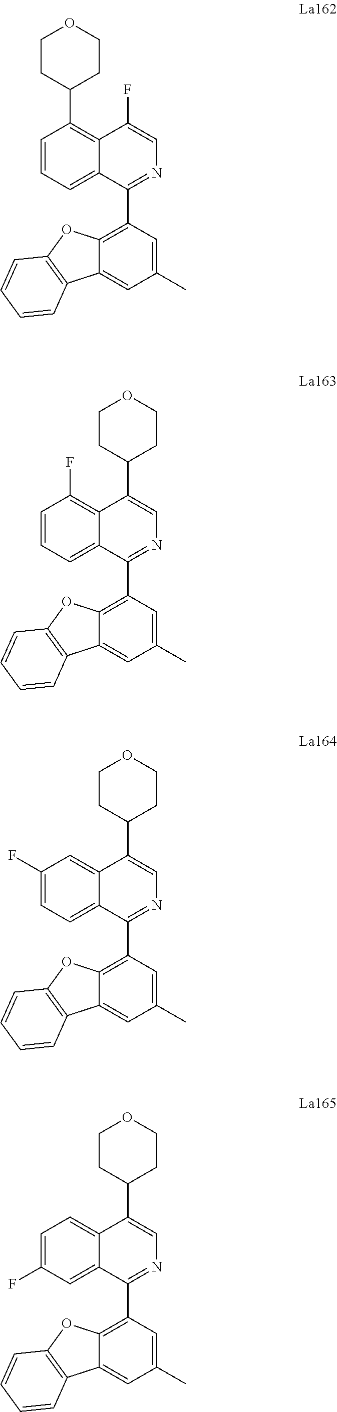 [SMILES: Cc1cc(-c2ncc(F)c3c(C4CCOCC4)cccc23)c2oc3ccccc3c2c1 ; Cc1cc(-c2ncc(C3CCOCC3)c3c(F)cccc23)c2oc3ccccc3c2c1 ; Cc1cc(-c2ncc(C3CCOCC3)c3cc(F)ccc23)c2oc3ccccc3c2c1 ; Cc1cc(-c2ncc(C3CCOCC3)c3ccc(F)cc23)c2oc3ccccc3c2c1]

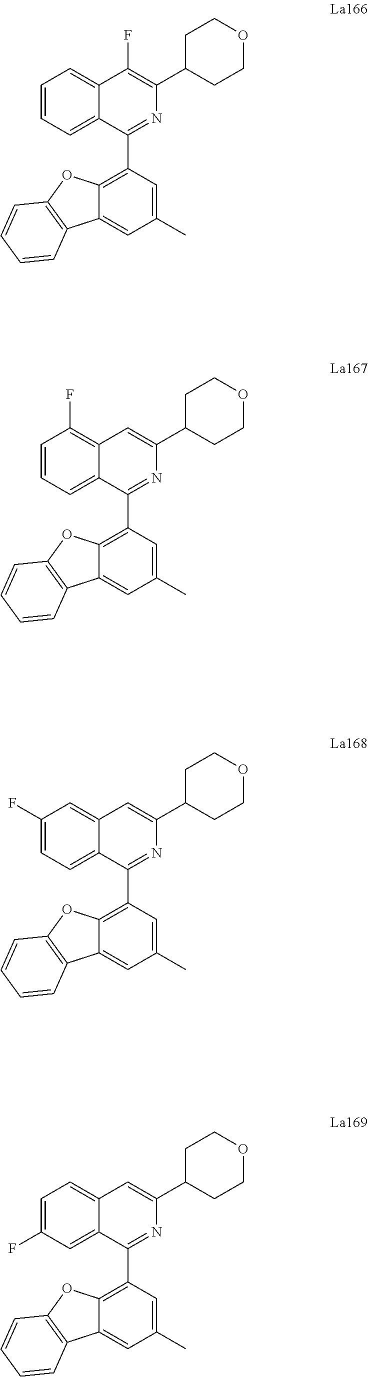 [SMILES: Cc1cc(-c2nc(C3CCOCC3)c(F)c3ccccc23)c2oc3ccccc3c2c1 ; Cc1cc(-c2nc(C3CCOCC3)cc3c(F)cccc23)c2oc3ccccc3c2c1 ; Cc1cc(-c2nc(C3CCOCC3)cc3cc(F)ccc23)c2oc3ccccc3c2c1 ; Cc1cc(-c2nc(C3CCOCC3)cc3ccc(F)cc23)c2oc3ccccc3c2c1]

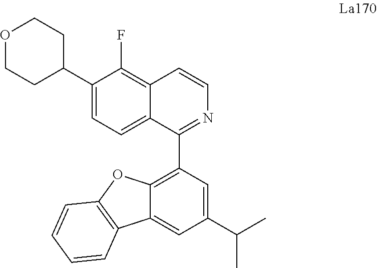 [SMILES: CC(C)c1cc(-c2nccc3c(F)c(C4CCOCC4)ccc23)c2oc3ccccc3c2c1]

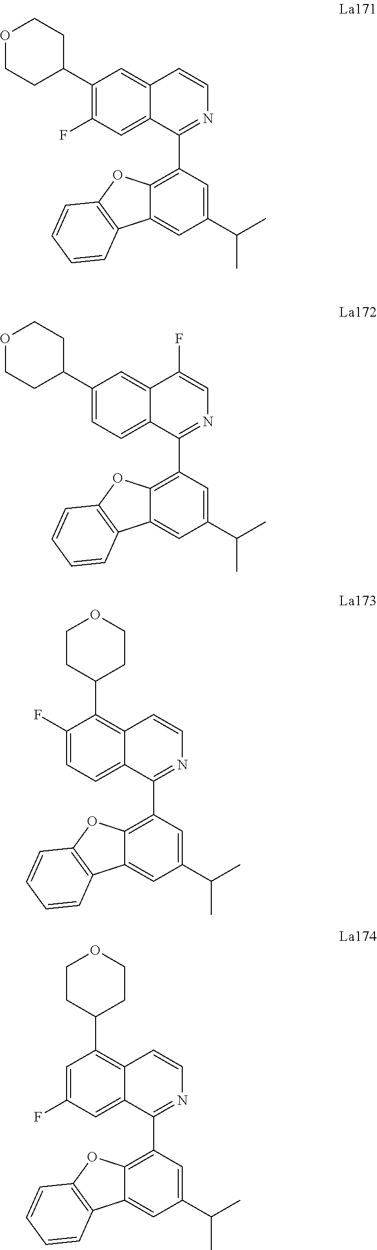 [SMILES: CC(C)c1cc(-c2nccc3cc(C4CCOCC4)c(F)cc23)c2oc3ccccc3c2c1 ; CC(C)c1cc(-c2ncc(F)c3cc(C4CCOCC4)ccc23)c2oc3ccccc3c2c1 ; CC(C)c1cc(-c2nccc3c(C4CCOCC4)c(F)ccc23)c2oc3ccccc3c2c1 ; CC(C)c1cc(-c2nccc3c(C4CCOCC4)cc(F)cc23)c2oc3ccccc3c2c1]

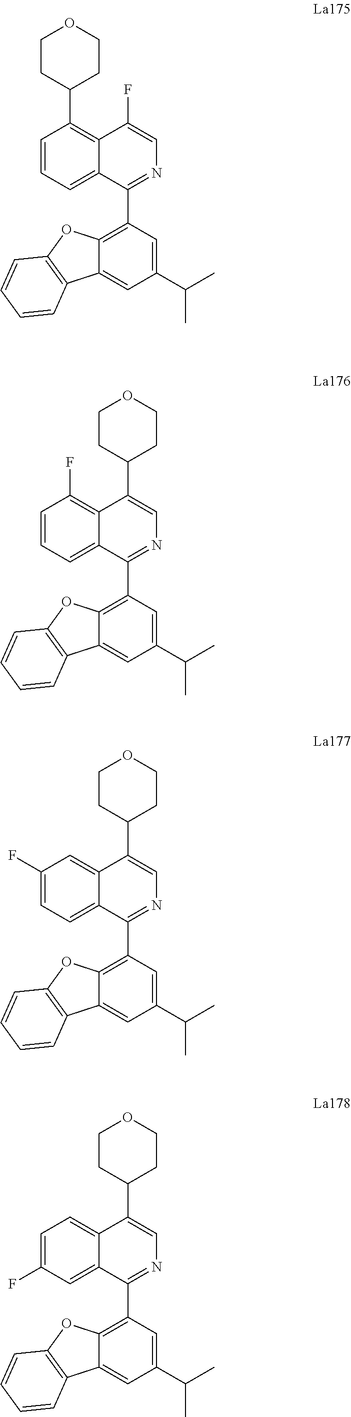 [SMILES: CC(C)c1cc(-c2ncc(F)c3c(C4CCOCC4)cccc23)c2oc3ccccc3c2c1 ; CC(C)c1cc(-c2ncc(C3CCOCC3)c3c(F)cccc23)c2oc3ccccc3c2c1 ; CC(C)c1cc(-c2ncc(C3CCOCC3)c3cc(F)ccc23)c2oc3ccccc3c2c1 ; CC(C)c1cc(-c2ncc(C3CCOCC3)c3ccc(F)cc23)c2oc3ccccc3c2c1]

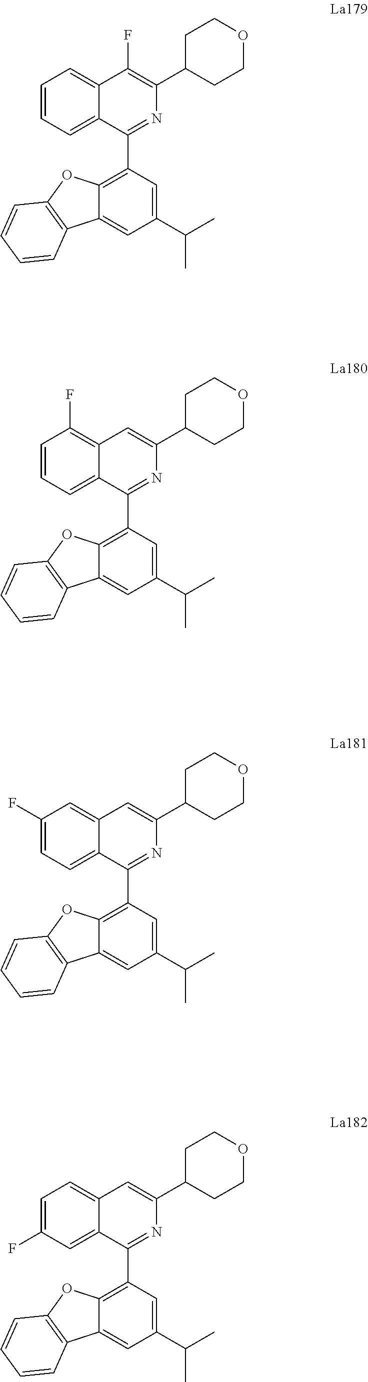 [SMILES: CC(C)c1cc(-c2nc(C3CCOCC3)c(F)c3ccccc23)c2oc3ccccc3c2c1 ; CC(C)c1cc(-c2nc(C3CCOCC3)cc3c(F)cccc23)c2oc3ccccc3c2c1 ; CC(C)c1cc(-c2nc(C3CCOCC3)cc3cc(F)ccc23)c2oc3ccccc3c2c1 ; CC(C)c1cc(-c2nc(C3CCOCC3)cc3ccc(F)cc23)c2oc3ccccc3c2c1]

As a preferred metal complex, the Lb is independently selected from one of the following structural formulas, or corresponding parts or complete deuterides or complete fluorides thereof:

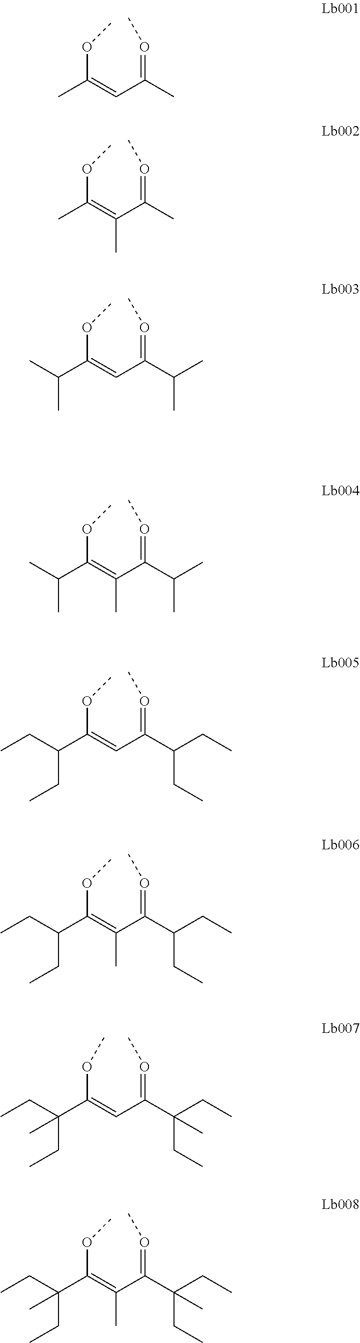

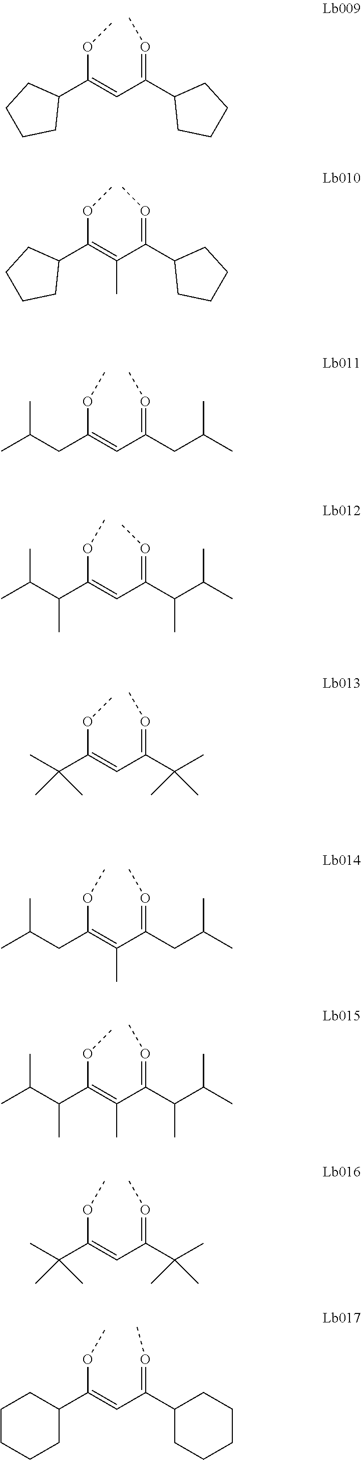

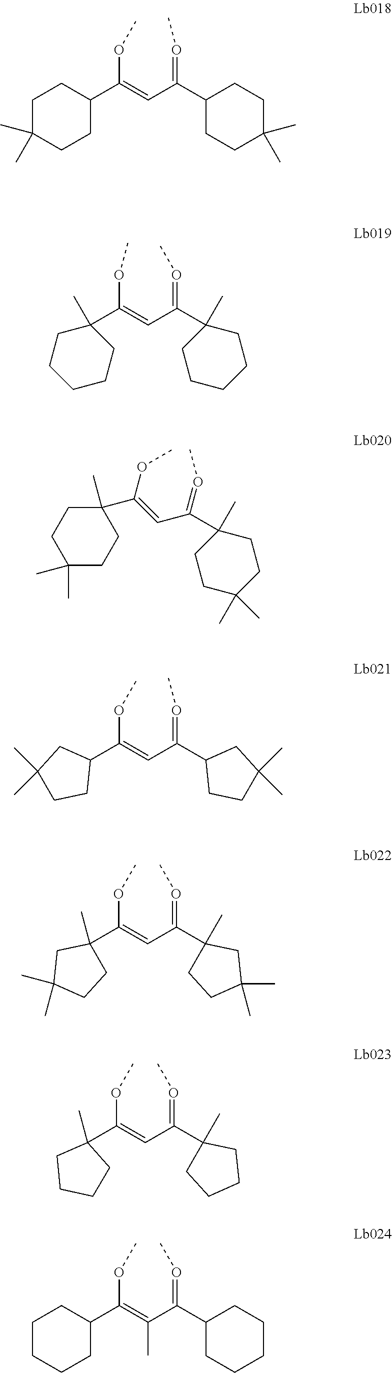

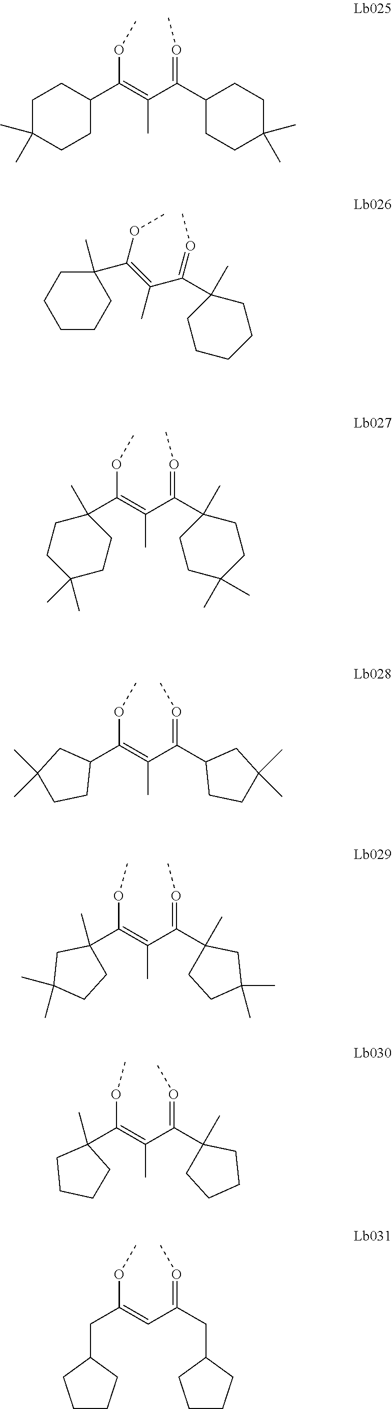

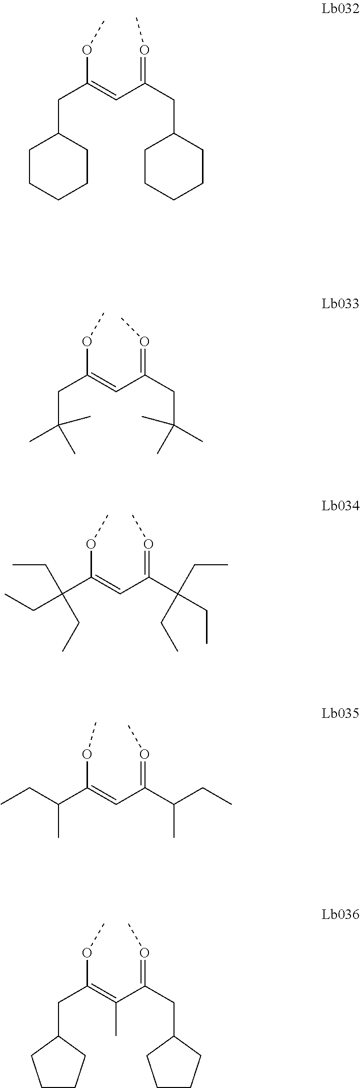

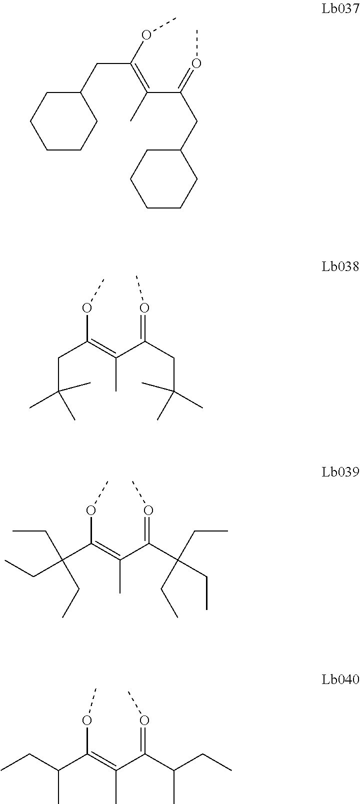

The ligand La has the following structure:

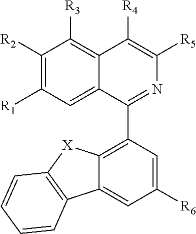

where R₁-R₆ and X are defined as above.

Another invention purpose of the present invention is to provide an electroluminescent device. The electroluminescent device includes a cathode, an anode, and organic layers arranged between the cathode and the anode. At least one of the organic layers includes the metal complex.

The organic layers include a light-emitting layer, and the metal complex is used as a red light-emitting doping material for the light-emitting layer;

or the organic layers include a hole injection layer, and the metal complex is used as a hole injection material in the hole injection layer.

The material of the present invention has the advantages of low sublimation temperature, high optical and electrochemical stability, high color saturation, high luminous efficiency, and long device service life. As a phosphorescent material, the material of the present invention can convert a triplet excited state into light, so that the luminous efficiency of an organic electroluminescent device can be improved, and the energy consumption is reduced.

### BRIEF DESCRIPTION OF DRAWINGS

FIG. 1 is a diagram showing ¹HNMR spectra of a compound La027 of the present invention in a deuterated chloroform solution.

FIG. 2 is a diagram showing ¹HNMR spectra of a compound Ir(La027) 2 (Lb005) of the present invention in a deuterated chloroform solution.

FIG. 3 shows ultraviolet absorption spectra and emission spectra of the compound Ir(La027) 2 (Lb005) of the present invention in a dichloromethane solution.

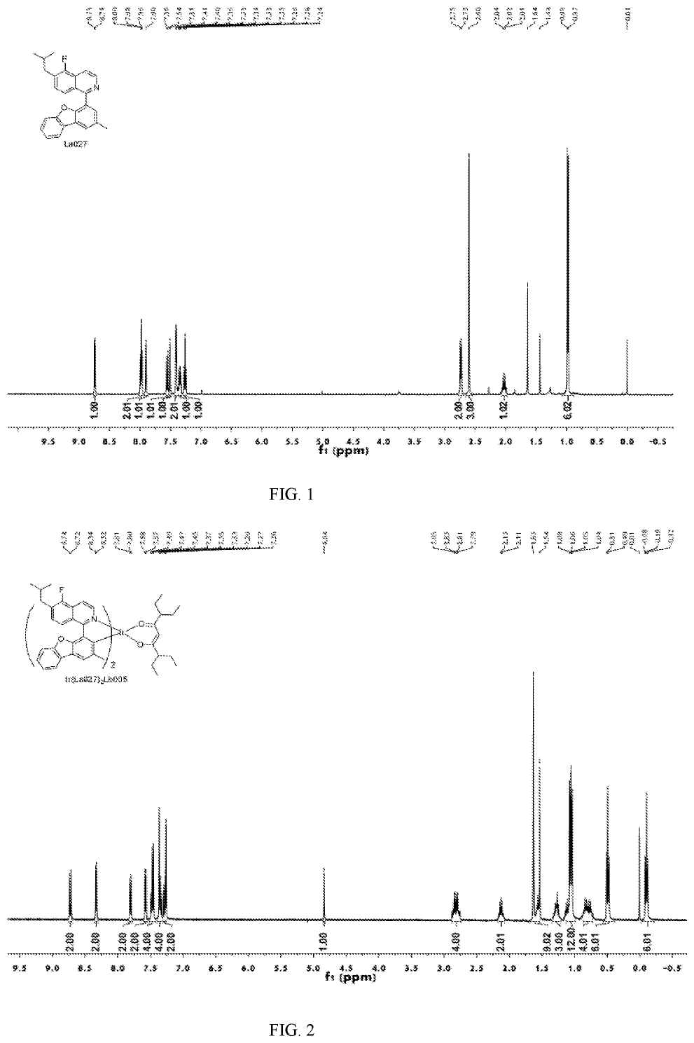

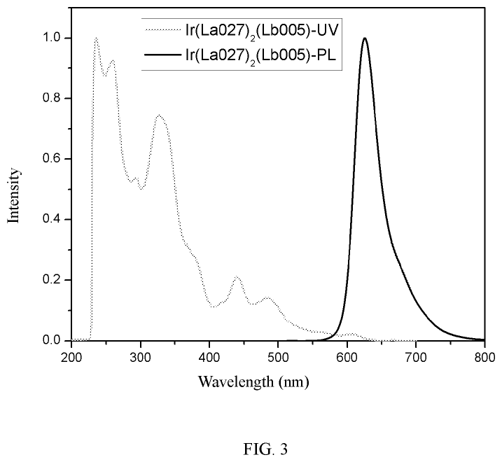

### DETAILED DESCRIPTION OF EMBODIMENTS

A metal complex has a general formula of Ir(La)(Lb)(Lc) and a structural formula as shown in a formula (1):

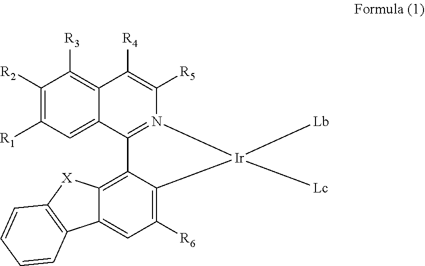

where

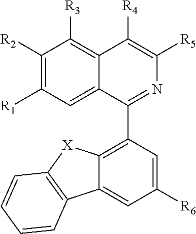

is a ligand La;
X is independently selected from O, S, and Se;
R₁-R₅ are independently selected from hydrogen, deuterium, halogen, substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, substituted or unsubstituted heterocycloalkyl containing 3-20 ring forming carbon atoms, substituted or unsubstituted C₃-C₃₀ alkylsilyl, substituted or unsubstituted C₁-C₁₀ alkoxyl, substituted or unsubstituted C₇-C₃₀ aralkyl, substituted or unsubstituted C₆-C₃₀ aryloxyl, substituted or unsubstituted C₂-C₂₀ alkenyl, substituted or unsubstituted C₂-C₂₀ alkynyl, substituted or unsubstituted C₆-C₃₀ aryl, substituted or unsubstituted C₃-C₃₀ heteroaryl, substituted or unsubstituted C₃-C₃₀ arylsilyl, substituted or unsubstituted C₀-C₂₀ alkylamino, cyano, nitrile, isonitrile, and phosphino;
at least one of the R₁-R₅ is F, and one of the R₁-R₅ is substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, or substituted or unsubstituted heterocycloalkyl containing 3-20 ring forming carbon atoms;
R₆ is substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, or substituted or unsubstituted heterocycloalkyl containing 3-20 ring forming carbon atoms;
the “substituted” refers to substitution with deuterium, F, Cl, Br, C₁-C₄ alkyl, C₁-C₄ alkoxyl, C₃-C₆ cycloalkyl, amino substituted with C₁-C₄ alkyl, cyano, nitrile, isonitrile, or phosphino;
the heteroalkyl or the heteroaryl includes at least one of S, O, and N heteroatoms;
Lb and Lc are both a monoanionic bidentate ligand, any two of the La, the Lb, and the Lc are connected to each other to form a multidentate ligand, or the La, the Lb, and the Lc are connected by a group;
and at least two of the La, the Lb, and the Lc are the same.

As a preferred metal complex, the Lb has a structure as shown in a formula (2):

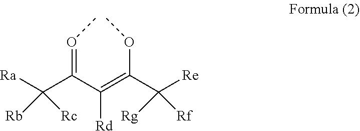

where a dotted line refers to a position connected to metal Ir;
Rₐ-R_{g} are independently selected from hydrogen, deuterium, halogen, substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, and substituted or unsubstituted heterocycloalkyl containing 3-20 ring forming carbon atoms, or any two of Rₐ, R_{b}, and R_{c} are connected to each other to form an aliphatic ring structure, and any two of Rₑ, R_{f}, and R_{g} are connected to each other to form an aliphatic ring structure; and the “substituted” refers to substitution with deuterium, F, Cl, Br, C₁-C₄ alkyl, C₁-C₄ alkoxyl, C₃-C₆ cycloalkyl, amino substituted with C₁-C₄ alkyl, cyano, nitrile, isonitrile, or phosphino.

As a preferred metal complex, the Lc and the La have the same structure, so that a (La)₂Ir(Lb) structure is formed.

The Rₐ, the R_{b}, and the R_{c} are the same as the Rₑ, the R_{f}, and the R_{g}, respectively.

The Rₐ-R_{g} are independently selected from hydrogen, deuterium, halogen, substituted or unsubstituted alkyl containing 1-10 carbon atoms on a main chain, and substituted or unsubstituted cycloalkyl containing 3-20 ring forming carbon atoms, or any two of the Rₐ, the R_{b}, and the R_{c} are connected to each other to form an aliphatic ring structure, and any two of the Rₑ, the R_{f}, and the R_{g} are connected to each other to form an aliphatic ring structure; and the “substituted” refers to substitution with deuterium, F, Cl, Br, C₁-C₄ alkyl, or C₃-C₆ cycloalkyl.

R_{d} is selected from hydrogen, deuterium, halogen, and substituted or unsubstituted alkyl containing 1-10 carbon atoms on a main chain.

As a preferred metal complex, the R₆ is substituted or unsubstituted alkyl containing no more than 4 carbon atoms on a main chain, or substituted or unsubstituted cycloalkyl containing no more than 6 ring forming carbon atoms.

As a preferred metal complex, the F is not positioned at the R₅.

The X is an O atom.

As a preferred metal complex, one of the R₁-R₅ is F, another one is substituted or unsubstituted alkyl containing no more than 4 carbon atoms on a main chain, or substituted or unsubstituted cycloalkyl containing no more than 6 ring forming carbon atoms, and the other three are hydrogen.

As a preferred metal complex, when one of the R₁-R₅ is F, another one is branched alkyl substituted with C₁-C₄ alkyl containing no more than 4 carbon atoms on a main chain.

Examples of various groups of the compound as shown in the formula (1) are described below.

It should be noted that in the specification, “Cₐ-C_{b}” in the term “substituted or unsubstituted Cₐ-C_{b} X group” refers to the number of carbons when the X group is unsubstituted, excluding the number of carbons of a substituent when the X group is substituted.

As a linear or branched alkyl, the C₁-C₁₀ alkyl specifically includes methyl, ethyl, propyl, isopropyl, n-butyl, isobutyl, sec-butyl, tert-butyl, n-pentyl and isomers thereof, n-hexyl and isomers thereof, n-heptyl and isomers thereof, n-octyl and isomers thereof, n-nonyl and isomers thereof, and n-decyl and isomers thereof, preferably methyl, ethyl, propyl, isopropyl, n-butyl, isobutyl, sec-butyl, and tert-butyl, and more preferably propyl, isopropyl, isobutyl, sec-butyl, and tert-butyl.

The C₃-C₂₀ cycloalkyl may include cyclopropyl, cyclobutyl, cyclopentyl, cyclohexyl, 1-adamantyl, 2-adamantyl, 1-norbornyl, and 2-norbornyl, preferably cyclopentyl and cyclohexyl.

The C₂-C₁₀ alkenyl may include vinyl, propenyl, allyl, 1-butadienyl, 2-butadienyl, 1-hexatrienyl, 2-hexatrienyl, and 3-hexatrienyl, preferably propeny and allyl.

As a linear or branched alkyl or cycloalkyl consisting of atoms other than carbon and hydrogen, the C₁-C₁₀ heteroalkyl may include mercaptomethyl methyl, methoxymethyl, ethoxymethyl, tert-butoxyl methyl, N,N-dimethyl methyl, epoxy butyl, epoxy pentyl, and epoxy hexyl, preferably methoxymethyl and epoxy pentyl.

Specific examples of the aryl include phenyl, naphthyl, anthracyl, phenanthryl, tetracenyl, pyrenyl, chrysenyl, benzo[c]phenanthryl, benzo[g]chrysenyl, fluorenyl, benzofluorenyl, dibenzofluorenyl, biphenyl, triphenyl, tetraphenyl, and fluoranthracyl, preferably phenyl and naphthyl.

Specific examples of the heteroaryl may include pyrrolyl, pyrazinyl, pyridyl, pyrimidinyl, triazinyl, indolyl, isoindolyl, imidazolyl, furyl, benzofuryl, isobenzofuryl, dibenzofuryl, dibenzothienyl, azodibenzofuryl, azodibenzothienyl, di azodibenzofuryl, diazodibenzothienyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furzanyl, thienyl, benzothienyl, dihydroacridinyl, az ocarbazolyl, diazocarbazolyl, and quinazolinyl, preferably pyridyl, pyrimidinyl, triazinyl, dibenzofuryl, dibenzothienyl, azodibenzofuryl, azodibenzothienyl, diazodibenzofuryl, diazodibenzothienyl, carbazolyl, azocarbazolyl, and diazocarbazolyl.

The following embodiments are merely described to facilitate the understanding of the technical invention, and should not be considered as specific limitations of the present invention.

All raw materials, solvents and the like involved in the synthesis of compounds in the present invention are purchased from Alfa, Acros, and other suppliers known to persons skilled in the art.

Synthesis of a Ligand La001:

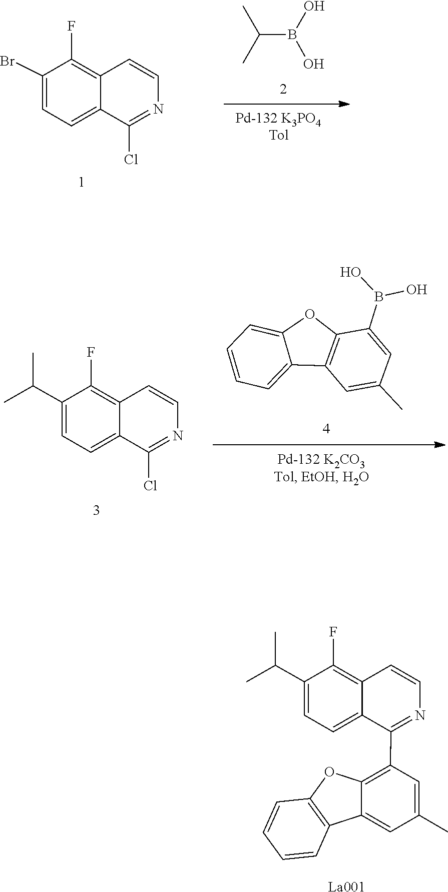

Synthesis of a Compound 3:

A compound 1 (20.00 g, 76.78 mmol, 1.0 eq), a compound 2 (10.12 g, 115.17 mmol, 1.5 eq), dichloro[di-tert-butyl-(4-dimethylaminophenyl)phosphino]palladium (II) (2.72 g, 3.84 mmol, 0.05 eq), anhydrous potassium phosphate (40.74 g, 191.95 mmol, 2.5 eq), and toluene (300 ml) were added into a 1 L three-mouth flask, vacuumization was conducted for the replacement of nitrogen for 3 times, and the above compounds were stirred for a reaction at 100° C. for 4 hours under the protection of nitrogen. According to monitoring by TLC, the compound 1 was completely reacted. After cooling was conducted to room temperature, concentration was conducted under reduced pressure to remove an organic solvent, dichloromethane (150 ml) and deionized water (60 ml) were added for extraction, spin drying was conducted, and separation was conducted by column chromatography (with a mixture of ethyl acetate and n-hexane at a ratio of 1:100 as an eluting agent). Then, concentration was conducted to obtain 9.68 g of a light yellow solid, namely compound 3, with a yield of 56.35%. The mass spectrum was: 224.67 (M+H).

Synthesis of a Compound La001:

The compound 3 (9.20 g, 41.13 mmol, 1.0 eq), a compound 4 (10.23 g, 45.24 mmol, 1.1 eq), dichloro[di-tert-butyl-(4-dimethylaminophenyl)phosphino]palladium (II) (1.46 g, 2.06 mmol, 0.05 eq), potassium carbonate (11.37 g, 82.26 mmol, 2.00 eq), toluene (138 ml), ethanol (46 ml), and deionized water (46 ml) were added into a 500 mL three-mouth flask, vacuumization was conducted for the replacement of nitrogen for 3 times, and the above compounds were stirred for a reaction at 70° C. for 1 hour under the protection of nitrogen. According to monitoring by TLC, the compound 3 was completely reacted. After cooling was conducted to room temperature, concentration was conducted under reduced pressure to remove an organic solvent, dichloromethane (200 ml) and deionized water (80 ml) were added for extraction, spin drying was conducted, and separation was conducted by column chromatography (with a mixture of ethyl acetate and n-hexane at a ratio of 1.5:100 as an eluting agent). Then, concentration was conducted to obtain 9.49 g of a white solid, namely compound La001, with a yield of 62.44%. The mass spectrum was: 370.43 (M+H).

Synthesis of a Compound Ir(La001)₂Lb005:

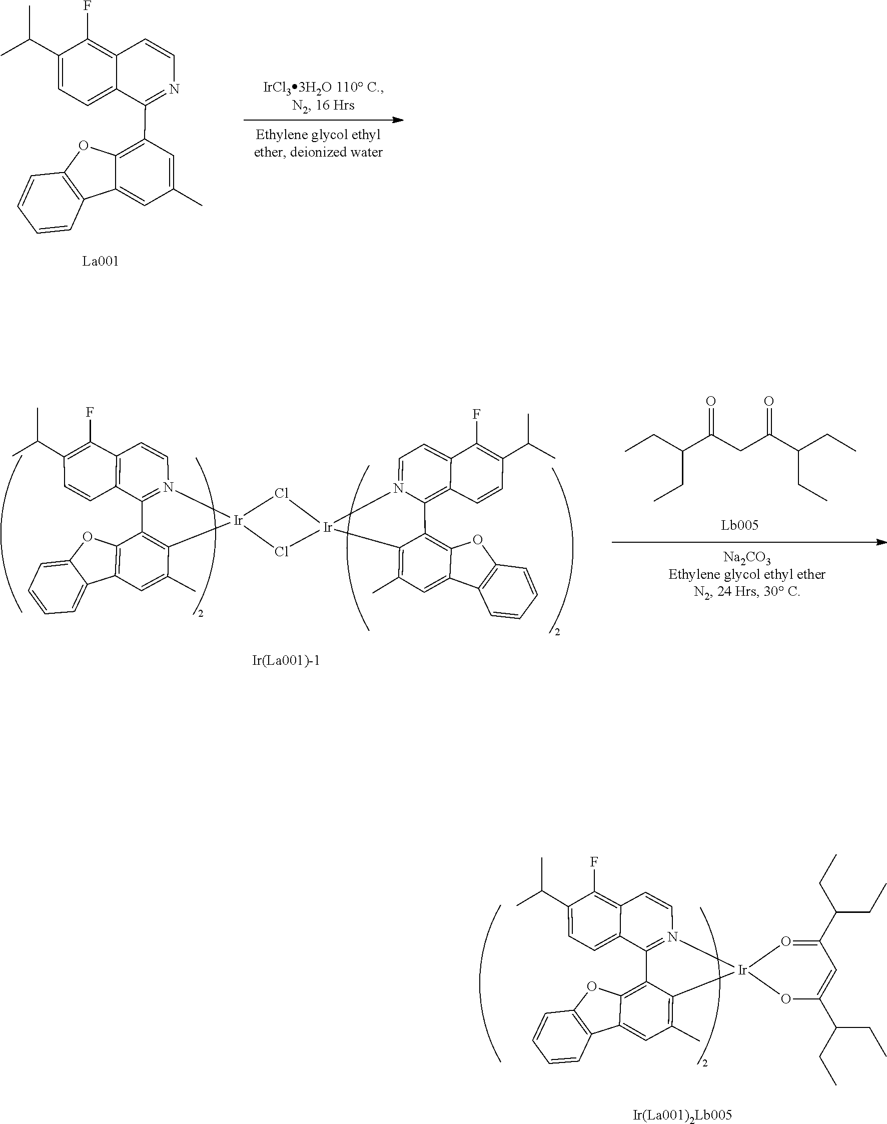

Synthesis of a Compound Ir(La001)-1:

The compound La001 (8.18 g, 22.13 mmol, 3.5 eq) and IrCl₃.3H₂O (2.23 g, 6.32 mmol, 1.0 eq) were placed into a 500 ml round-bottomed one-mouth flask, ethylene glycol ethyl ether (82 ml) and deionized water (27 ml) were added, vacuumization was conducted for replacement for 3 times, and a resulting mixture was stirred at 110° C. for 20 hours under the protection of N₂. After cooling was conducted to room temperature, methanol (90 ml) was added and stirred to precipitate out a solid. Then, the solid was collected after filtration and dried to obtain 5.51 g of a dark red oily substance, namely compound Ir(La001)-1, with a yield of 90.28%. The obtained compound was directly used in the next step without further purification.

Synthesis of a Compound Ir(La001)₂Lb005:

The compound Ir(La001)-1 (5.50 g, 5.7 mmol, 1.0 eq), Lb005 (6.05 g, 28.51 mmol, 5.0 eq), and sodium carbonate (6.04 g, 57.02 mmol, 10.0 eq) were placed in a 250 ml round-bottomed one-mouth flask, ethylene glycol ethyl ether (55 ml) was added, vacuumization was conducted for replacement for 3 times, and a resulting mixture was stirred for a reaction at 30° C. for 19 hours under the protection of N₂. According to monitoring by TLC, the La001-1 was completely reacted. After cooling was conducted to room temperature, 60 ml of methanol was added for beating at room temperature for 2 hours. Suction filtration was conducted, a filter cake was dissolved and clarified in dichloromethane (80 ml) and filtered by silica gel, and a filtrate was washed with deionized water (80 ml) for 3 times. After liquid layering was conducted, an organic phase was collected, concentrated and dried to obtain a dark red solid. Then, the dark red solid was recrystallized with tetrahydrofuran/methanol (a ratio of the product to tetrahydrofuran to methanol was 1 g:6 ml:4 ml) for 3 times, and dried to obtain 2.72 g of a red solid, namely compound Ir(La001)₂Lb005, with a yield of 41.82%. 2.72 g of the crude product Ir(La001)₂Lb005 was sublimated and purified to obtain 1.63 g of sublimated pure Ir(La001)₂Lb005 with a yield of 59.92%. The mass spectrum was: 1141.38 (M+H). ¹H NMR (400 MHz, CDCl₃) δ 8.70 (d, J=8.8 Hz, 2H), 8.31 (d, J=6.5 Hz, 2H), 7.78 (d, J=7.4 Hz, 2H), 7.55 (d, J=6.5 Hz, 2H), 7.50-7.39 (m, 4H), 7.38-7.29 (m, 4H), 7.25 (d, J=7.3 Hz, 2H), 4.84 (s, 1H), 2.16-2.06 (m, 2H), 1.65-1.51 (m, 9H), 1.24 (t, J=11.1 Hz, 3H), 1.10-0.98 (m, 12H), 0.86-0.71 (m, 4H), 0.51 (t, J=7.4 Hz, 6H), −0.11 (t, J=7.3 Hz, 6H).

Synthesis of a Compound La002:

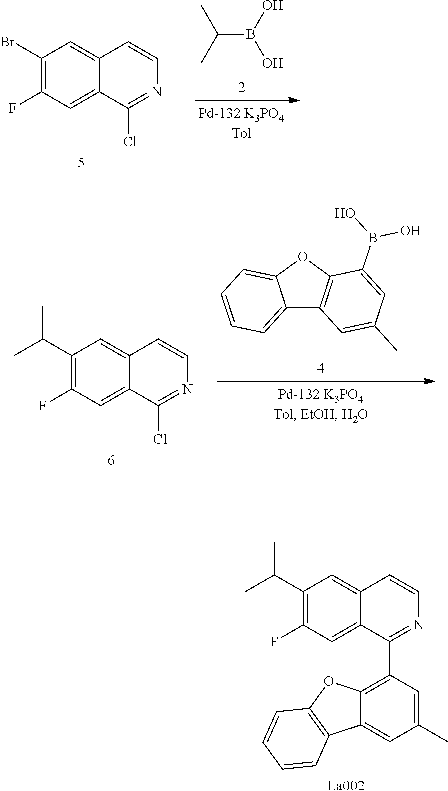

Synthesis of a Compound 6:

With reference to the synthesis and purification methods of the compound 3, only the corresponding raw materials were required to be changed, and a target compound 6 was obtained. The mass spectrum was 224.67 (M+H).

Synthesis of a Compound La002:

With reference to the synthesis and purification methods of the compound La001, only the corresponding raw materials were required to be changed, and a target compound La002 was obtained. The mass spectrum was 370.43 (M+H).

Synthesis of a Compound Ir(La002)₂Lb005:

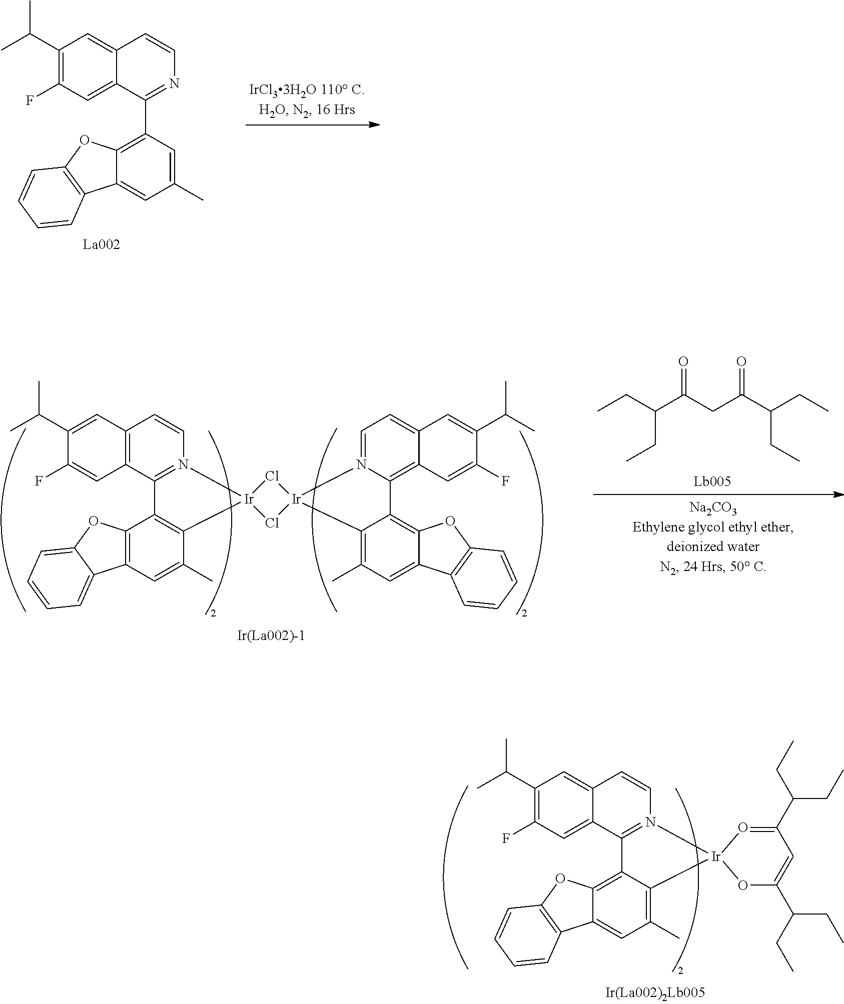

Synthesis of a Compound Ir(La002)-1:

With reference to the synthesis and purification methods of the compound Ir(La001)-1, only the corresponding raw materials were required to be changed, and a compound Ir(La002)-1 was obtained and directly used in the next step without purification.

Synthesis of a Compound Ir(La002)₂Lb005:

With reference to the synthesis and purification methods of the compound Ir(La001)₂Lb005, only the corresponding raw materials were required to be changed, and 2.44 g of a red solid, namely compound Ir(La002)₂Lb005, with a yield of 40.21% was obtained. 2.44 g of the crude product Ir(La002)₂Lb005 was sublimated and purified to obtain 1.56 g of sublimated pure Ir(La002)₂Lb005 with a yield of 59.42%. The mass spectrum was: 1141.38 (M+H). ¹H NMR (400 MHz, CDCl₃) δ 8.62 (d, J=8.5 Hz, 2H), 8.21 (d, J=6.5 Hz, 2H), 7.52 (d, J=7.4 Hz, 2H), 7.42 (d, J=6.5 Hz, 2H), 7.40-7.33 (m, 4H), 7.31-7.26 (m, 4H), 7.23 (d, J=7.3 Hz, 2H), 4.83 (s, 1H), 2.16-2.06 (m, 2H), 1.65-1.51 (m, 9H), 1.24 (t, J=11.1 Hz, 3H), 1.12-0.99 (m, 12H), 0.86-0.71 (m, 4H), 0.52 (t, J=7.4 Hz, 6H), −0.11 (t, J=7.3 Hz, 6H).

Synthesis of a Compound La027:

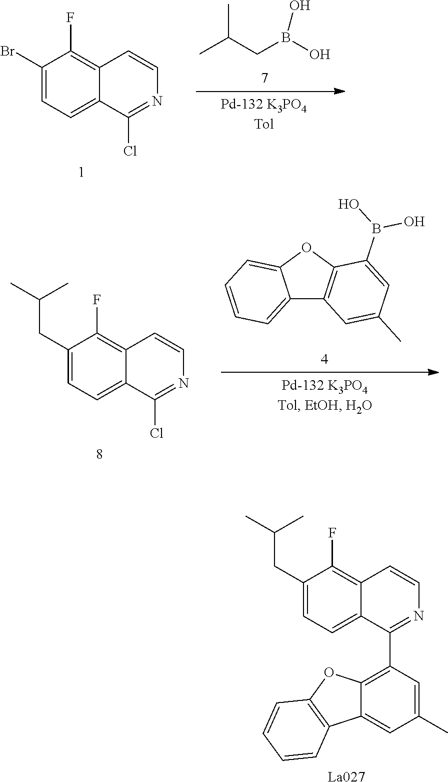

Synthesis of a Compound 8:

With reference to the synthesis and purification methods of the compound 3, only the corresponding raw materials were required to be changed, and a target compound 8 was obtained. The mass spectrum was 238.07 (M+H).

Synthesis of a Compound La027:

With reference to the synthesis and purification methods of the compound La001, only the corresponding raw materials were required to be changed, and a target compound La027 was obtained. The mass spectrum was 384.46 (M+H). ¹H NMR (400 MHz, CDCl₃) δ 8.74 (d, J=5.8 Hz, 1H), 7.98 (t, J=6.3 Hz, 2H), 7.90 (s, 1H), 7.55 (d, J=8.6 Hz, 1H), 7.51 (s, 1H), 7.41 (d, J=3.2 Hz, 2H), 7.37-7.32 (m, 1H), 7.27 (d, J=7.9 Hz, 1H), 2.74 (d, J=7.3 Hz, 2H), 2.60 (s, 3H), 2.07-1.98 (m, 1H), 0.98 (d, J=6.6 Hz, 6H).

Synthesis of a Compound Ir(La027)₂Lb005:

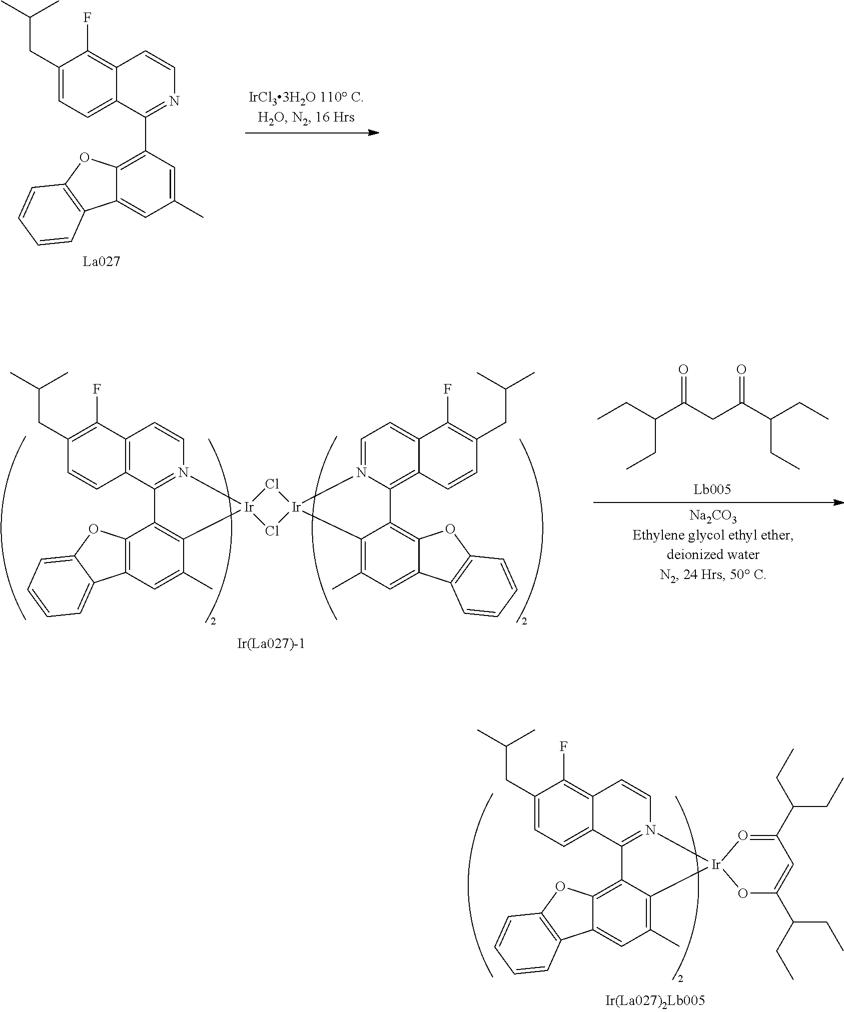

Synthesis of a Compound Ir(La027)-1:

With reference to the synthesis and purification methods of the compound Ir(La001)-1, only the corresponding raw materials were required to be changed, and a compound Ir(La027)-1 was obtained and directly used in the next step without purification.

Synthesis of a Compound Ir(La027)₂Lb005:

With reference to the synthesis and purification methods of the compound Ir(La001)₂Lb005, only the corresponding raw materials were required to be changed, and 2.15 g of a red solid, namely compound Ir(La027)₂Lb005, with a yield of 42.33% was obtained. 2.15 g of the crude product Ir(La027)₂Lb005 was sublimated and purified to obtain 1.32 g of sublimated pure Ir(La027)₂Lb005 with a yield of 61.39%. The mass spectrum was: 1169.44 (M+H). ¹H NMR (400 MHz, CDCl₃) δ 8.73 (d, J=8.8 Hz, 2H), 8.33 (d, J=6.5 Hz, 2H), 7.80 (d, J=7.4 Hz, 2H), 7.57 (d, J=6.5 Hz, 2H), 7.52-7.42 (m, 4H), 7.40-7.31 (m, 4H), 7.28 (d, J=7.3 Hz, 2H), 4.84 (s, 1H), 2.82 (dd, J=15.0, 6.9 Hz, 4H), 2.17-2.07 (m, 2H), 1.68-1.53 (m, 9H), 1.27 (t, J=11.1 Hz, 3H), 1.12-0.99 (m, 12H), 0.87-0.72 (m, 4H), 0.49 (t, J=7.4 Hz, 6H), −0.10 (t, J=7.3 Hz, 6H).

Synthesis of a Compound Ir(La027)₂Lb031:

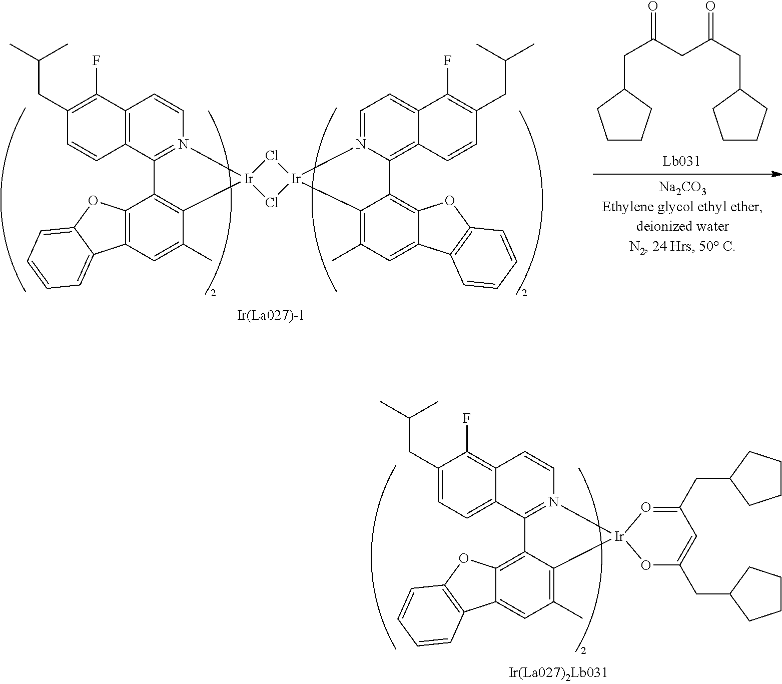

Synthesis of a Compound Ir(La027)₂Lb031:

With reference to the synthesis and purification methods of the compound Ir(La001)₂Lb005, only the corresponding raw materials were required to be changed, and 2.67 g of a red solid, namely compound Ir(La027)₂Lb031, with a yield of 44.68% was obtained. 2.67 g of the crude product Ir(La027)₂Lb031 was sublimated and purified to obtain 1.54 g of sublimated pure Ir(La027)₂Lb031 with a yield of 57.67%. The mass spectrum was: 1193.46 (M+H). ¹H NMR (400 MHz, CDCl₃) 8.73 (d, J=8.8 Hz, 2H), 8.33 (d, J=6.5 Hz, 2H), 7.80 (d, J=7.4 Hz, 2H), 7.57 (d, J=6.5 Hz, 2H), 7.52-7.42 (m, 4H), 7.40-7.31 (m, 4H), 7.28 (d, J=7.3 Hz, 2H), 4.84 (s, 1H), 2.82 (dd, J=15.0, 6.9 Hz, 4H), 2.17-2.07 (m, 2H), 1.92 (s, 6H), 1.83 (d, 4H), 1.78-1.65 (m, 16H), 0.90-0.75 (m, 4H), 0.53 (t, J=7.4 Hz, 4H), 0.13 (t, J=7.3 Hz, 6H).

Synthesis of a Compound La028:

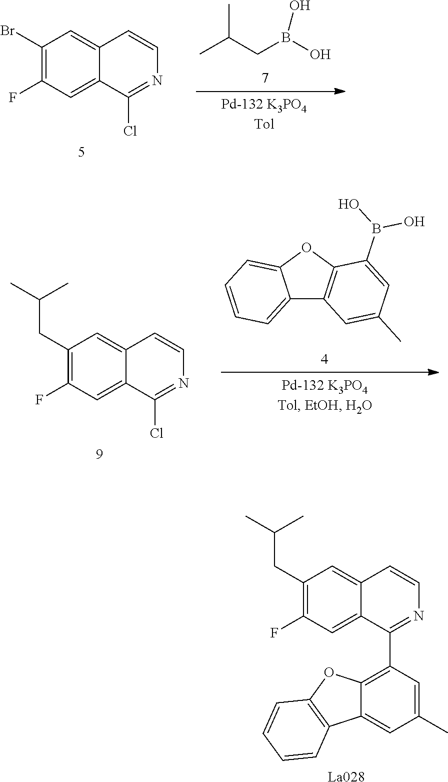

Synthesis of a Compound 9:

With reference to the synthesis and purification methods of the compound 3, only the corresponding raw materials were required to be changed, and a target compound 9 was obtained. The mass spectrum was 238.07 (M+H).

Synthesis of a Compound La028:

With reference to the synthesis and purification methods of the compound La001, only the corresponding raw materials were required to be changed, and a target compound La028 was obtained. The mass spectrum was 384.46 (M+H).

Synthesis of a Compound Ir(La028)₂Lb005:

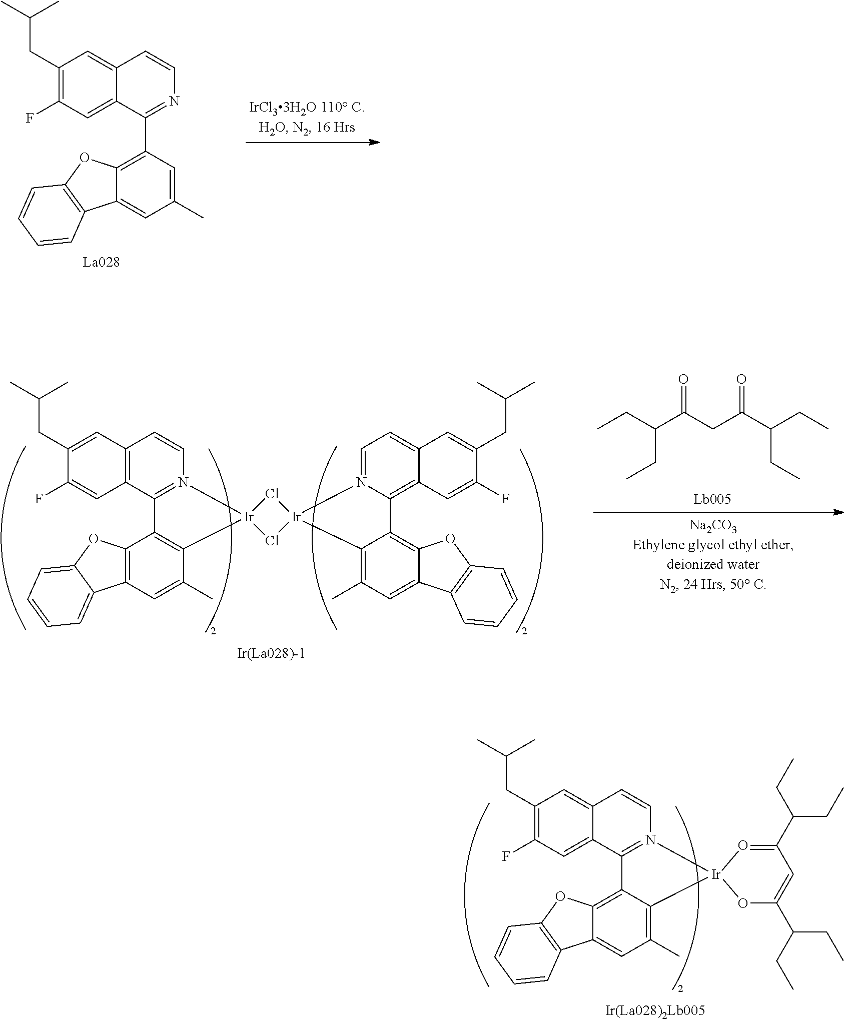

Synthesis of a Compound Ir(La028)-1:

With reference to the synthesis and purification methods of the compound Ir(La001)-1, only the corresponding raw materials were required to be changed, and a compound Ir(La028)-1 was obtained and directly used in the next step without purification.

Synthesis of a Compound Ir(La028)₂Lb005:

With reference to the synthesis and purification methods of the compound Ir(La001)₂Lb005, only the corresponding raw materials were required to be changed, and 1.96 g of a red solid, namely compound Ir(La028)₂Lb005, with a yield of 38.77% was obtained. 1.96 g of the crude product Ir(La028)₂Lb005 was sublimated and purified to obtain 1.14 g of sublimated pure Ir(La028)₂Lb005 with a yield of 58.16%. The mass spectrum was: 1169.44 (M+H). ¹H NMR (400 MHz, CDCl₃) δ 8.77 (d, J=8.6 Hz, 2H), 8.35 (d, J=6.6 Hz, 2H), 7.82 (d, J=7.4 Hz, 2H), 7.59 (d, J=6.5 Hz, 2H), 7.54-7.44 (m, 4H), 7.43-7.34 (m, 4H), 7.31 (d, J=7.3 Hz, 2H), 4.83 (s, 1H), 2.83 (dd, J=15.1, 6.7 Hz, 4H), 2.19-2.08 (m, 2H), 1.68-1.55 (m, 9H), 1.28 (t, J=11.3 Hz, 3H), 1.13-0.99 (m, 12H), 0.88-0.73 (m, 4H), 0.51 (t, J=7.4 Hz, 6H), −0.09 (t, J=7.3 Hz, 6H).

Synthesis of a Compound La037:

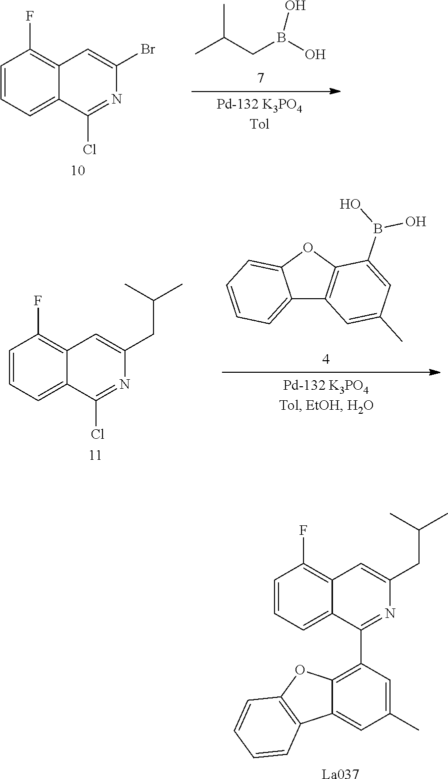

Synthesis of a Compound 11:

With reference to the synthesis and purification methods of the compound 3, only the corresponding raw materials were required to be changed, and a target compound 11 was obtained. The mass spectrum was 238.07 (M+H).

Synthesis of a Compound La037:

With reference to the synthesis and purification methods of the compound La001, only the corresponding raw materials were required to be changed, and a target compound La037 was obtained. The mass spectrum was 384.46 (M+H).

Synthesis of a Compound Ir(La037)₂Lb005:

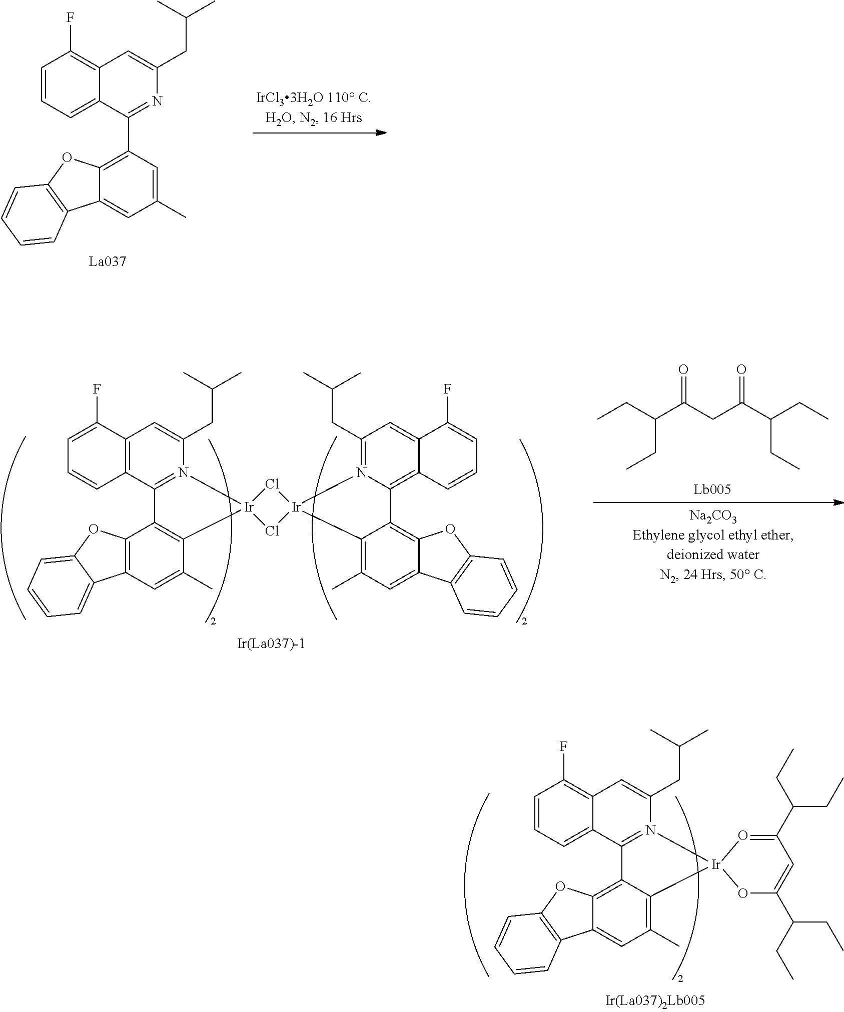

Synthesis of a Compound Ir(La037)-1:

With reference to the synthesis and purification methods of the compound Ir(La001)-1, only the corresponding raw materials were required to be changed, and a compound Ir(La037)-1 was obtained and directly used in the next step without purification.

Synthesis of a Compound Ir(La037)₂Lb005:

With reference to the synthesis and purification methods of the compound Ir(La001)₂Lb005, only the corresponding raw materials were required to be changed, and 1.96 g of a red solid, namely compound Ir(La037)₂Lb005, with a yield of 38.77% was obtained. 1.96 g of the crude product Ir(La037)₂Lb005 was sublimated and purified to obtain 1.14 g of sublimated pure Ir(La037)₂Lb005 with a yield of 58.16%. The mass spectrum was: 1169.44 (M+H). ¹H NMR (400 MHz, CDCl₃) δ 8.71 (d, J=8.6 Hz, 2H), 8.29 (d, J=6.6 Hz, 2H), 7.76 (d, J=7.4 Hz, 2H), 7.54 (d, J=6.5 Hz, 2H), 7.50-7.39 (m, 4H), 7.37-7.27 (m, 4H), 7.22 (d, J=7.3 Hz, 2H), 4.83 (s, 1H), 2.83 (dd, J=15.1, 6.7 Hz, 4H), 2.19-2.08 (m, 2H), 1.68-1.55 (m, 9H), 1.28 (t, J=11.3 Hz, 3H), 1.13-0.99 (m, 12H), 0.88-0.73 (m, 4H), 0.51 (t, J=7.4 Hz, 6H), −0.09 (t, J=7.3 Hz, 6H).

Synthesis of a Compound La080:

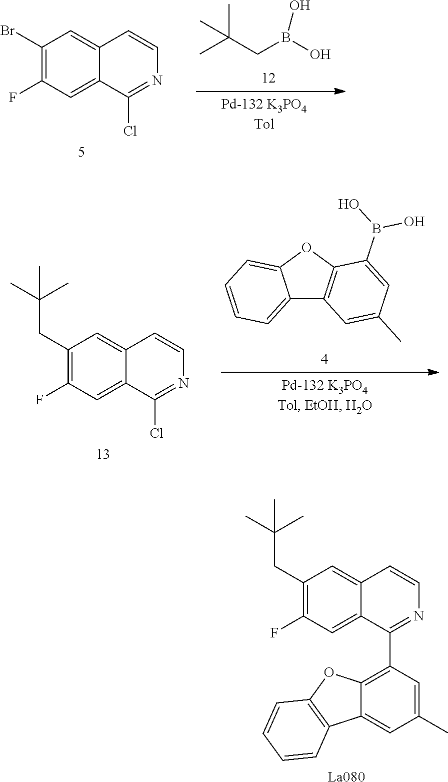

Synthesis of a Compound 13:

With reference to the synthesis and purification methods of the compound 3, only the corresponding raw materials were required to be changed, and a target compound 13 was obtained. The mass spectrum was 252.73 (M+H).

Synthesis of a Compound La080:

With reference to the synthesis and purification methods of the compound La001, only the corresponding raw materials were required to be changed, and a target compound La080 was obtained. The mass spectrum was 398.48 (M+H).

Synthesis of a Compound Ir(La080)₂Lb005:

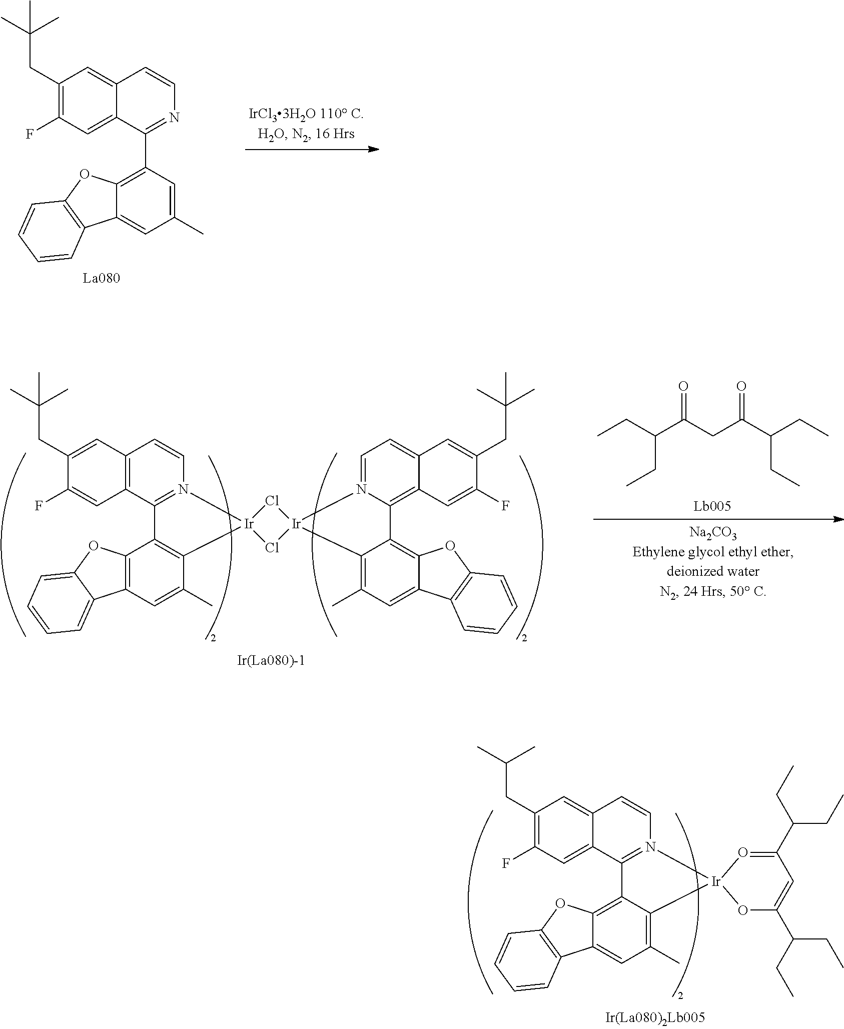

Synthesis of a Compound Ir(La080)-1:

With reference to the synthesis and purification methods of the compound Ir(La001)-1, only the corresponding raw materials were required to be changed, and a compound Ir(La080)-1 was obtained and directly used in the next step without purification.

Synthesis of a Compound Ir(La080)₂Lb005:

With reference to the synthesis and purification methods of the compound Ir(La001)₂Lb005, only the corresponding raw materials were required to be changed, and 1.87 g of a red solid, namely compound Ir(La080)₂Lb005, with a yield of 43.22% was obtained. 1.87 g of the crude product Ir(La080)₂Lb005 was sublimated and purified to obtain 1.04 g of sublimated pure Ir(La080)₂Lb005 with a yield of 55.61%. The mass spectrum was: 1197.49 (M+H). ¹H NMR (400 MHz, CDCl₃) δ 8.77 (d, J=8.6 Hz, 2H), 8.35 (d, J=6.6 Hz, 2H), 7.82 (d, J=7.4 Hz, 2H), 7.59 (d, J=6.5 Hz, 2H), 7.54-7.44 (m, 4H), 7.43-7.34 (m, 4H), 7.31 (d, J=7.3 Hz, 2H), 4.83 (s, 1H), 2.83 (s, 4H), 2.19-2.08 (m, 2H), 1.86 (s, 6H), 1.27 (m, 4H), 1.01 (m, 4H), 0.94 (s, 12H), 0.85 (s, 18H).

Synthesis of a Compound La106:

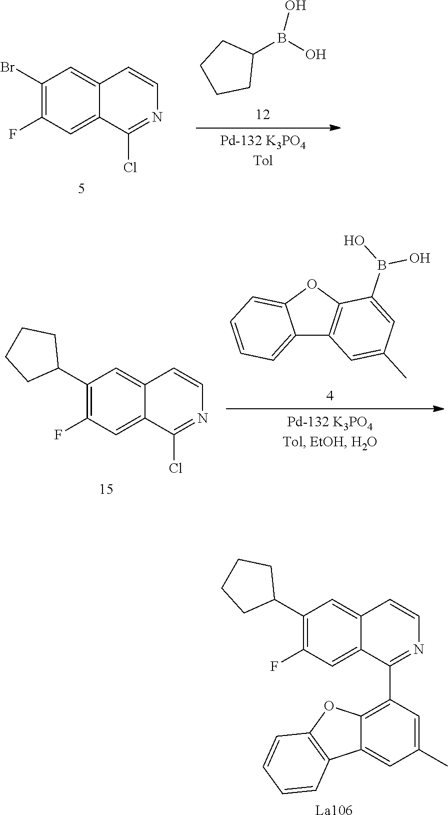

Synthesis of a Compound 15:

With reference to the synthesis and purification methods of the compound 3, only the corresponding raw materials were required to be changed, and a target compound 15 was obtained. The mass spectrum was 250.71 (M+H).

Synthesis of a Compound La106:

With reference to the synthesis and purification methods of the compound La001, only the corresponding raw materials were required to be changed, and a target compound La106 was obtained. The mass spectrum was 396.47 (M+H).

Synthesis of a Compound Ir(La106)₂Lb005:

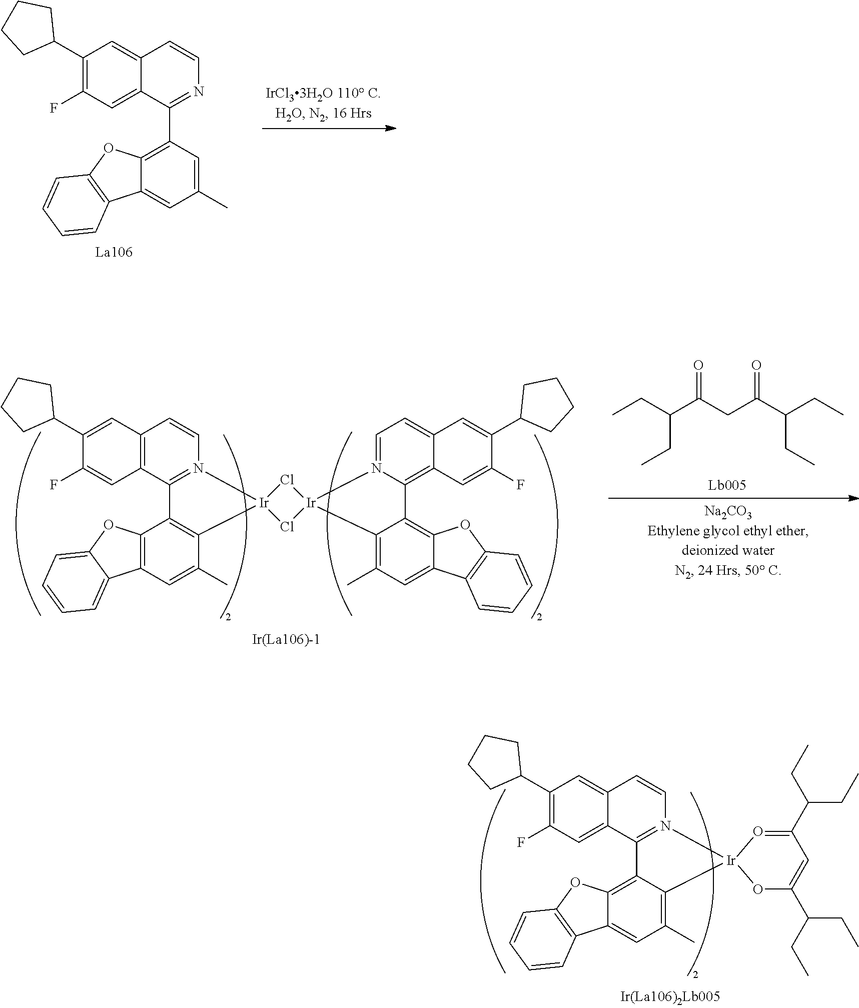

Synthesis of a Compound Ir(La106)-1:

With reference to the synthesis and purification methods of the compound Ir(La001)-1, only the corresponding raw materials were required to be changed, and a compound Ir(La106)-1 was obtained and directly used in the next step without purification.

Synthesis of a Compound Ir(La106)₂Lb005:

With reference to the synthesis and purification methods of the compound Ir(La001)₂Lb005, only the corresponding raw materials were required to be changed, and 2.06 g of a red solid, namely compound Ir(La106)₂Lb005, with a yield of 45.77% was obtained. 2.06 g of the crude product Ir(La106)₂Lb005 was sublimated and purified to obtain 1.28 g of sublimated pure Ir(La106)₂Lb005 with a yield of 62.13%. The mass spectrum was: 1193.46 (M+H). ¹H NMR (400 MHz, CDCl₃) δ 8.76 (d, J=8.6 Hz, 2H), 8.34 (d, J=6.6 Hz, 2H), 7.80 (d, J=7.4 Hz, 2H), 7.57 (d, J=6.5 Hz, 2H), 7.52-7.42 (m, 4H), 7.41-7.31 (m, 4H), 7.28 (d, J=7.3 Hz, 2H), 4.83 (s, 1H), 2.19-2.08 (m, 2H), 1.86 (s, 6H), 1.62 (m, 4H), 1.43 (m, 8H), 1.31 (m, 4H), 1.24 (m, 4H), 1.01 (m, 6H), 0.94 (s, 12H).

Synthesis of a Compound La171:

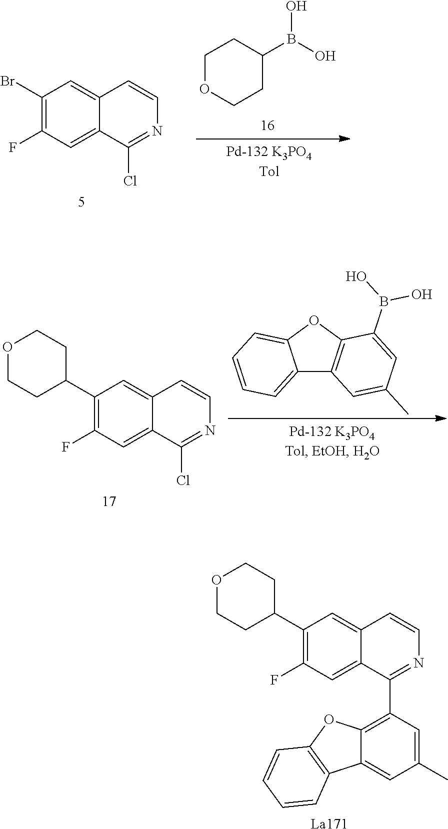

Synthesis of a Compound 17:

With reference to the synthesis and purification methods of the compound 3, only the corresponding raw materials were required to be changed, and a target compound 17 was obtained. The mass spectrum was 266.71 (M+H).

Synthesis of a Compound La171:

With reference to the synthesis and purification methods of the compound La001, only the corresponding raw materials were required to be changed, and a target compound La171 was obtained. The mass spectrum was 412.47 (M+H).

Synthesis of a Compound Ir(La171)₂Lb005:

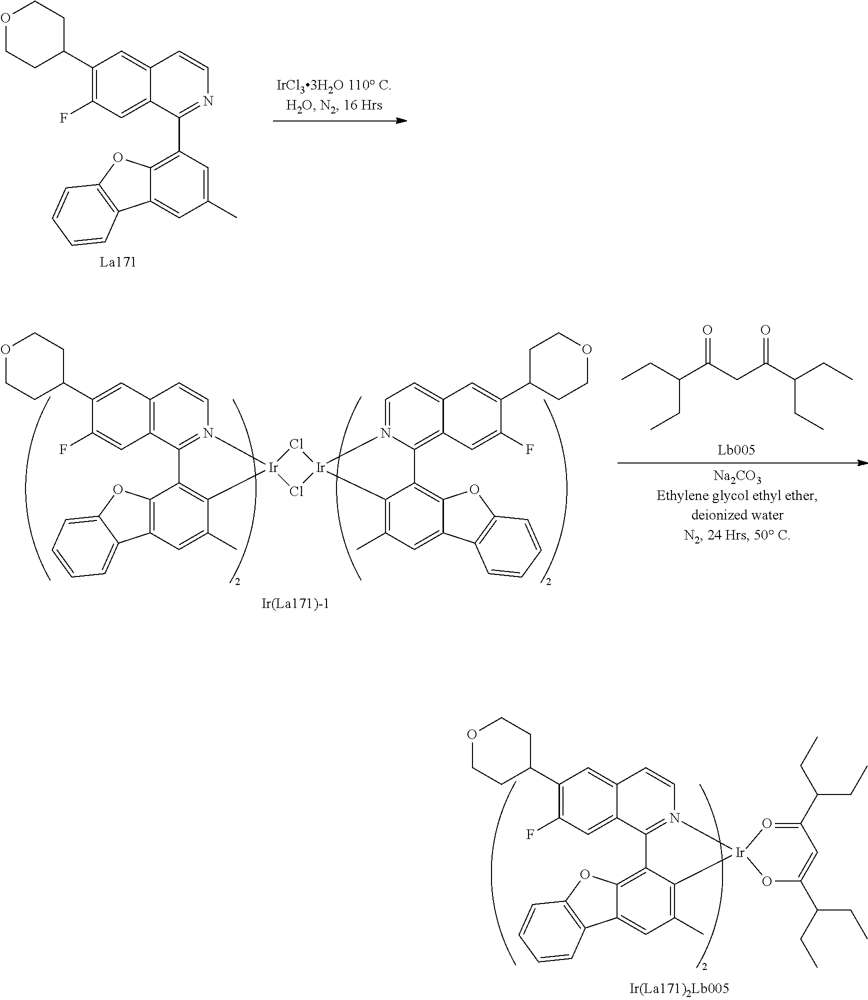

Synthesis of a Compound Ir(La171)-1:

With reference to the synthesis and purification methods of the compound Ir(La001)-1, only the corresponding raw materials were required to be changed, and a compound Ir(La171)-1 was obtained and directly used in the next step without purification.

Synthesis of a Compound Ir(La171)₂Lb005:

With reference to the synthesis and purification methods of the compound Ir(La001)₂Lb005, only the corresponding raw materials were required to be changed, and 1.82 g of a red solid, namely compound Ir(La171)₂Lb005, with a yield of 34.87% was obtained. 1.82 g of the crude product Ir(La171)₂Lb005 was sublimated and purified to obtain 1.01 g of sublimated pure Ir(La171)₂Lb005 with a yield of 55.49%. The mass spectrum was: 1225.46 (M+H). ¹H NMR (400 MHz, CDCl₃) δ 8.81 (d, J=8.6 Hz, 2H), 8.37 (d, J=6.6 Hz, 2H), 7.86 (d, J=7.4 Hz, 2H), 7.61 (d, J=6.5 Hz, 2H), 7.58-7.47 (m, 4H), 7.44-7.33 (m, 4H), 7.31 (d, J=7.3 Hz, 2H), 4.82 (s, 1H), 2.23-2.14 (m, 2H), 1.88 (s, 6H), 1.64 (m, 4H), 1.51 (m, 4H), 1.41 (m, 6H), 1.27 (m, 8H), 1.07-0.89 (m, 16H).

### Application Example: Manufacture of an Organic Electroluminescent Device

A glass substrate with a size of 50 mm*50 mm*1.0 mm including an ITO anode electrode (70 Å/1,000 Å/110 Å) was ultrasonically cleaned in ethanol for 10 minutes, dried at 150° C., and then treated with N₂ plasma for 30 minutes. The washed glass substrate was installed on a substrate support of a vacuum evaporation device. First, compounds HTM1 and P-dopant (at a ratio of 97%:3%) for covering the electrode were co-evaporated on the surface of the side having an anode electrode line to form a thin film having a thickness of 100 Å. Then, a layer of HTM1 was evaporated to form a thin film having a thickness of 1,720 Å. Then, a layer of HTM2 was evaporated on the HTM1 thin film to form a thin film having a thickness of 100 Å. After that, a main material 1, a main material 2 and a doping compound (including a reference compound X and the compound of the present invention) were co-evaporated on the HTM2 film layer at a ratio of 48.5%:48.5%:3% to form a film having a thickness of 400 Å, where the ratio of the main materials to the doping material was 90%:10%. ETL and LiQ were co-evaporated on a light-emitting layer at a ratio of 50%:50% to obtain reach a thickness of 350 Å. Then, Yb was evaporated on an electron transport layer to reach a thickness of 10 Å. Finally, a layer of metal Ag was evaporated to serve as an electrode having a thickness of 150 Å.

| | HIL | HTL | EBL | | Electron |
|---|---|---|---|---|---|
| | Thickness/ | Thickness/ | Thickness/ | Emission layer | transport layer |
| Example | Å | Å | Å | Thickness/Å | Thickness/Å |
| A1 | HTM1: | HTM1 | HTM2 | H1: H2: | ETL: LiQ |
| | NDP-9 | 1720 | 100 | Ir(La001)₂Lb005 | 350 |
| | 100 | | | 400 | |
| A2 | HTM1: | HTM1 | HTM2 | H1: H2: | ETL: LiQ |
| | NDP-9 | 1720 | 100 | Ir(La002)₂Lb005 | 350 |
| | 100 | | | 400 | |
| A3 | HTM1: | HTM1 | HTM2 | H1: H2: | ETL: LiQ |
| | NDP-9 | 1720 | 100 | Ir(La027)₂Lb005 | 350 |
| | 100 | | | 400 | |
| A4 | HTM1: | HTM1 | HTM2 | H1: H2: | ETL: LiQ |
| | NDP-9 | 1720 | 100 | Ir(La027)₂Lb031 | 350 |
| | 100 | | | 400 | |
| A5 | HTM1: | HTM1 | HTM2 | H1: H2 | ETL: LiQ |
| | NDP-9 | 1720 | 100 | Ir(La028)₂Lb005 | 350 |
| | 100 | | | 400 | |
| A6 | HTM1: | HTM1 | HTM2 | H1: H2: | ETL: LiQ |
| | NDP-9 | 1720 | 100 | Ir(La037)₂Lb005 | 350 |
| | 100 | | | 400 | |
| A7 | HTM1: | HTM1 | HTM2 | H1: H2 | ETL: LiQ |
| | NDP-9 | 1720 | 100 | Ir(La080)₂Lb005 | 350 |
| | 100 | | | 400 | |
| A8 | HTM1: | HTM1 | HTM2 | H1: H2: | ETL: LiQ |
| | NDP-9 | 1720 | 100 | Ir(La106)₂Lb005 | 350 |
| | 100 | | | 400 | |
| A9 | HTM1: | HTM1 | HTM2 | H1: H2: | ETL: LiQ |
| | NDP-9 | 1720 | 100 | Ir(La171)₂Lb005 | 350 |
| | 100 | | | 400 | |
| Comparative | HTM1: | HTM1 | HTM2 | H1: H2: reference | ETL: LiQ |
| Example 1 | NDP-9 | 1720 | 100 | compound 1 | |
| | 100 | | | 400 | |
| Comparative | HTM1: | HTM1 | HTM2 | H1: H2: reference | 350 |
| Example 2 | NDP-9 | 1720 | 100 | compound 2 | |
| | 100 | | | 400 | |
| Comparative | HTM1: | HTM1 | HTM2 | H1: H2: reference | ETL: LiQ |
| Example 3 | NDP-9 | 1720 | 100 | compound 3 | |
| | 100 | | | 400 | |
| Comparative | HTM1: | HTM1 | HTM2 | H1: H2: reference | 350 |
| Example 4 | NDP-9 | 1720 | 100 | compound 4 | |
| | 100 | | | 400 | |
| Comparative | HTM1: | HTM1 | HTM2 | H1: H2: reference | ETL: LiQ |
| Example 5 | NDP-9 | 1720 | 100 | compound 5 | |
| | 100 | | | 400 | |
| Comparative | HTM1: | HTM1 | HTM2 | H1: H2: reference | ETL: LiQ |
| Example 6 | NDP-9 | 1720 | 100 | compound 6 | |
| | 100 | | | 400 | |

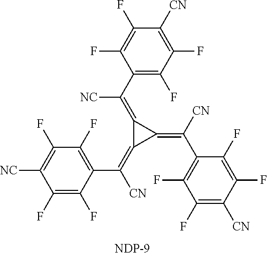

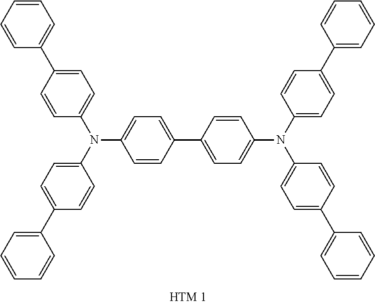 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

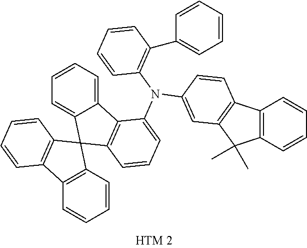 [SMILES: CC1(C)c2ccccc2-c2ccc(N(c3ccccc3-c3ccccc3)c3cccc4c3-c3ccccc3C43c4ccccc4-c4ccccc43)cc21]

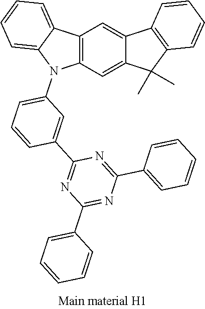 [SMILES: CC1(C)c2ccccc2-c2cc3c4ccccc4n(-c4cccc(-c5nc(-c6ccccc6)nc(-c6ccccc6)n5)c4)c3cc21]

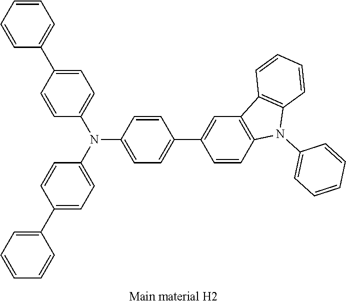 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

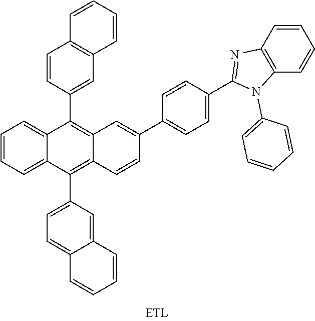 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

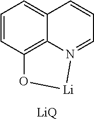

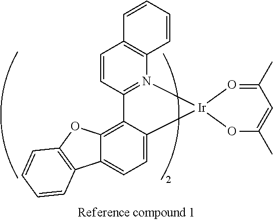

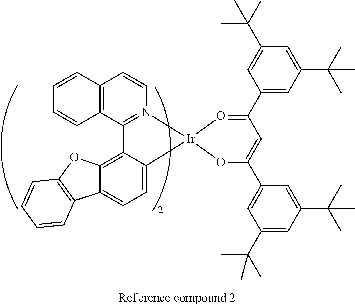

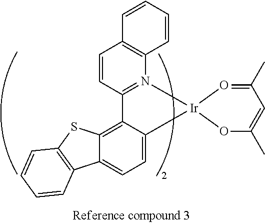

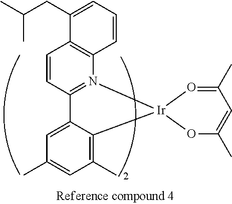

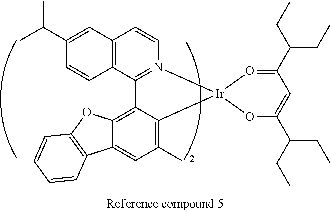

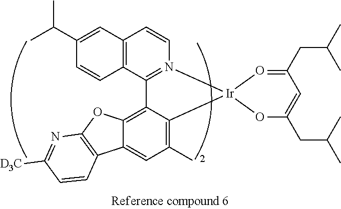

Evaluation: Properties of a device obtained above were tested. In various examples and comparative examples, a constant-current power supply (Keithley 2400) was used, a current at a fixed density was used for flowing through light-emitting elements, and a spectroradiometer (CS 2000) was used for testing the light-emitting spectrum. Meanwhile, the voltage value was measured, and the time (LT90) when the brightness was reduced to 90% of the initial brightness was tested. Results are as follows. The current efficiency and the device service life are calculated with the value of the reference compound 5 as 100%.

| | Starting | Current | Chromaticity | |
|---|---|---|---|---|
| | voltage@20 | efficiency | coordinate | |
| | mA/cm² | @20 mA/ | @20 mA/cm2 | LT90@ |
| | V | cm2 | CIEx, CIEy | 8000nits |
| Example A1 | 4.41 | 130 | 0.700, 0.298 | 152 |
| Example A2 | 4.42 | 133 | 0.701, 0.298 | 139 |
| Example A3 | 4.36 | 138 | 0.701, 0.298 | 148 |
| Example A4 | 4.37 | 142 | 0.702, 0.297 | 172 |
| Example A5 | 4.38 | 136 | 0.702, 0.298 | 139 |
| Example A6 | 4.32 | 142 | 0.702, 0.296 | 168 |
| Example A7 | 4.34 | 139 | 0.700, 0.299 | 136 |
| Example A8 | 4.35 | 140 | 0.701, 0.298 | 147 |
| Example A9 | 4.36 | 141 | 0.702, 0.297 | 144 |
| Comparative | 5.23 | 75 | 0.700, 0.299 | 51 |
| Example 1 | | | | |
| Comparative | 5.15 | 72 | 0.701, 0.298 | 50 |
| Example 2 | | | | |
| Comparative | 5.34 | 74 | 0.703, 0.296 | 42 |
| Example 3 | | | | |
| Comparative | 5.52 | 63 | 0.702, 0.297 | 37 |
| Example 4 | | | | |
| Comparative | 4.88 | 100 | 0.701, 0.298 | 100 |
| Example 5 | | | | |
| Comparative | 4.74 | 95 | 0.702, 0.298 | 118 |
| Example 6 | | | | |

Through comparison of the data in the above table, it can be seen that compared with reference compounds, the compound of the present invention used as a dopant in organic electroluminescent devices with the same chromaticity coordinate has more excellent properties, such as driving voltage, luminous efficiency, and device service life.

The comparison of emission wavelengths in a dichloromethane solution is defined as follows. A corresponding compound is prepared into a 10⁻⁵ mol/L solution with dichloromethane, and the emission wavelength is tested by Hitachi (HITACH) F2700 fluorescence spectrophotometer to obtain the wavelength at a maximum emission peak. Test results are shown as follows.

| Material | PL peak wavelength/nm |
|---|---|
| Ir(La001)₂ Lb005 | 627 |
| Ir(La002)₂ Lb005 | 629 |
| Ir(La027)₂ Lb005 | 626 |
| Ir(La027)₂ Lb031 | 627 |
| Ir(La028)₂ Lb005 | 626 |
| Ir(La037)₂ Lb005 | 630 |
| Ir(La080)₂ Lb005 | 627 |
| Ir(La106)₂ Lb005 | 628 |
| Ir(La171)₂ Lb005 | 629 |
| Reference compound 1 | 610 |
| Reference compound 2 | 637 |
| Reference compound 3 | 611 |
| Reference compound 4 | 608 |
| Reference compound 5 | 616 |

Through comparison of the data in the above table, it can be seen that compared with reference compounds, the metal iridium complex of the present invention has a larger red shift, so that industrial demands for dark red light, especially the BT2020 color gamut, can be met.

Comparison of the sublimation temperature is as follows. The sublimation temperature is defined as the temperature corresponding to an evaporation rate of 1 Å/s at a vacuum degree of 10⁻⁷ Torr. Test results are shown as follows.

| Material | Sublimation temperature |
|---|---|
| Ir(La001)₂ Lb005 | 255 |
| Ir(La002)₂ Lb005 | 257 |
| Ir(La027)₂ Lb005 | 260 |
| Ir(La027)₂ Lb031 | 262 |
| Ir(La028)₂ Lb005 | 263 |
| Ir(La037)₂ Lb005 | 259 |
| Ir(La080)₂ Lb005 | 263 |
| Ir(La106)₂ Lb005 | 264 |
| Ir(La171)₂ Lb005 | 265 |
| Reference compound 1 | 280 |
| Reference compound 2 | 288 |
| Reference compound 3 | 286 |
| Reference compound 4 | 276 |
| Reference compound 5 | 268 |

Through comparison of the data in the above table, it can be seen that the metal iridium complex of the present invention has low sublimation temperature, and industrial application is facilitated.

Compared with the prior art, the present invention unexpectedly provides better device luminous efficiency, improved service life, lower sublimation temperature and more saturated red luminescence through special collocation of substituents. According to the above results, it is indicated that the compound of the present invention has the advantages of low sublimation temperature, high optical and electrochemical stability, high color saturation, high luminous efficiency, and long device service life, and can be used in organic electroluminescent devices. In particular, the metal complex has the potential for application in the OLED industry as a red light-emitting dopant, especially in displays, lighting and car tail lights.

The compound of the present invention has the advantages of high optical and electrochemical stability, high color saturation, high luminous efficiency, and long device service life, and can be used in organic electroluminescent devices. In particular, the metal complex has the potential for application in the OLED industry as a red light-emitting dopant.

1. A metal complex, having a general formula of Ir(La)(Lb)(Lc) and a structure as shown in a formula (1): 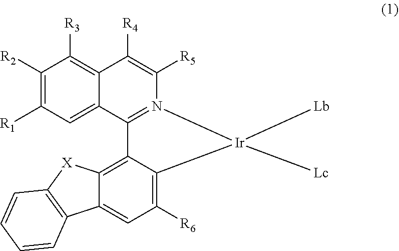
wherein 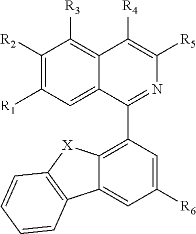
is a ligand La;
X is independently selected from O, S, and Se;
R₁-R₅ are independently selected from hydrogen, deuterium, halogen, substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, substituted or unsubstituted heterocycloalkyl containing 3-20 ring forming carbon atoms, substituted or unsubstituted C₃-C₃₀ alkylsilyl, substituted or unsubstituted C₁-C₁₀ alkoxyl, substituted or unsubstituted C₇-C₃₀ aralkyl, substituted or unsubstituted C₆-C₃₀ aryloxyl, substituted or unsubstituted C₂-C₂₀ alkenyl, substituted or unsubstituted C₂-C₂₀ alkynyl, substituted or unsubstituted C₆-C₃₀ aryl, substituted or unsubstituted C₃-C₃₀ heteroaryl, substituted or unsubstituted C₃-C₃₀ arylsilyl, substituted or unsubstituted C₀-C₂₀ alkylamino, cyano, iso cyano, and phosphino;
at least one of the R₁-R₅ is F, and one of the R₁-R₅ is substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, or substituted or unsubstituted heterocycloalkyl containing 3-20 ring forming carbon atoms;
R₆ is substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, or substituted or unsubstituted heterocycloalkyl containing 3-20 ring forming carbon atoms;
the “substituted” refers to substitution with deuterium, F, Cl, Br, C₁-C₄ alkyl, C₁-C₄ alkoxyl, C₃-C₆ cycloalkyl, amino substituted with C₁-C₄ alkyl, cyano, isocyano, or phosphino;
the heteroalkyl, the heterocycloalkyl, or the heteroaryl comprises at least one of S, O, and N heteroatom s;
Lb and Lc are both a monoanionic bidentate ligand, any two of the La, the Lb, and the Lc are connected to each other to form a multidentate ligand, or the La, the Lb, and the Lc are connected by a group;
and at least two of the La, the Lb, and the Lc are the same.

2. The metal complex according to claim 1, wherein the Lb has a structure as shown in a formula (2): 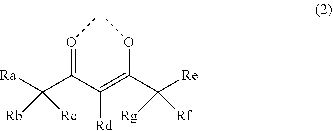
wherein a dotted line refers to a position connected to metal Ir;
Rₐ-R_{g} are independently selected from hydrogen, deuterium, halogen, substituted or unsubstituted alkyl containing 1-10 carbon atoms on a main chain, substituted or unsubstituted cycloalkyl containing 3-20 ring forming carbon atoms, substituted or unsubstituted heteroalkyl containing 1-10 carbon atoms on a main chain, and substituted or unsubstituted heterocycloalkyl containing 3-20 ring forming carbon atoms, or any two of Rₐ, R_{b}, and R_{c} are connected to each other to form an aliphatic ring structure, and any two of Rₑ, R_{f}, and R_{g} are connected to each other to form an aliphatic ring structure; and the “substituted” refers to substitution with deuterium, F, Cl, Br, C₁-C₄ alkyl, C₁-C₄ alkoxyl, C₃-C₆ cycloalkyl, amino substituted with C₁-C₄ alkyl, cyano, iso cyano, or phosphino.

3. The metal complex according to claim 2, wherein the Lc and the La have the same structure, so that a (La)₂Ir(Lb) structure is formed.

4. The metal complex according to claim 3, wherein the Rₐ, the R_{b}, and the R_{c} are the same as the Rₑ, the R_{f}, and the R_{g}, respectively.

5. The metal complex according to claim 4, wherein the Rₐ, the R_{b}, the R_{c}, the Rₑ, the R_{f}, and the R_{g} are independently selected from hydrogen, deuterium, halogen, substituted or unsubstituted alkyl containing 1-10 carbon atoms on a main chain, and substituted or unsubstituted cycloalkyl containing 3-20 ring forming carbon atoms, or any two of the Rₐ, the R_{b}, and the R_{c} are connected to each other to form an aliphatic ring structure, and any two of the Rₑ, the R_{f}, and the R_{g} are connected to each other to form an aliphatic ring structure; the “substituted” refers to substitution with deuterium, F, Cl, Br, C₁-C₄ alkyl, or C₃-C₆ cycloalkyl; and R_{d} is selected from hydrogen, deuterium, halogen, and substituted or unsubstituted alkyl containing 1-10 carbon atoms on a main chain.

6. The metal complex according to claim 3, wherein the Lb is independently selected from one of the following structural formulas, or corresponding parts or complete deuterides or fluorides thereof:

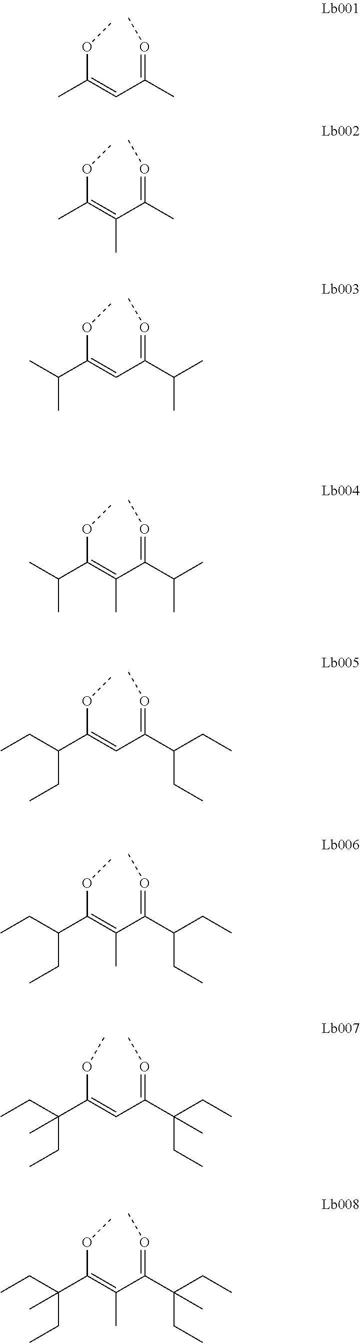

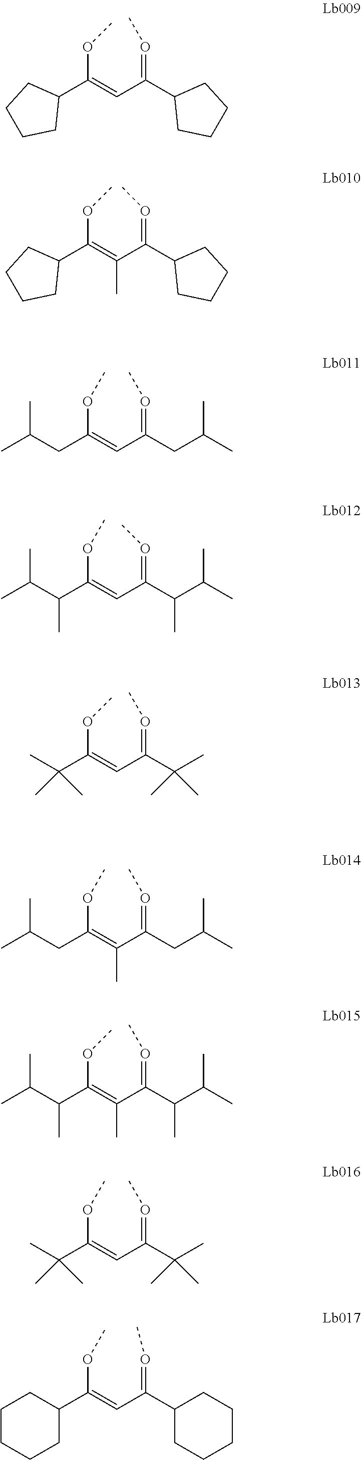

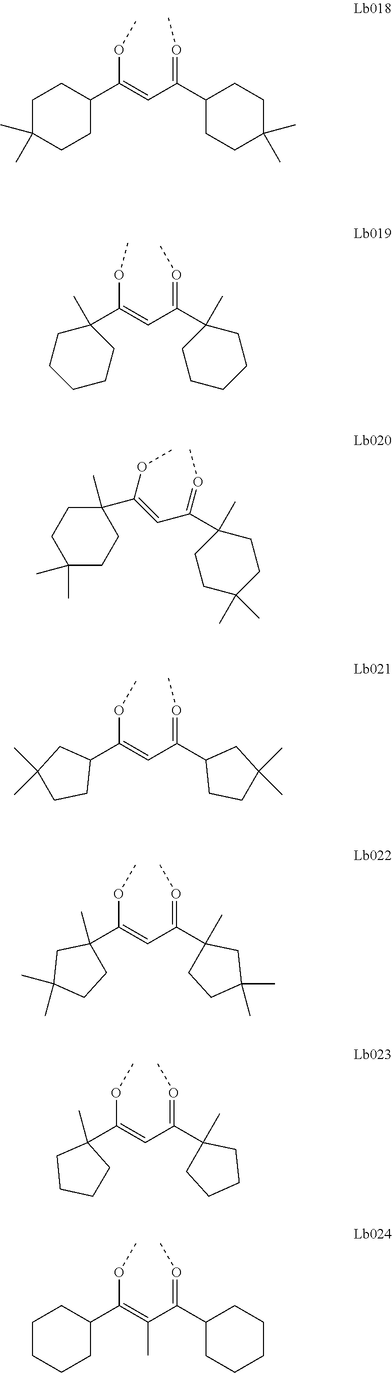

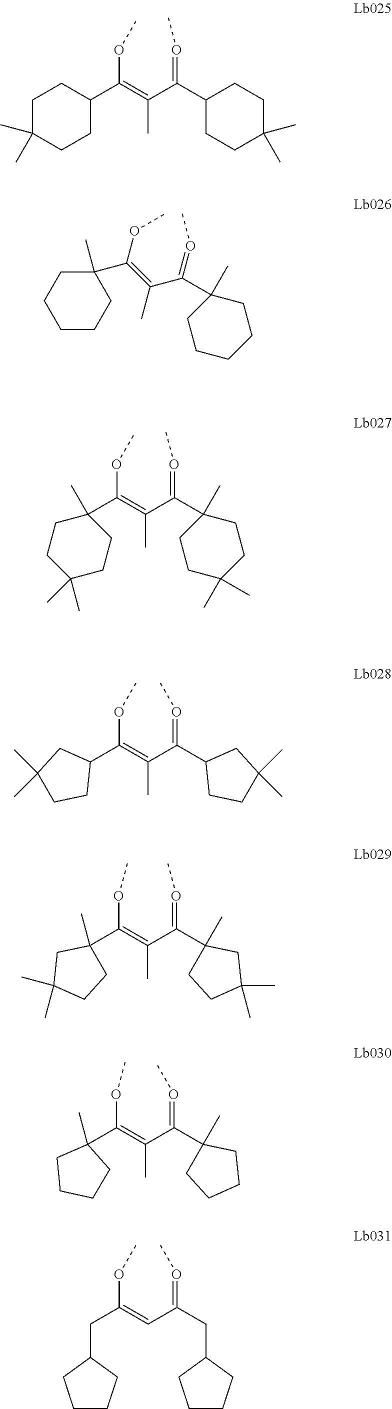

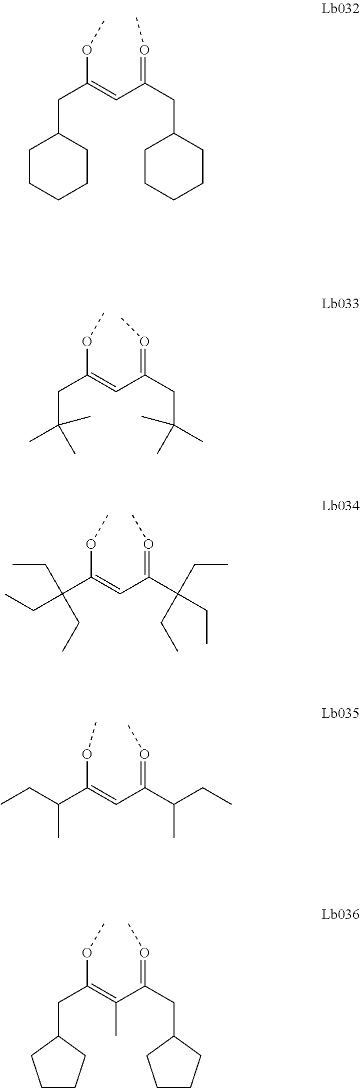

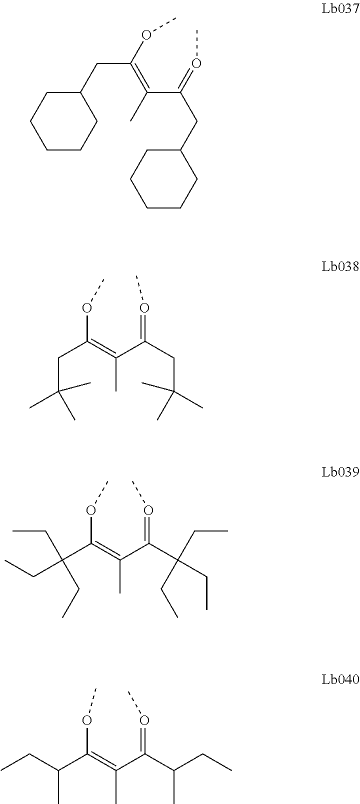

7. The metal complex according to claim 1, wherein the R₆ is substituted or unsubstituted alkyl containing no more than 4 carbon atoms on a main chain, or substituted or unsubstituted cycloalkyl containing no more than 6 ring forming carbon atoms.

8. The metal complex according to claim 7, wherein the F is not positioned at the R₅, and the X is an O atom.

9. The metal complex according to claim 8, wherein one of the R₁-R₅ is F, another one is substituted or unsubstituted alkyl containing no more than 4 carbon atoms on a main chain, or substituted or unsubstituted cycloalkyl containing no more than 6 ring forming carbon atoms, and the other three are hydrogen.

10. The metal complex according to claim 9, wherein when one of the R₁-R₅ is F, another one is branched alkyl substituted with C₁-C₄ alkyl containing no more than 4 carbon atoms on a main chain, and the other three are hydrogen.

11. The metal complex according to claim 1, wherein the La is independently selected from one of the following structural formulas, or corresponding parts or complete deuterides or fluorides thereof:

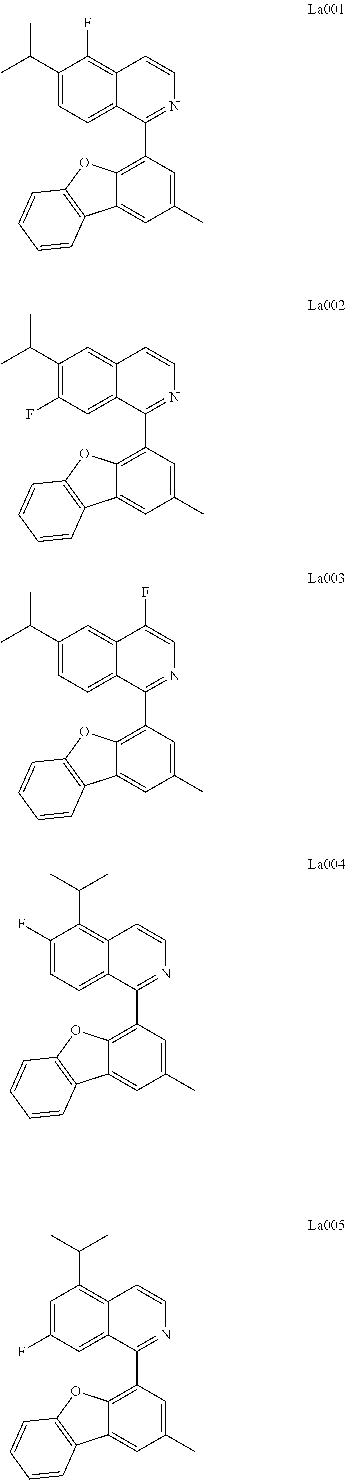

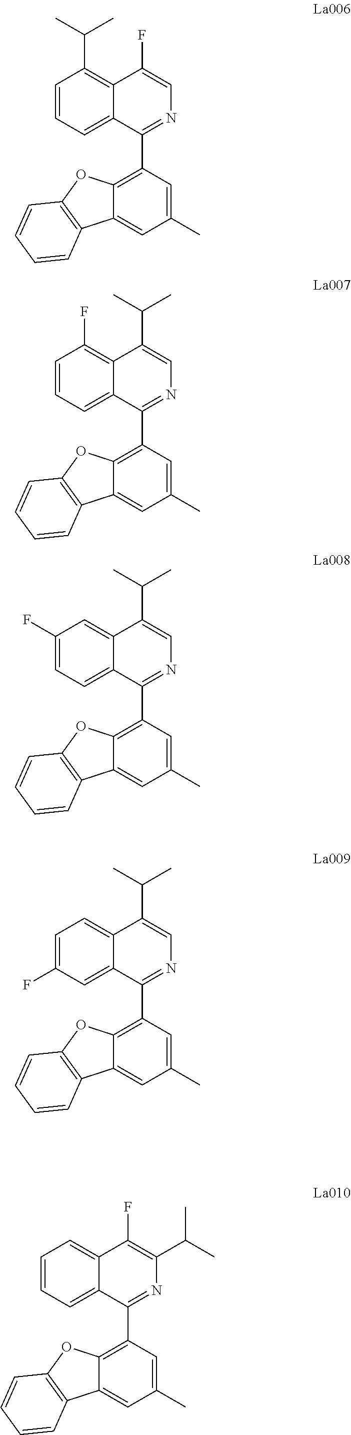 [SMILES: Cc1cc(-c2ncc(F)c3c(C(C)C)cccc23)c2oc3ccccc3c2c1 ; Cc1cc(-c2ncc(C(C)C)c3c(F)cccc23)c2oc3ccccc3c2c1 ; Cc1cc(-c2ncc(C(C)C)c3cc(F)ccc23)c2oc3ccccc3c2c1 ; Cc1cc(-c2ncc(C(C)C)c3ccc(F)cc23)c2oc3ccccc3c2c1 ; Cc1cc(-c2nc(C(C)C)c(F)c3ccccc23)c2oc3ccccc3c2c1]

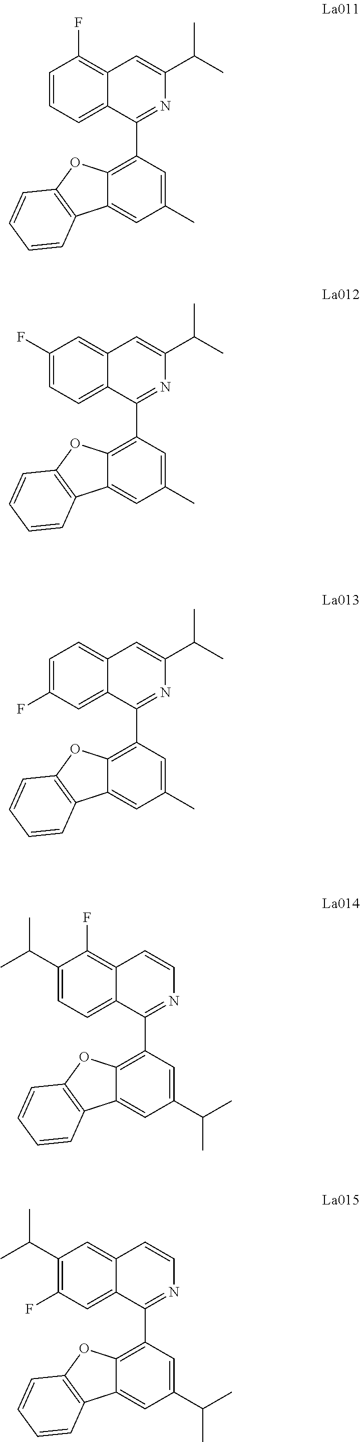

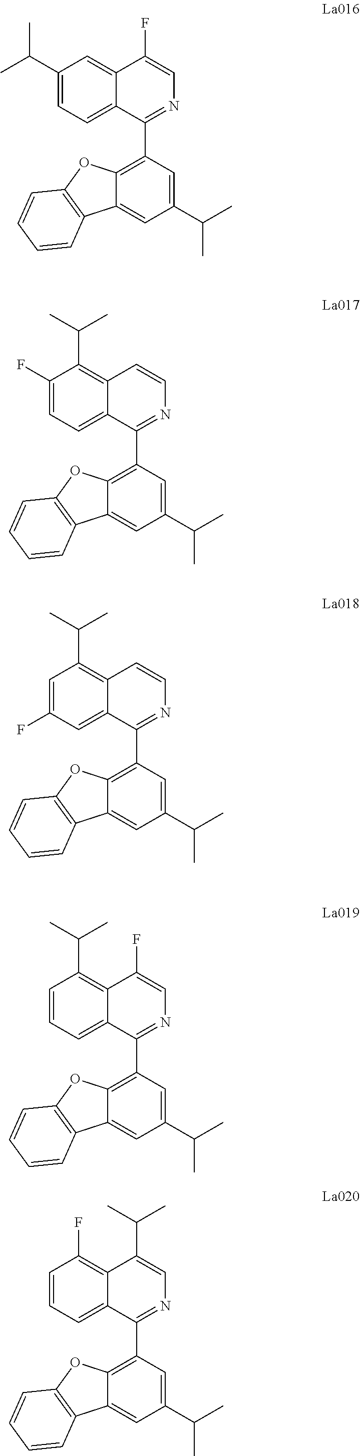

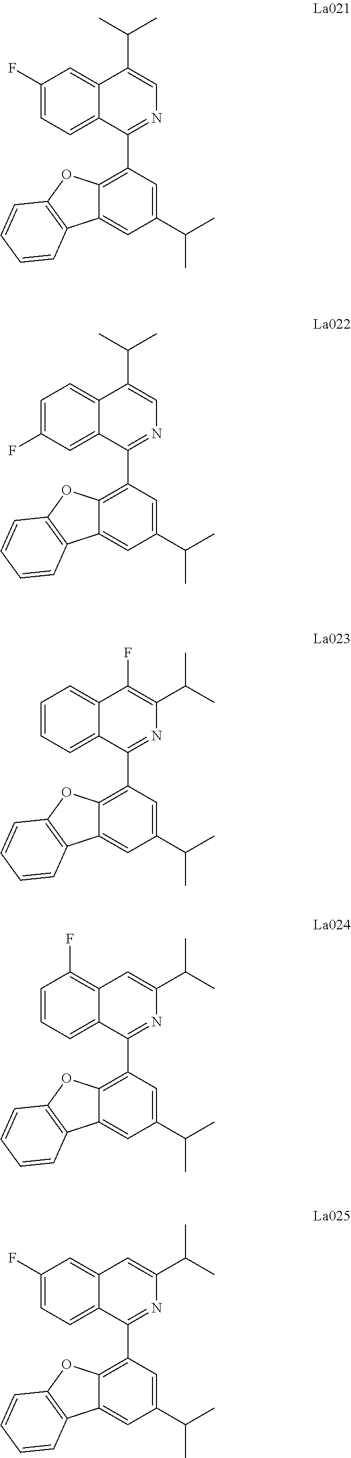 [SMILES: CC(C)c1cc(-c2ncc(C(C)C)c3cc(F)ccc23)c2oc3ccccc3c2c1 ; CC(C)c1cc(-c2ncc(C(C)C)c3ccc(F)cc23)c2oc3ccccc3c2c1 ; CC(C)c1cc(-c2nc(C(C)C)c(F)c3ccccc23)c2oc3ccccc3c2c1 ; CC(C)c1cc(-c2nc(C(C)C)cc3c(F)cccc23)c2oc3ccccc3c2c1 ; CC(C)c1cc(-c2nc(C(C)C)cc3cc(F)ccc23)c2oc3ccccc3c2c1]

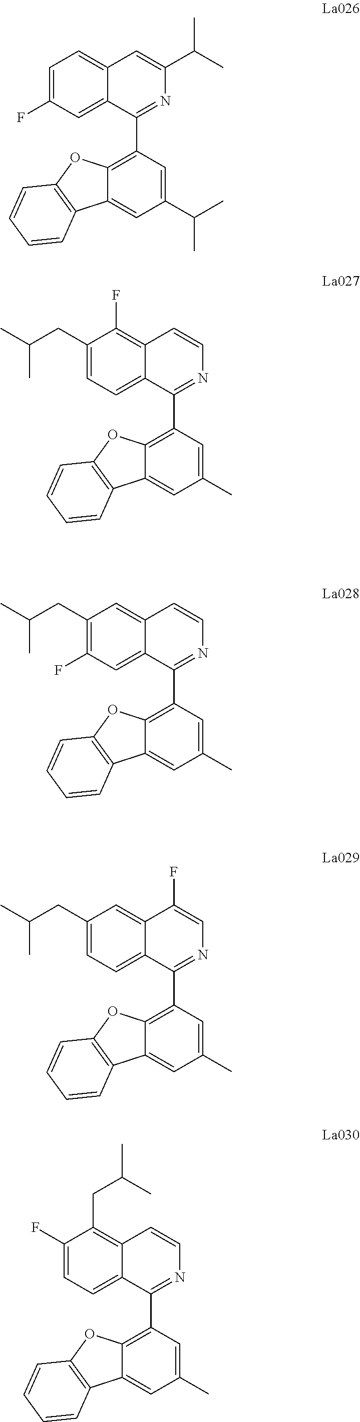

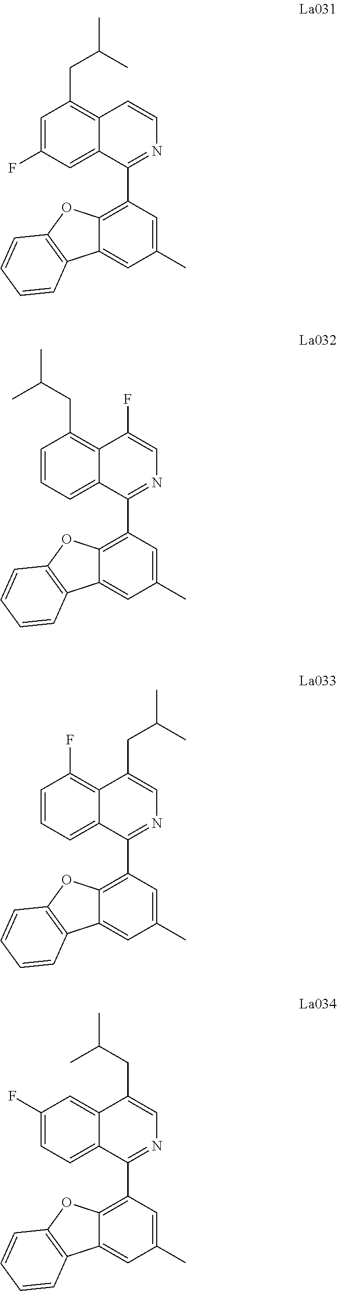 [SMILES: Cc1cc(-c2nccc3c(CC(C)C)cc(F)cc23)c2oc3ccccc3c2c1 ; Cc1cc(-c2ncc(F)c3c(CC(C)C)cccc23)c2oc3ccccc3c2c1 ; Cc1cc(-c2ncc(CC(C)C)c3c(F)cccc23)c2oc3ccccc3c2c1 ; Cc1cc(-c2ncc(CC(C)C)c3cc(F)ccc23)c2oc3ccccc3c2c1]

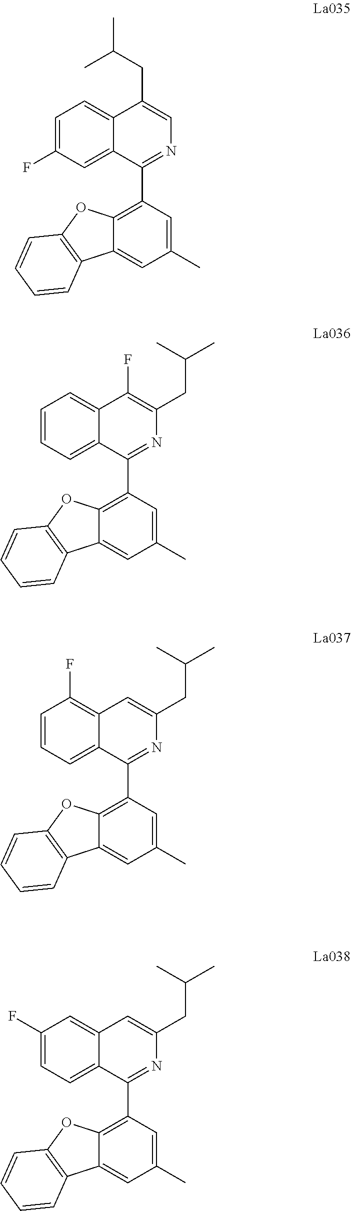 [SMILES: Cc1cc(-c2ncc(CC(C)C)c3ccc(F)cc23)c2oc3ccccc3c2c1 ; Cc1cc(-c2nc(CC(C)C)c(F)c3ccccc23)c2oc3ccccc3c2c1 ; Cc1cc(-c2nc(CC(C)C)cc3c(F)cccc23)c2oc3ccccc3c2c1 ; Cc1cc(-c2nc(CC(C)C)cc3cc(F)ccc23)c2oc3ccccc3c2c1]

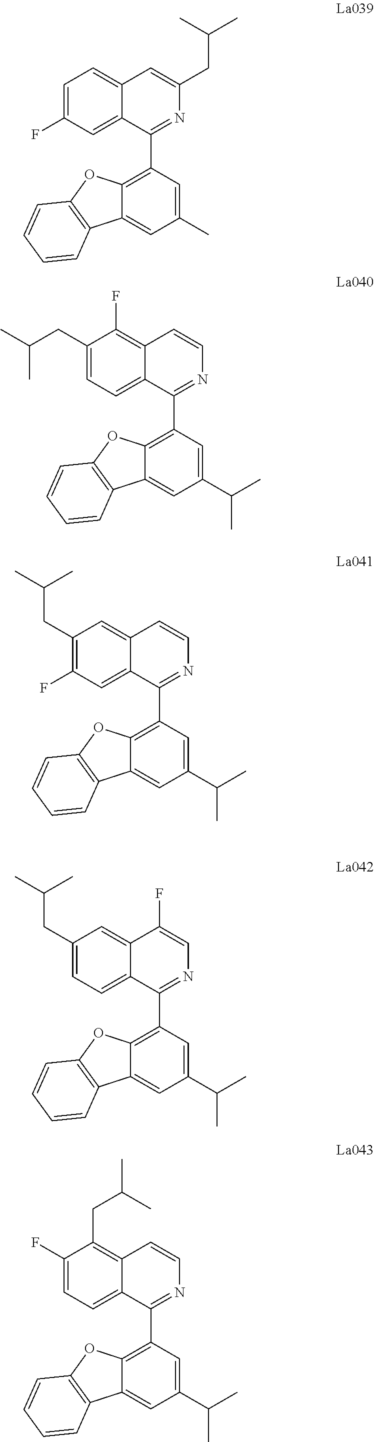

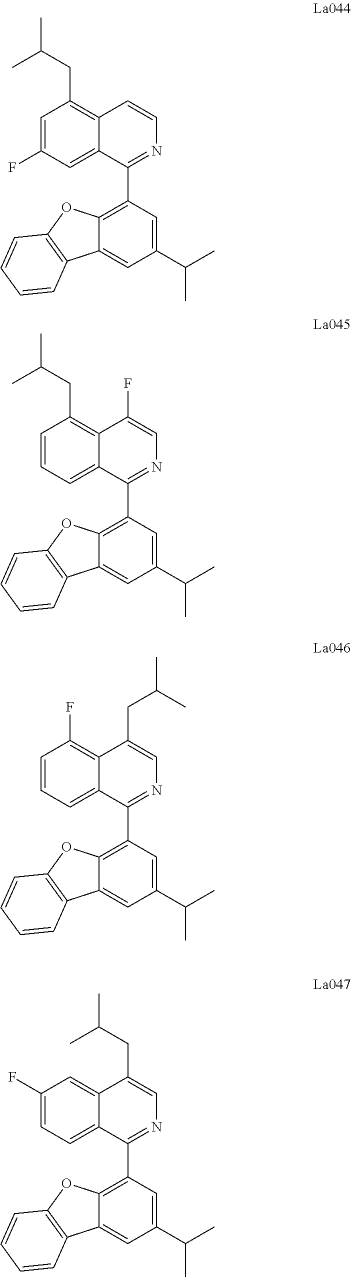

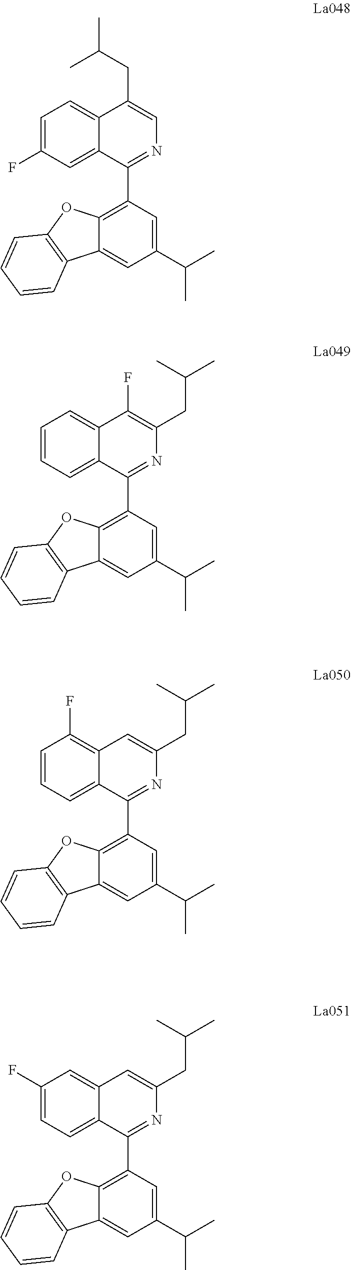 [SMILES: CC(C)Cc1cnc(-c2cc(C(C)C)cc3c2oc2ccccc23)c2cc(F)ccc12 ; CC(C)Cc1nc(-c2cc(C(C)C)cc3c2oc2ccccc23)c2ccccc2c1F ; CC(C)Cc1cc2c(F)cccc2c(-c2cc(C(C)C)cc3c2oc2ccccc23)n1 ; CC(C)Cc1cc2cc(F)ccc2c(-c2cc(C(C)C)cc3c2oc2ccccc23)n1]

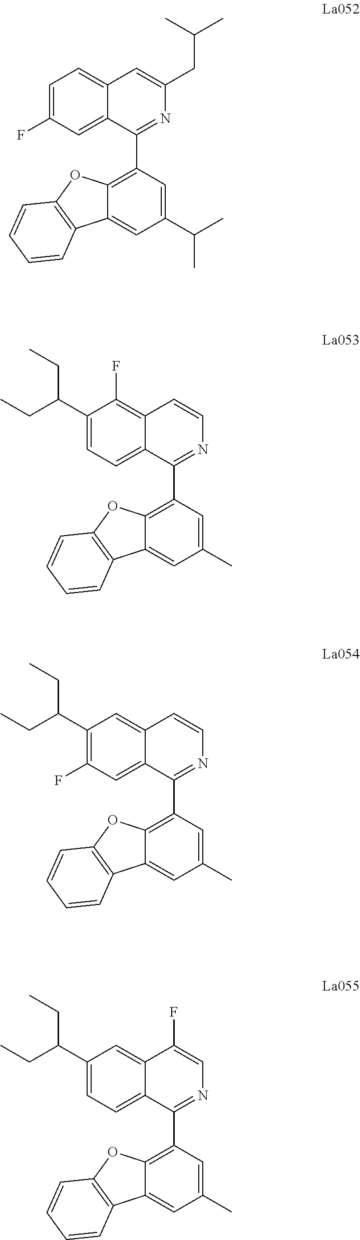

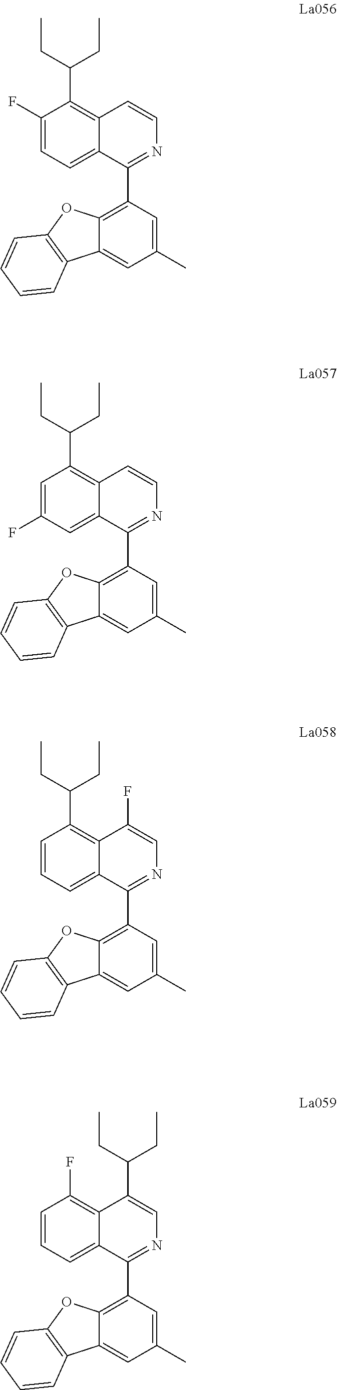 [SMILES: CCC(CC)c1c(F)ccc2c(-c3cc(C)cc4c3oc3ccccc34)nccc12 ; CCC(CC)c1cc(F)cc2c(-c3cc(C)cc4c3oc3ccccc34)nccc12 ; CCC(CC)c1cccc2c(-c3cc(C)cc4c3oc3ccccc34)ncc(F)c12 ; CCC(CC)c1cnc(-c2cc(C)cc3c2oc2ccccc23)c2cccc(F)c12]

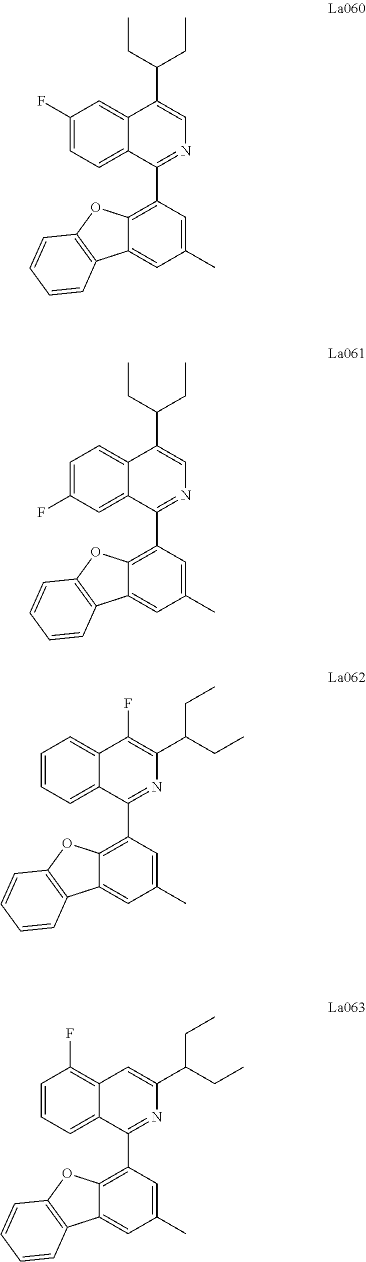 [SMILES: CCC(CC)c1cnc(-c2cc(C)cc3c2oc2ccccc23)c2ccc(F)cc12 ; CCC(CC)c1cnc(-c2cc(C)cc3c2oc2ccccc23)c2cc(F)ccc12 ; CCC(CC)c1nc(-c2cc(C)cc3c2oc2ccccc23)c2ccccc2c1F ; CCC(CC)c1cc2c(F)cccc2c(-c2cc(C)cc3c2oc2ccccc23)n1]

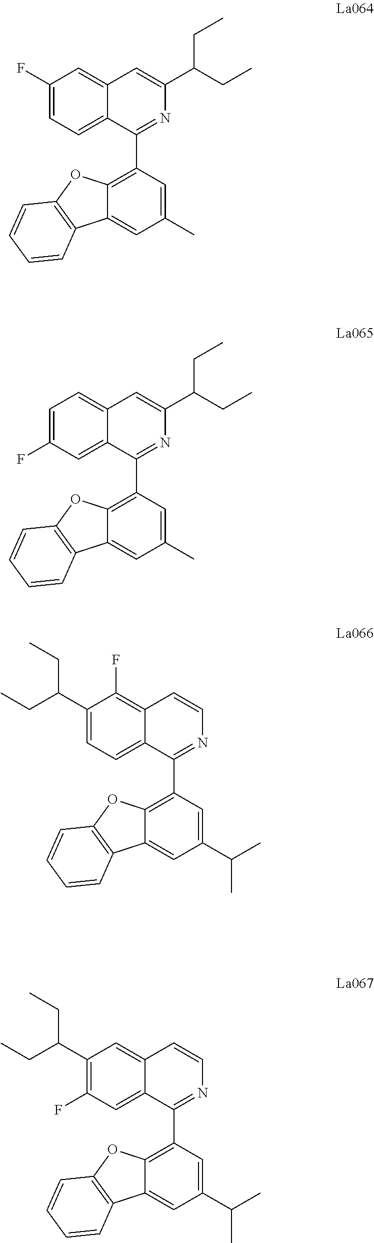

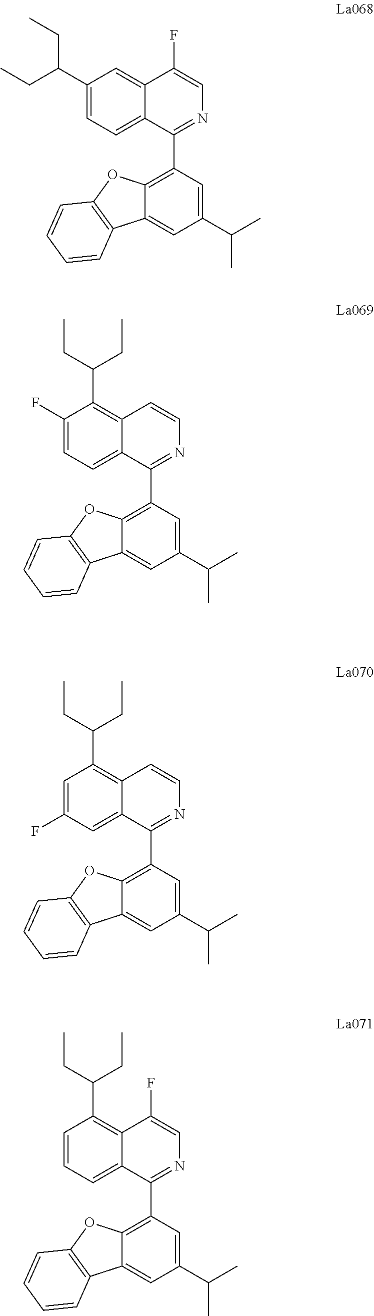 [SMILES: CCC(CC)c1ccc2c(-c3cc(C(C)C)cc4c3oc3ccccc34)ncc(F)c2c1 ; CCC(CC)c1c(F)ccc2c(-c3cc(C(C)C)cc4c3oc3ccccc34)nccc12 ; CCC(CC)c1cc(F)cc2c(-c3cc(C(C)C)cc4c3oc3ccccc34)nccc12 ; CCC(CC)c1cccc2c(-c3cc(C(C)C)cc4c3oc3ccccc34)ncc(F)c12]

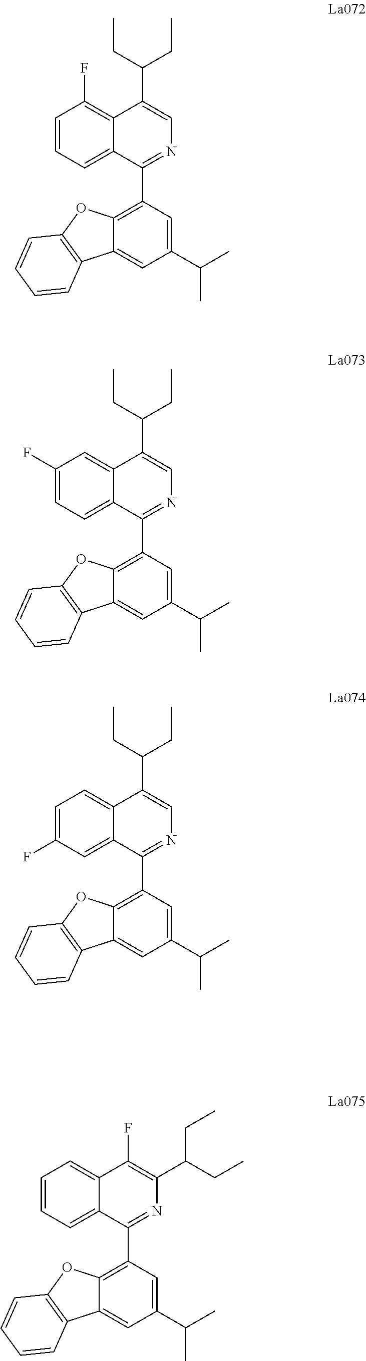 [SMILES: CCC(CC)c1cnc(-c2cc(C(C)C)cc3c2oc2ccccc23)c2cccc(F)c12 ; CCC(CC)c1cnc(-c2cc(C(C)C)cc3c2oc2ccccc23)c2ccc(F)cc12 ; CCC(CC)c1cnc(-c2cc(C(C)C)cc3c2oc2ccccc23)c2cc(F)ccc12 ; CCC(CC)c1nc(-c2cc(C(C)C)cc3c2oc2ccccc23)c2ccccc2c1F]

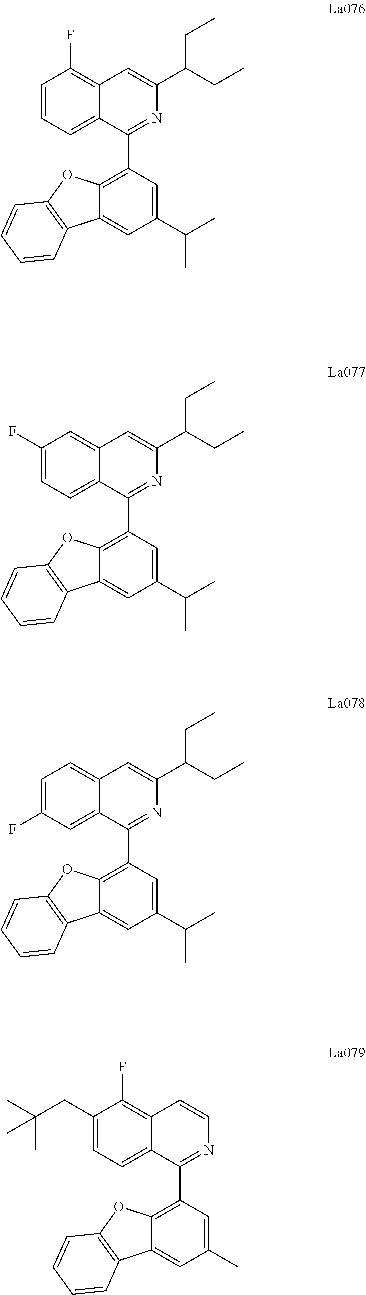

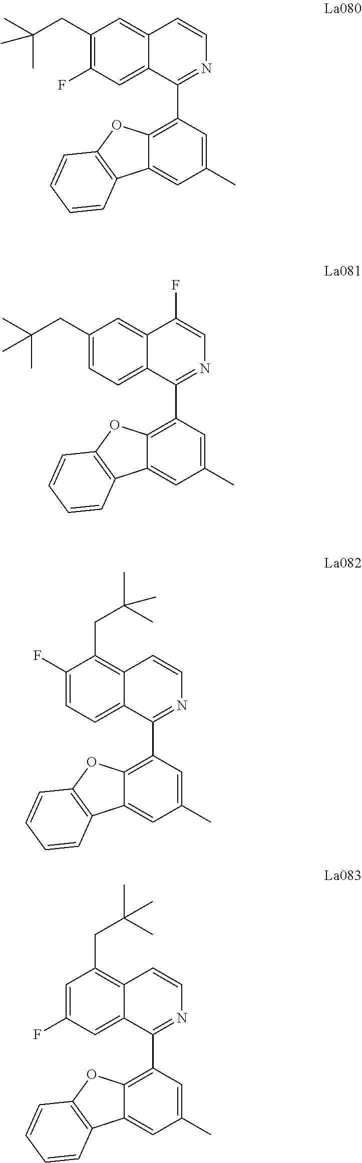

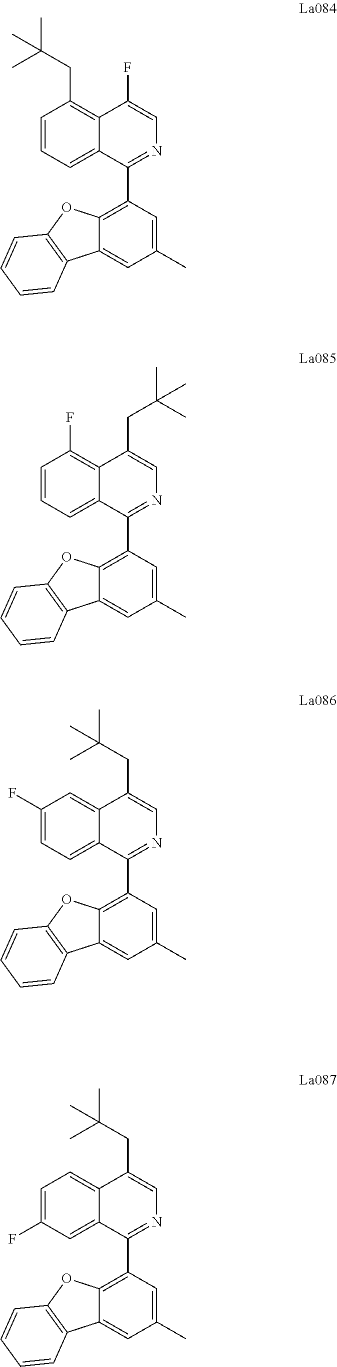 [SMILES: Cc1cc(-c2ncc(F)c3c(CC(C)(C)C)cccc23)c2oc3ccccc3c2c1 ; Cc1cc(-c2ncc(CC(C)(C)C)c3c(F)cccc23)c2oc3ccccc3c2c1 ; Cc1cc(-c2ncc(CC(C)(C)C)c3cc(F)ccc23)c2oc3ccccc3c2c1 ; Cc1cc(-c2ncc(CC(C)(C)C)c3ccc(F)cc23)c2oc3ccccc3c2c1]

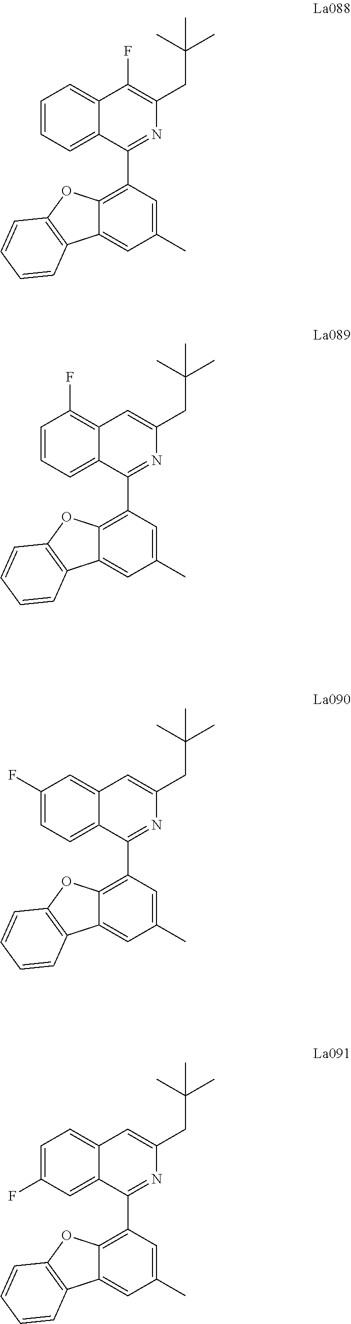 [SMILES: Cc1cc(-c2nc(CC(C)(C)C)c(F)c3ccccc23)c2oc3ccccc3c2c1 ; Cc1cc(-c2nc(CC(C)(C)C)cc3c(F)cccc23)c2oc3ccccc3c2c1 ; Cc1cc(-c2nc(CC(C)(C)C)cc3cc(F)ccc23)c2oc3ccccc3c2c1 ; Cc1cc(-c2nc(CC(C)(C)C)cc3ccc(F)cc23)c2oc3ccccc3c2c1]

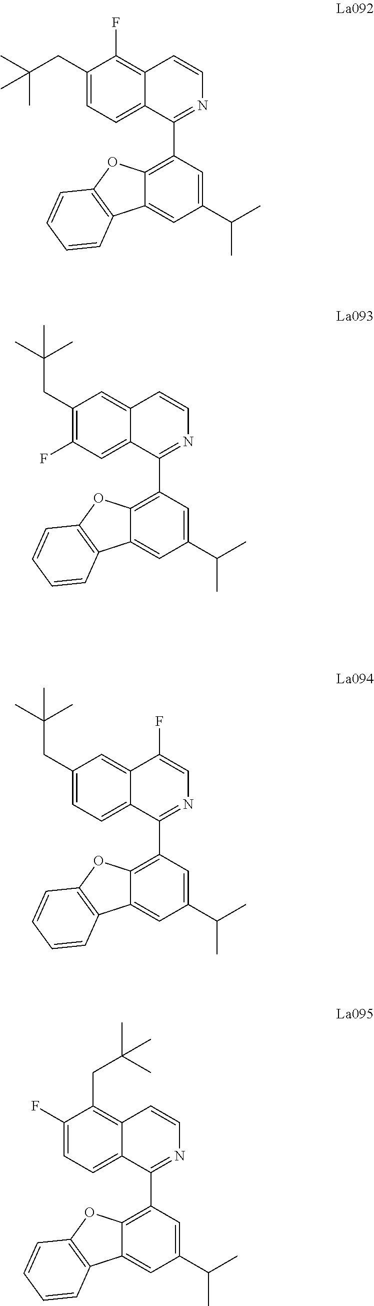

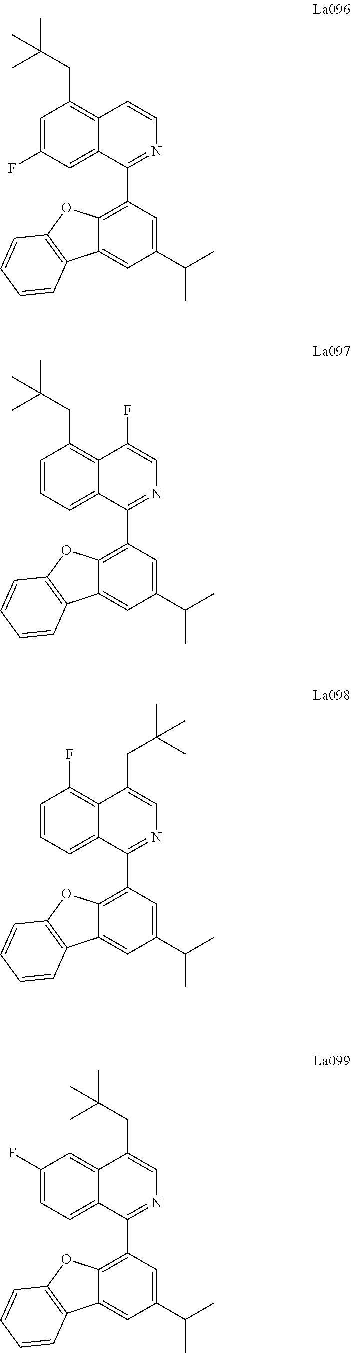 [SMILES: CC(C)c1cc(-c2nccc3c(CC(C)(C)C)cc(F)cc23)c2oc3ccccc3c2c1 ; CC(C)c1cc(-c2ncc(F)c3c(CC(C)(C)C)cccc23)c2oc3ccccc3c2c1 ; CC(C)c1cc(-c2ncc(CC(C)(C)C)c3c(F)cccc23)c2oc3ccccc3c2c1 ; CC(C)c1cc(-c2ncc(CC(C)(C)C)c3cc(F)ccc23)c2oc3ccccc3c2c1]

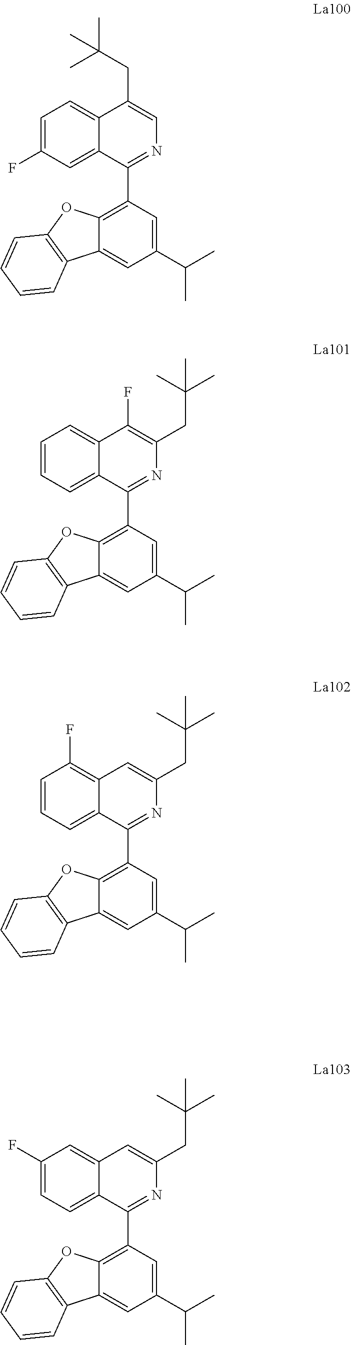 [SMILES: CC(C)c1cc(-c2ncc(CC(C)(C)C)c3ccc(F)cc23)c2oc3ccccc3c2c1 ; CC(C)c1cc(-c2nc(CC(C)(C)C)c(F)c3ccccc23)c2oc3ccccc3c2c1 ; CC(C)c1cc(-c2nc(CC(C)(C)C)cc3c(F)cccc23)c2oc3ccccc3c2c1 ; CC(C)c1cc(-c2nc(CC(C)(C)C)cc3cc(F)ccc23)c2oc3ccccc3c2c1]

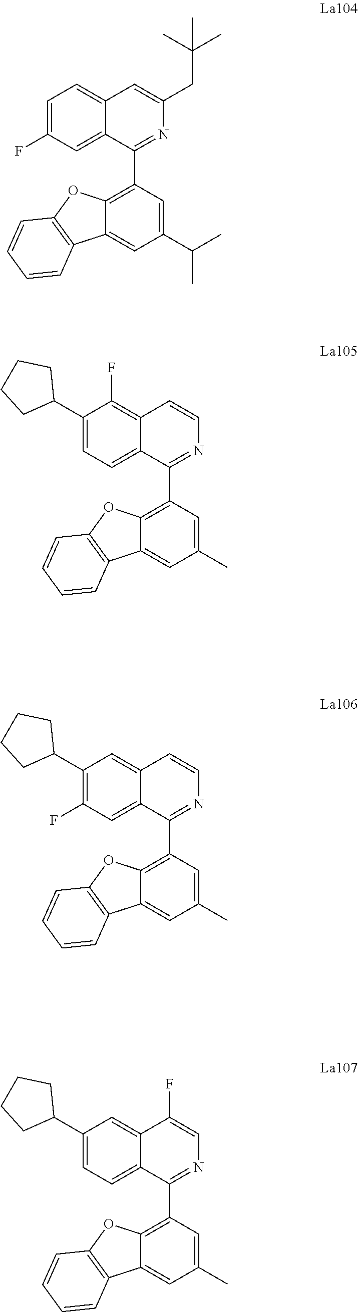 [SMILES: CC(C)c1cc(-c2nc(CC(C)(C)C)cc3ccc(F)cc23)c2oc3ccccc3c2c1 ; Cc1cc(-c2nccc3c(F)c(C4CCCC4)ccc23)c2oc3ccccc3c2c1 ; Cc1cc(-c2nccc3cc(C4CCCC4)c(F)cc23)c2oc3ccccc3c2c1 ; Cc1cc(-c2ncc(F)c3cc(C4CCCC4)ccc23)c2oc3ccccc3c2c1]

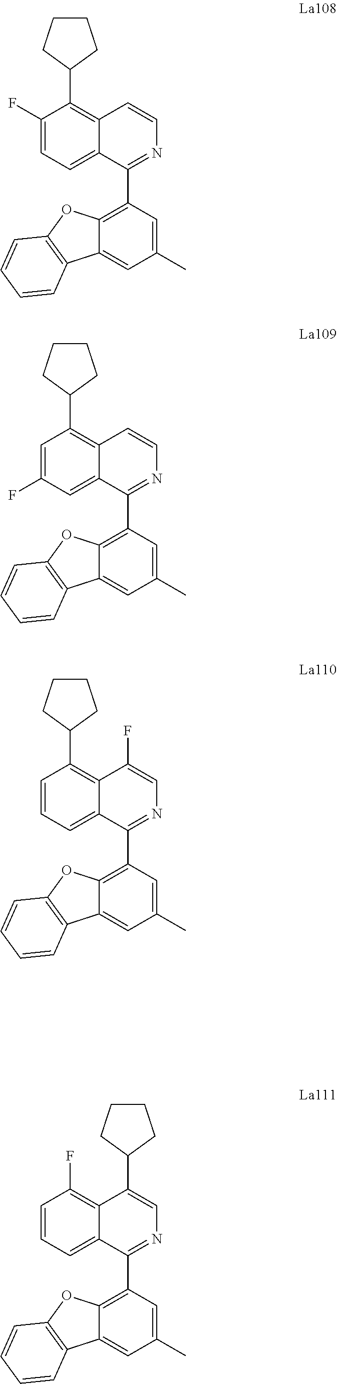 [SMILES: Cc1cc(-c2nccc3c(C4CCCC4)c(F)ccc23)c2oc3ccccc3c2c1 ; Cc1cc(-c2nccc3c(C4CCCC4)cc(F)cc23)c2oc3ccccc3c2c1 ; Cc1cc(-c2ncc(F)c3c(C4CCCC4)cccc23)c2oc3ccccc3c2c1 ; Cc1cc(-c2ncc(C3CCCC3)c3c(F)cccc23)c2oc3ccccc3c2c1]

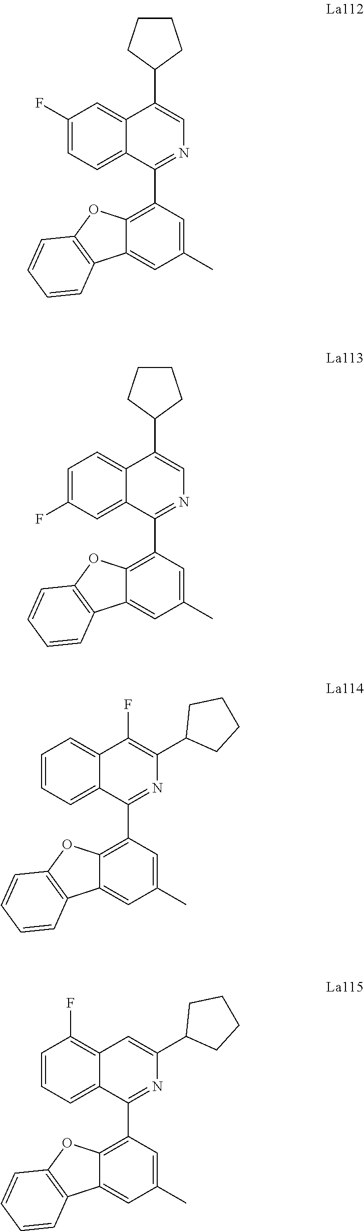 [SMILES: Cc1cc(-c2ncc(C3CCCC3)c3cc(F)ccc23)c2oc3ccccc3c2c1 ; Cc1cc(-c2ncc(C3CCCC3)c3ccc(F)cc23)c2oc3ccccc3c2c1 ; Cc1cc(-c2nc(C3CCCC3)c(F)c3ccccc23)c2oc3ccccc3c2c1 ; Cc1cc(-c2nc(C3CCCC3)cc3c(F)cccc23)c2oc3ccccc3c2c1]

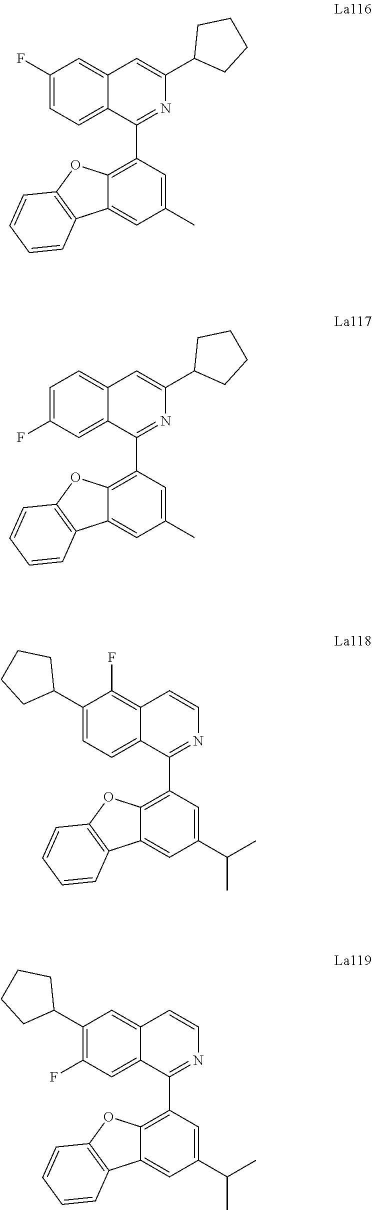 [SMILES: Cc1cc(-c2nc(C3CCCC3)cc3cc(F)ccc23)c2oc3ccccc3c2c1 ; Cc1cc(-c2nc(C3CCCC3)cc3ccc(F)cc23)c2oc3ccccc3c2c1 ; CC(C)c1cc(-c2nccc3c(F)c(C4CCCC4)ccc23)c2oc3ccccc3c2c1 ; CC(C)c1cc(-c2nccc3cc(C4CCCC4)c(F)cc23)c2oc3ccccc3c2c1]

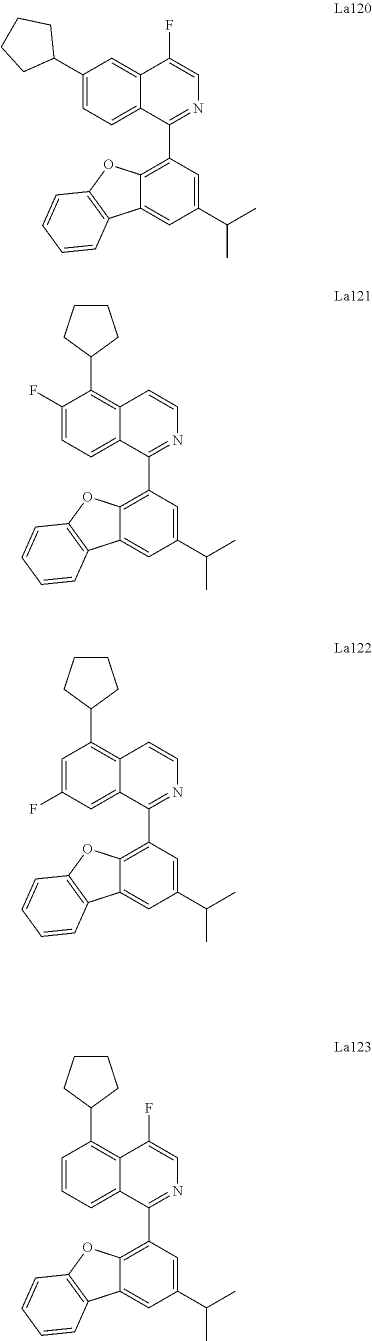 [SMILES: CC(C)c1cc(-c2ncc(F)c3cc(C4CCCC4)ccc23)c2oc3ccccc3c2c1 ; CC(C)c1cc(-c2nccc3c(C4CCCC4)c(F)ccc23)c2oc3ccccc3c2c1 ; CC(C)c1cc(-c2nccc3c(C4CCCC4)cc(F)cc23)c2oc3ccccc3c2c1 ; CC(C)c1cc(-c2ncc(F)c3c(C4CCCC4)cccc23)c2oc3ccccc3c2c1]

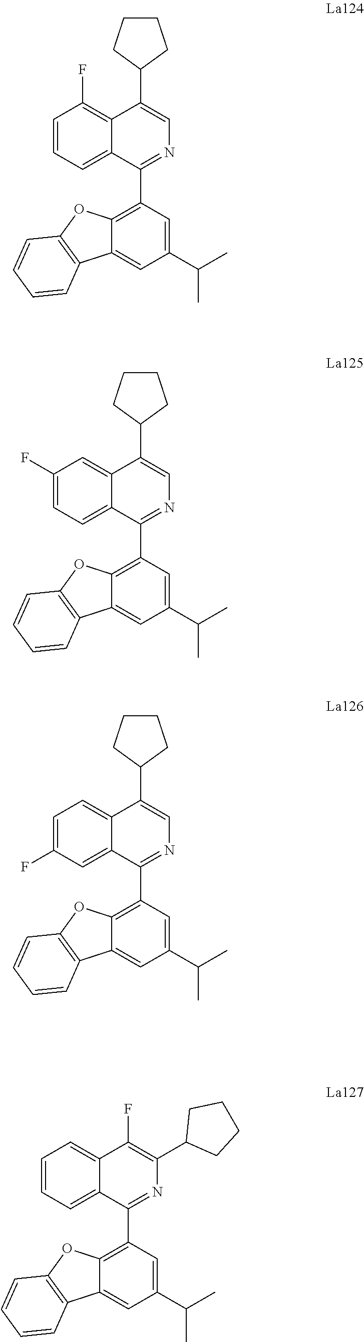 [SMILES: CC(C)c1cc(-c2ncc(C3CCCC3)c3c(F)cccc23)c2oc3ccccc3c2c1 ; CC(C)c1cc(-c2ncc(C3CCCC3)c3cc(F)ccc23)c2oc3ccccc3c2c1 ; CC(C)c1cc(-c2ncc(C3CCCC3)c3ccc(F)cc23)c2oc3ccccc3c2c1 ; CC(C)c1cc(-c2nc(C3CCCC3)c(F)c3ccccc23)c2oc3ccccc3c2c1]

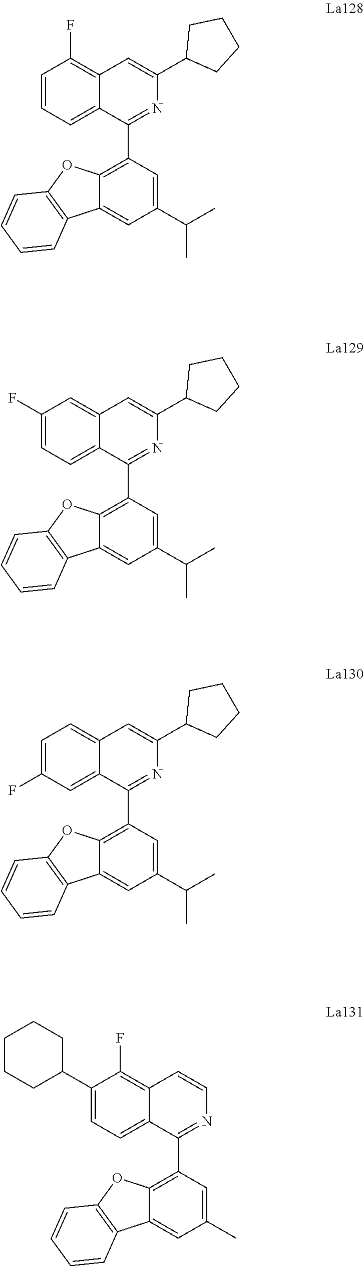 [SMILES: CC(C)c1cc(-c2nc(C3CCCC3)cc3c(F)cccc23)c2oc3ccccc3c2c1 ; CC(C)c1cc(-c2nc(C3CCCC3)cc3cc(F)ccc23)c2oc3ccccc3c2c1 ; CC(C)c1cc(-c2nc(C3CCCC3)cc3ccc(F)cc23)c2oc3ccccc3c2c1 ; Cc1cc(-c2nccc3c(F)c(C4CCCCC4)ccc23)c2oc3ccccc3c2c1]

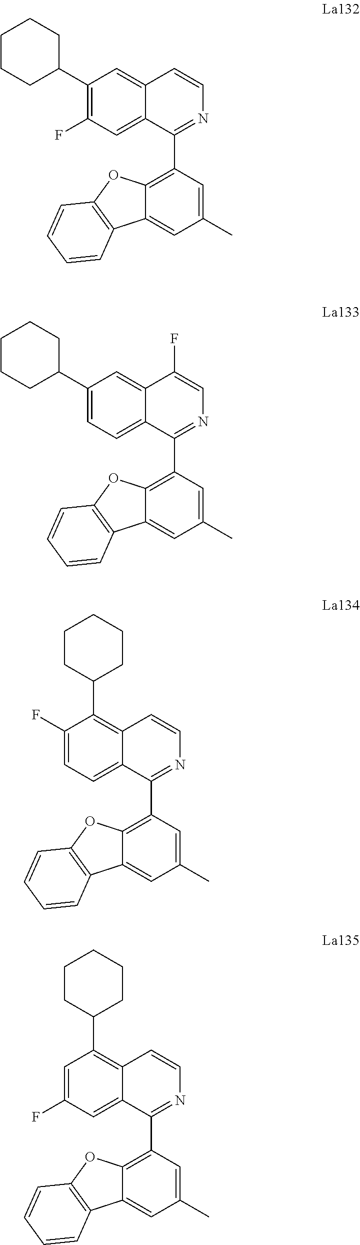 [SMILES: Cc1cc(-c2nccc3cc(C4CCCCC4)c(F)cc23)c2oc3ccccc3c2c1 ; Cc1cc(-c2ncc(F)c3cc(C4CCCCC4)ccc23)c2oc3ccccc3c2c1 ; Cc1cc(-c2nccc3c(C4CCCCC4)c(F)ccc23)c2oc3ccccc3c2c1 ; Cc1cc(-c2nccc3c(C4CCCCC4)cc(F)cc23)c2oc3ccccc3c2c1]

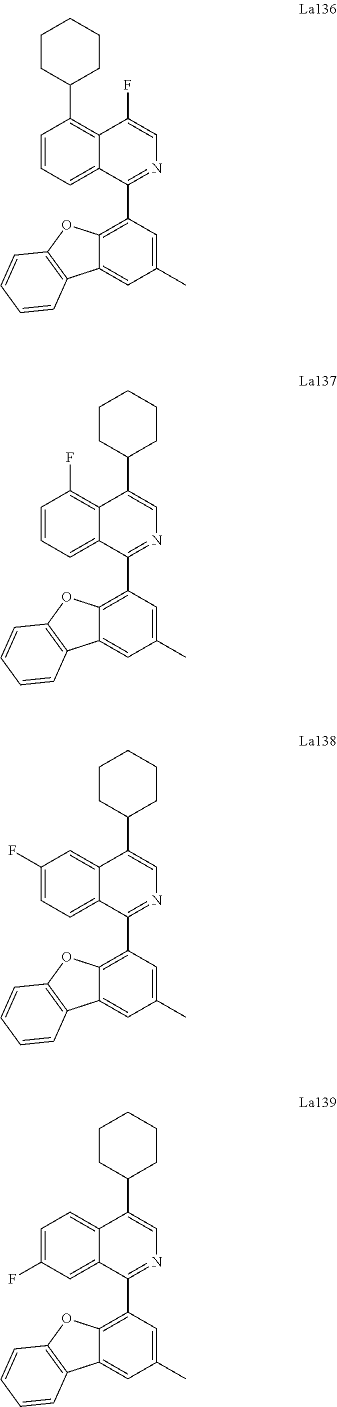 [SMILES: Cc1cc(-c2ncc(F)c3c(C4CCCCC4)cccc23)c2oc3ccccc3c2c1 ; Cc1cc(-c2ncc(C3CCCCC3)c3c(F)cccc23)c2oc3ccccc3c2c1 ; Cc1cc(-c2ncc(C3CCCCC3)c3cc(F)ccc23)c2oc3ccccc3c2c1 ; Cc1cc(-c2ncc(C3CCCCC3)c3ccc(F)cc23)c2oc3ccccc3c2c1]

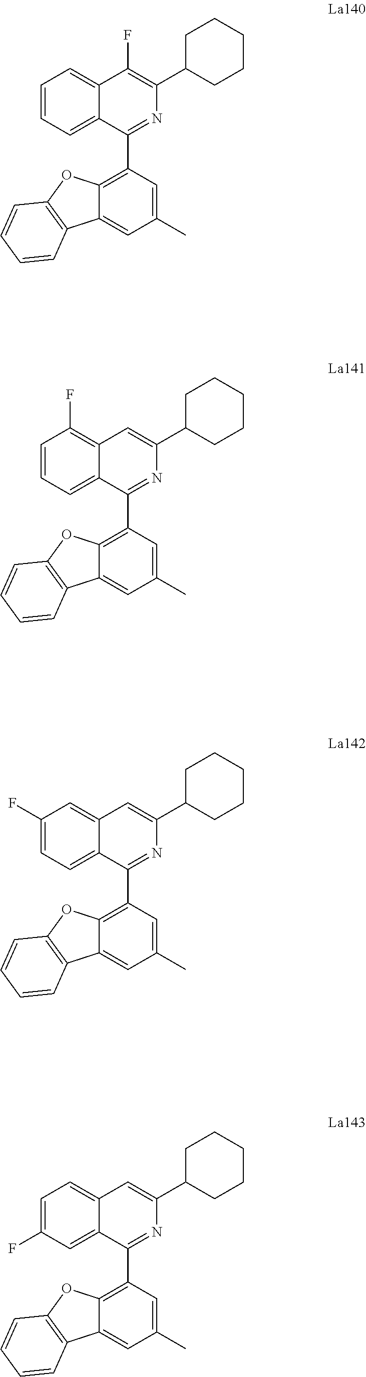 [SMILES: Cc1cc(-c2nc(C3CCCCC3)c(F)c3ccccc23)c2oc3ccccc3c2c1 ; Cc1cc(-c2nc(C3CCCCC3)cc3c(F)cccc23)c2oc3ccccc3c2c1 ; Cc1cc(-c2nc(C3CCCCC3)cc3cc(F)ccc23)c2oc3ccccc3c2c1 ; Cc1cc(-c2nc(C3CCCCC3)cc3ccc(F)cc23)c2oc3ccccc3c2c1]

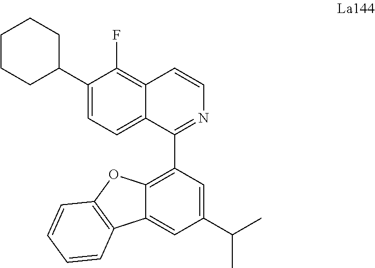 [SMILES: CC(C)c1cc(-c2nccc3c(F)c(C4CCCCC4)ccc23)c2oc3ccccc3c2c1]

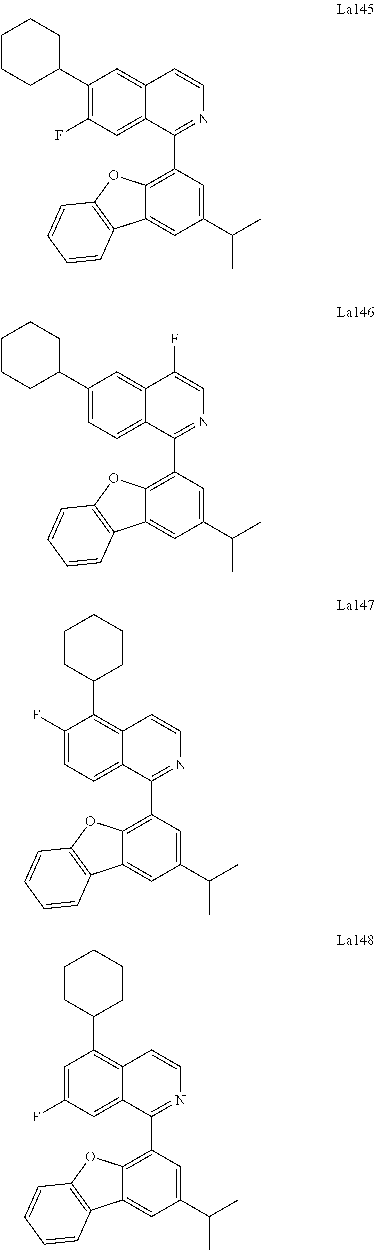 [SMILES: CC(C)c1cc(-c2nccc3cc(C4CCCCC4)c(F)cc23)c2oc3ccccc3c2c1 ; CC(C)c1cc(-c2ncc(F)c3cc(C4CCCCC4)ccc23)c2oc3ccccc3c2c1 ; CC(C)c1cc(-c2nccc3c(C4CCCCC4)c(F)ccc23)c2oc3ccccc3c2c1 ; CC(C)c1cc(-c2nccc3c(C4CCCCC4)cc(F)cc23)c2oc3ccccc3c2c1]

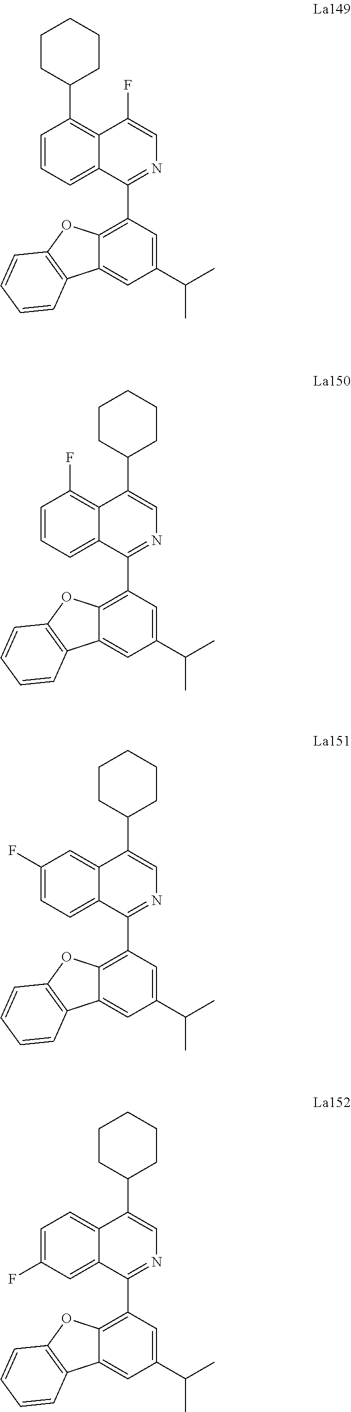 [SMILES: CC(C)c1cc(-c2ncc(F)c3c(C4CCCCC4)cccc23)c2oc3ccccc3c2c1 ; CC(C)c1cc(-c2ncc(C3CCCCC3)c3c(F)cccc23)c2oc3ccccc3c2c1 ; CC(C)c1cc(-c2ncc(C3CCCCC3)c3cc(F)ccc23)c2oc3ccccc3c2c1 ; CC(C)c1cc(-c2ncc(C3CCCCC3)c3ccc(F)cc23)c2oc3ccccc3c2c1]

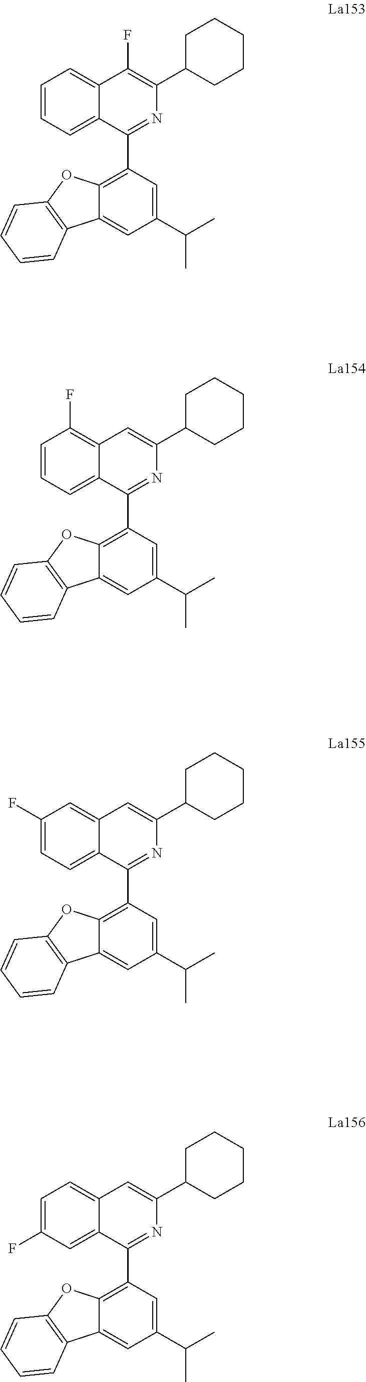 [SMILES: CC(C)c1cc(-c2nc(C3CCCCC3)c(F)c3ccccc23)c2oc3ccccc3c2c1 ; CC(C)c1cc(-c2nc(C3CCCCC3)cc3c(F)cccc23)c2oc3ccccc3c2c1 ; CC(C)c1cc(-c2nc(C3CCCCC3)cc3cc(F)ccc23)c2oc3ccccc3c2c1 ; CC(C)c1cc(-c2nc(C3CCCCC3)cc3ccc(F)cc23)c2oc3ccccc3c2c1]

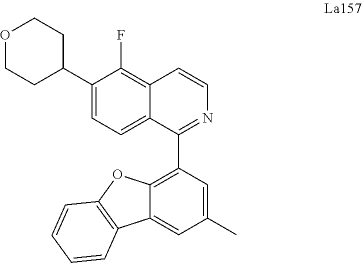 [SMILES: Cc1cc(-c2nccc3c(F)c(C4CCOCC4)ccc23)c2oc3ccccc3c2c1]

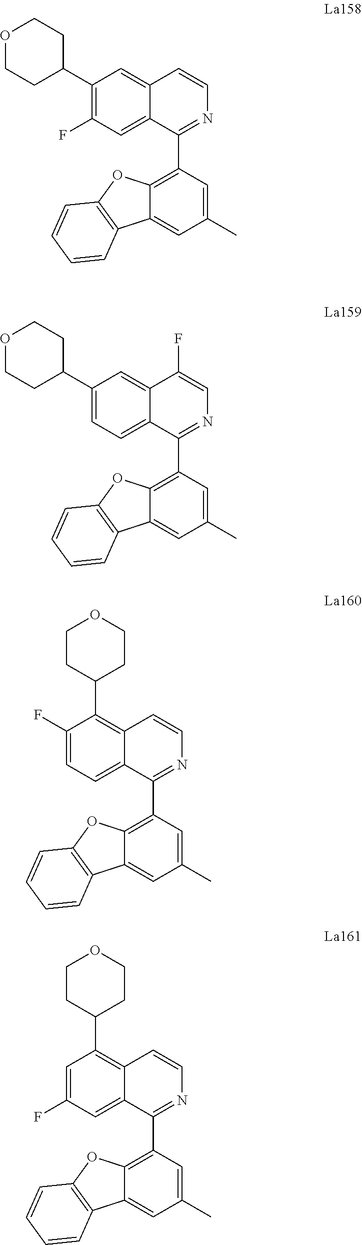 [SMILES: Cc1cc(-c2nccc3cc(C4CCOCC4)c(F)cc23)c2oc3ccccc3c2c1 ; Cc1cc(-c2ncc(F)c3cc(C4CCOCC4)ccc23)c2oc3ccccc3c2c1 ; Cc1cc(-c2nccc3c(C4CCOCC4)c(F)ccc23)c2oc3ccccc3c2c1 ; Cc1cc(-c2nccc3c(C4CCOCC4)cc(F)cc23)c2oc3ccccc3c2c1]

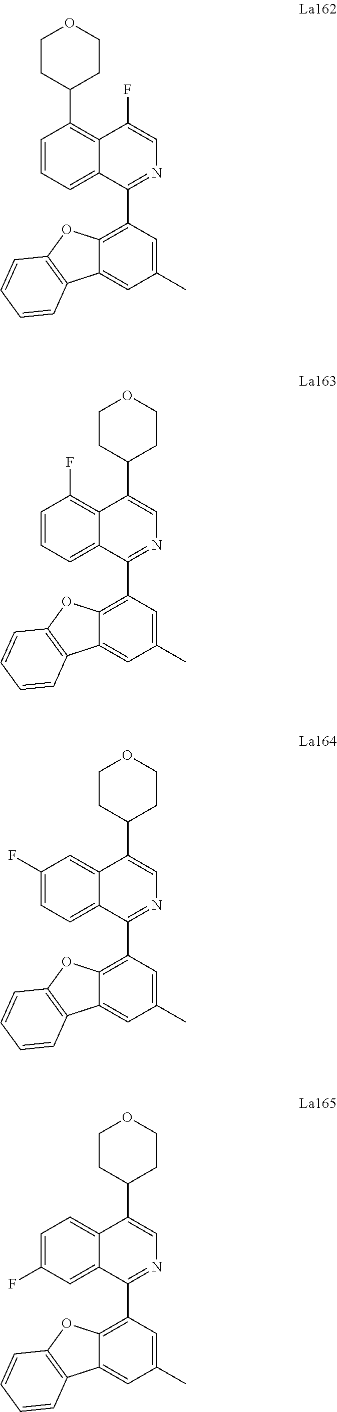 [SMILES: Cc1cc(-c2ncc(F)c3c(C4CCOCC4)cccc23)c2oc3ccccc3c2c1 ; Cc1cc(-c2ncc(C3CCOCC3)c3c(F)cccc23)c2oc3ccccc3c2c1 ; Cc1cc(-c2ncc(C3CCOCC3)c3cc(F)ccc23)c2oc3ccccc3c2c1 ; Cc1cc(-c2ncc(C3CCOCC3)c3ccc(F)cc23)c2oc3ccccc3c2c1]

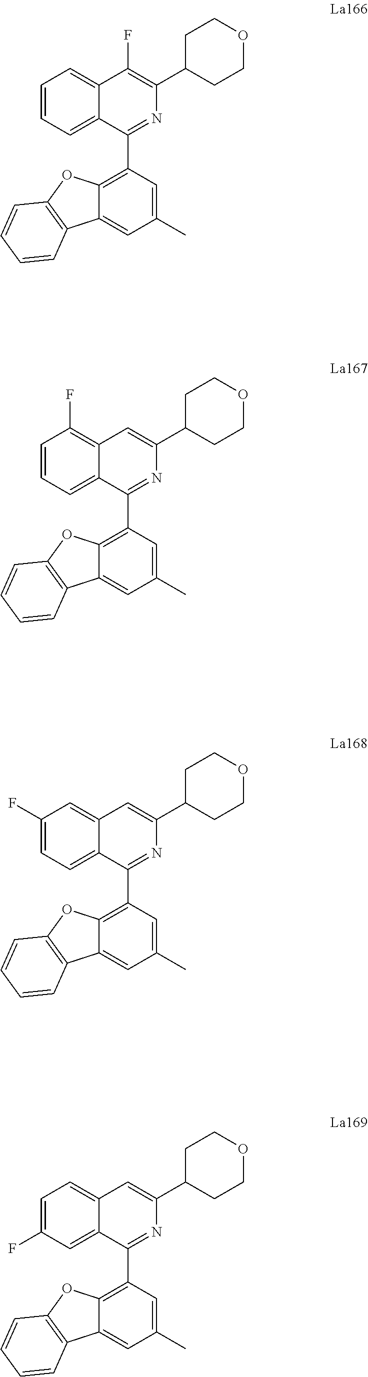 [SMILES: Cc1cc(-c2nc(C3CCOCC3)c(F)c3ccccc23)c2oc3ccccc3c2c1 ; Cc1cc(-c2nc(C3CCOCC3)cc3c(F)cccc23)c2oc3ccccc3c2c1 ; Cc1cc(-c2nc(C3CCOCC3)cc3cc(F)ccc23)c2oc3ccccc3c2c1 ; Cc1cc(-c2nc(C3CCOCC3)cc3ccc(F)cc23)c2oc3ccccc3c2c1]

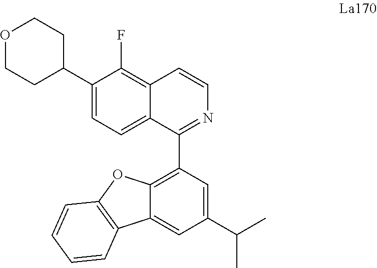 [SMILES: CC(C)c1cc(-c2nccc3c(F)c(C4CCOCC4)ccc23)c2oc3ccccc3c2c1]

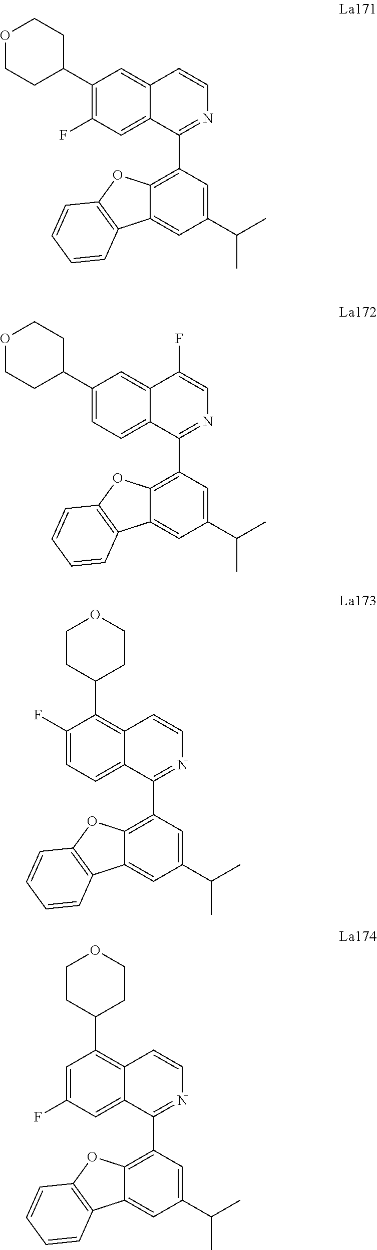 [SMILES: CC(C)c1cc(-c2nccc3cc(C4CCOCC4)c(F)cc23)c2oc3ccccc3c2c1 ; CC(C)c1cc(-c2ncc(F)c3cc(C4CCOCC4)ccc23)c2oc3ccccc3c2c1 ; CC(C)c1cc(-c2nccc3c(C4CCOCC4)c(F)ccc23)c2oc3ccccc3c2c1 ; CC(C)c1cc(-c2nccc3c(C4CCOCC4)cc(F)cc23)c2oc3ccccc3c2c1]

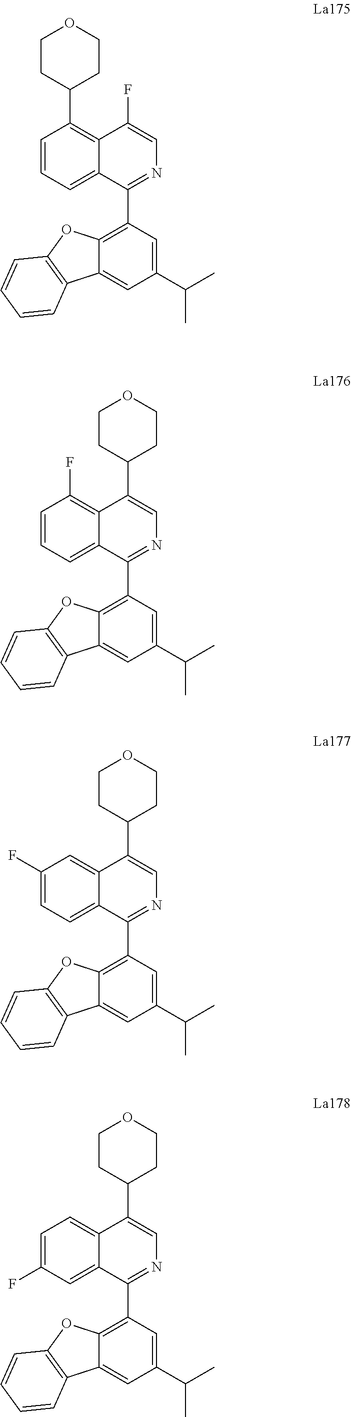 [SMILES: CC(C)c1cc(-c2ncc(F)c3c(C4CCOCC4)cccc23)c2oc3ccccc3c2c1 ; CC(C)c1cc(-c2ncc(C3CCOCC3)c3c(F)cccc23)c2oc3ccccc3c2c1 ; CC(C)c1cc(-c2ncc(C3CCOCC3)c3cc(F)ccc23)c2oc3ccccc3c2c1 ; CC(C)c1cc(-c2ncc(C3CCOCC3)c3ccc(F)cc23)c2oc3ccccc3c2c1]

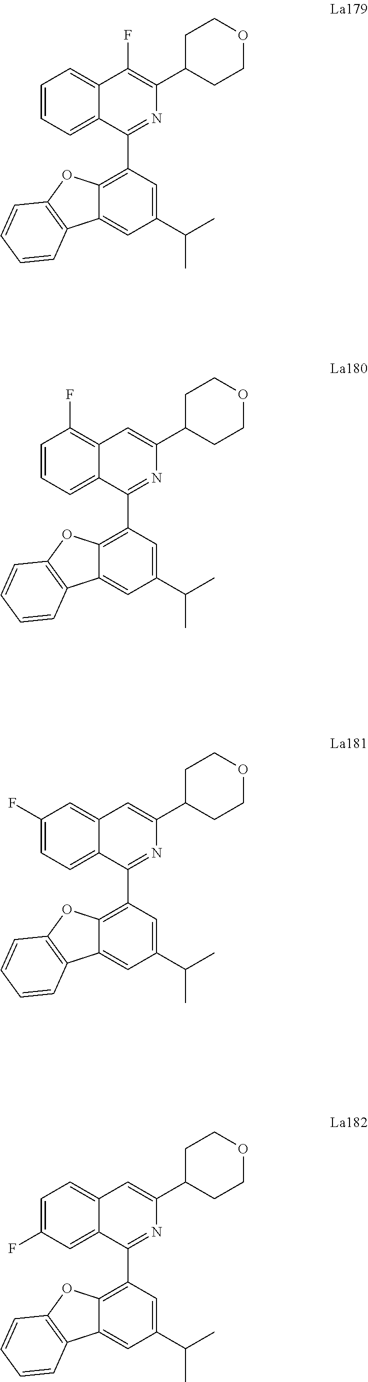 [SMILES: CC(C)c1cc(-c2nc(C3CCOCC3)c(F)c3ccccc23)c2oc3ccccc3c2c1 ; CC(C)c1cc(-c2nc(C3CCOCC3)cc3c(F)cccc23)c2oc3ccccc3c2c1 ; CC(C)c1cc(-c2nc(C3CCOCC3)cc3cc(F)ccc23)c2oc3ccccc3c2c1 ; CC(C)c1cc(-c2nc(C3CCOCC3)cc3ccc(F)cc23)c2oc3ccccc3c2c1]

12. An electroluminescent device, comprising a cathode, an anode, and organic layers arranged between the cathode and the anode, wherein at least one of the organic layers comprises the metal complex according to claim 1.

13. The electroluminescent device according to claim 12, wherein the organic layers comprise a light-emitting layer, and the metal complex according to claim 1 is used a red light-emitting doping material for the light-emitting layer; or the organic layers comprise a hole injection layer, and the metal complex according to claim 1 is used as a hole injection material in the hole injection layer.

14. A ligand La, having the following structure: 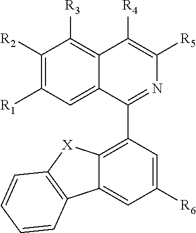
wherein R₁-R₆ and X are defined as described according to claim 1.

15. The metal complex according to claim 2, wherein the R₆ is substituted or unsubstituted alkyl containing no more than 4 carbon atoms on a main chain, or substituted or unsubstituted cycloalkyl containing no more than 6 ring forming carbon atoms.

16. The metal complex according to claim 3, wherein the R₆ is substituted or unsubstituted alkyl containing no more than 4 carbon atoms on a main chain, or substituted or unsubstituted cycloalkyl containing no more than 6 ring forming carbon atoms.

17. The metal complex according to claim 4, wherein the R₆ is substituted or unsubstituted alkyl containing no more than 4 carbon atoms on a main chain, or substituted or unsubstituted cycloalkyl containing no more than 6 ring forming carbon atoms.

18. An electroluminescent device, comprising a cathode, an anode, and organic layers arranged between the cathode and the anode, wherein at least one of the organic layers comprises the metal complex according to claim 2.

19. An electroluminescent device, comprising a cathode, an anode, and organic layers arranged between the cathode and the anode, wherein at least one of the organic layers comprises the metal complex according to claim 3.

20. An electroluminescent device, comprising a cathode, an anode, and organic layers arranged between the cathode and the anode, wherein at least one of the organic layers comprises the metal complex according to claim 4.
